SEQUENCE LISTING (246 sequences)

SEQ ID NO: 1 - protein, 14 aa, Artificial Sequence
  Description of Artificial Sequence Synthetic peptide
MHGKTQATSGTIQS

SEQ ID NO: 2 - protein, 7 aa, Artificial Sequence
  Description of Artificial Sequence Synthetic peptide
QQSWPIS

SEQ ID NO: 3 - protein, 227 aa, Artificial Sequence
  Description of Artificial Sequence Synthetic polypeptide
QQSWPISGSGQQSWPISGSGQQSWPISGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGQQSWPISGSGQQSWPISGSGQQSWPIS

SEQ ID NO: 4 - protein, 5 aa, Artificial Sequence
  Description of Artificial Sequence Synthetic peptide
EAAAR

SEQ ID NO: 5 - protein, 30 aa, Artificial Sequence
  Description of Artificial Sequence Synthetic polypeptide
QQSWPISGSGQQSWPISGSGQQSWPISGSG

SEQ ID NO: 6 - DNA, 19 bp, Artificial Sequence
  Description of Artificial Sequence Synthetic oligonucleotide
acgtagcaggtgacaggtt

SEQ ID NO: 7 - DNA, 14 bp, Artificial Sequence
  Description of Artificial Sequence Synthetic oligonucleotide
aacctgtcacctgc

SEQ ID NO: 8 - DNA, 45 bp, Artificial Sequence
  Description of Artificial Sequence Synthetic probe
cgatcaggccttcacagaggaagtatcctgtcgtttagcataccc

SEQ ID NO: 9 - DNA, 45 bp, Unknown
  Description of Unknown target sequence
gggtatgctaaacgacaggatacttcctctgtgaaggcctgatcg

SEQ ID NO: 10 - DNA, 20 bp, Unknown
  Description of Unknown target sequence
cctctgtgaaggcctgatcg

SEQ ID NO: 11 - DNA, 19 bp, Unknown
  Description of Unknown target sequence
cctctgtgaaggcctgatc

SEQ ID NO: 12 - DNA, 18 bp, Unknown
  Description of Unknown target sequence
cctctgtgaaggcctgat

SEQ ID NO: 13 - DNA, 17 bp, Unknown
  Description of Unknown target sequence
cctctgtgaaggcctga

SEQ ID NO: 14 - DNA, 16 bp, Unknown
  Description of Unknown target sequence
cctctgtgaaggcctg

SEQ ID NO: 15 - DNA, 15 bp, Unknown
  Description of Unknown target sequence
cctctgtgaaggcct

SEQ ID NO: 16 - DNA, 20 bp, Unknown
  Description of Unknown target sequence
tacttcctctgtgaaggcct

SEQ ID NO: 17 - DNA, 20 bp, Unknown
  Description of Unknown target sequence
caggatacttcctctgtgaa

SEQ ID NO: 18 - DNA, 20 bp, Unknown
  Description of Unknown target sequence
aacgacaggatacttcctct

SEQ ID NO: 19 - DNA, 20 bp, Unknown
  Description of Unknown target sequence
tgctaaacgacaggatactt

SEQ ID NO: 20 - DNA, 20 bp, Unknown
  Description of Unknown target sequence
gggtatgctaaacgacagga

SEQ ID NO: 21 - DNA, 20 bp, Unknown
  Description of Unknown target sequence
cctctgtgaaggcctgatcg

SEQ ID NO: 22 - DNA, 20 bp, Unknown
  Description of Unknown target sequence
cctcagagtacggcagatcg

SEQ ID NO: 23 - DNA, 20 bp, Unknown
  Description of Unknown target sequence
cctctctcatgccgtcatcg

SEQ ID NO: 24 - DNA, 20 bp, Unknown
  Description of Unknown target sequence
cctcagagaaggcctgatcg

SEQ ID NO: 25 - DNA, 20 bp, Unknown
  Description of Unknown target sequence
cctctgtgtacgcctgatcg

SEQ ID NO: 26 - DNA, 20 bp, Unknown
  Description of Unknown target sequence
cctctgtgaagggcagatcg

SEQ ID NO: 27 - DNA, 20 bp, Unknown
  Description of Unknown target sequence
cctcagagaagggcagatcg

SEQ ID NO: 28 - DNA, 20 bp, Unknown
  Description of Unknown target sequence
cctctgtgatgccgtcatcg

SEQ ID NO: 29 - DNA, 20 bp, Unknown
  Description of Unknown target sequence
cctcagagtacgcctgatcg

SEQ ID NO: 30 - DNA, 20 bp, Unknown
  Description of Unknown target sequence
ccagtgtgaaggcctgtacg

SEQ ID NO: 31 - DNA, 20 bp, Unknown
  Description of Unknown target sequence
ccagagtgaaggcctgatcg

SEQ ID NO: 32 - DNA, 20 bp, Unknown
  Description of Unknown target sequence
cctctcacaaggcctgatcg

SEQ ID NO: 33 - DNA, 20 bp, Unknown
  Description of Unknown target sequence
cctctgtgttcgcctgatcg

SEQ ID NO: 34 - DNA, 20 bp, Unknown
  Description of Unknown target sequence
cctctgtgaagcggtgatcg

SEQ ID NO: 35 - DNA, 20 bp, Unknown
  Description of Unknown target sequence
cctctgtgaaggccacttcg

SEQ ID NO: 36 - DNA, 20 bp, Unknown
  Description of Unknown target sequence
cctctgtgaacccctgatcg

SEQ ID NO: 37 - DNA, 20 bp, Unknown
  Description of Unknown target sequence
cctctgtgaaggggtgatcg

SEQ ID NO: 38 - DNA, 20 bp, Unknown
  Description of Unknown target sequence
cctctgtgaaggcctactcg

SEQ ID NO: 39 - DNA, 20 bp, Unknown
  Description of Unknown target sequence
cctgacagaaggcctgatcg

SEQ ID NO: 40 - DNA, 20 bp, Unknown
  Description of Unknown target sequence
cctctgtgaaggccactacg

SEQ ID NO: 41 - DNA, 20 bp, Unknown
  Description of Unknown target sequence
ccactgtgaaggcctgatcg

SEQ ID NO: 42 - DNA, 20 bp, Unknown
  Description of Unknown target sequence
cctgtgtgaaggcctgatcg

SEQ ID NO: 43 - DNA, 20 bp, Unknown
  Description of Unknown target sequence
cctcagtgaaggcctgatcg

SEQ ID NO: 44 - DNA, 20 bp, Unknown
  Description of Unknown target sequence
cctctctgaaggcctgatcg

SEQ ID NO: 45 - DNA, 20 bp, Unknown
  Description of Unknown target sequence
cctctgagaaggcctgatcg

SEQ ID NO: 46 - DNA, 20 bp, Unknown
  Description of Unknown target sequence
cctctgtcaaggcctgatcg

SEQ ID NO: 47 - DNA, 20 bp, Unknown
  Description of Unknown target sequence
cctctgtgtaggcctgatcg

SEQ ID NO: 48 - DNA, 20 bp, Unknown
  Description of Unknown target sequence
cctctgtgatggcctgatcg

SEQ ID NO: 49 - DNA, 20 bp, Unknown
  Description of Unknown target sequence
cctctgtgaacgcctgatcg

SEQ ID NO: 50 - DNA, 20 bp, Unknown
  Description of Unknown target sequence
cctctgtgaagccctgatcg

SEQ ID NO: 51 - DNA, 20 bp, Unknown
  Description of Unknown target sequence
cctctgtgaagggctgatcg

SEQ ID NO: 52 - DNA, 20 bp, Unknown
  Description of Unknown target sequence
cctctgtgaaggcgtgatcg

SEQ ID NO: 53 - DNA, 20 bp, Unknown
  Description of Unknown target sequence
cctctgtgaaggccagatcg

SEQ ID NO: 54 - DNA, 20 bp, Unknown
  Description of Unknown target sequence
cctctgtgaaggcctcatcg

SEQ ID NO: 55 - DNA, 20 bp, Unknown
  Description of Unknown target sequence
cctctgtgaaggcctgttcg

SEQ ID NO: 56 - DNA, 20 bp, Unknown
  Description of Unknown target sequence
cctctgtgaaggcctgaacg

SEQ ID NO: 57 - DNA, 13 bp, Artificial Sequence
  Description of Artificial Sequence Synthetic probe
ataggacaccgtt

SEQ ID NO: 58 - DNA, 13 bp, Artificial Sequence
  Description of Artificial Sequence Synthetic oligonucleotide
tatcctgtggcaa

SEQ ID NO: 59 - DNA, 13 bp, Artificial Sequence
  Description of Artificial Sequence Synthetic oligonucleotide
ataggacaccgtt

SEQ ID NO: 60 - DNA, 25 bp, Artificial Sequence
  Description of Artificial Sequence Synthetic primer
caagaccaatcytgtcacctctgac

SEQ ID NO: 61 - DNA, 23 bp, Artificial Sequence
  Description of Artificial Sequence Synthetic primer
gcattytggacaaavcgtctacg

SEQ ID NO: 62 - DNA, 24 bp, Artificial Sequence
  Description of Artificial Sequence Synthetic probe
tgcagtcctcgctcactgggcacg

SEQ ID NO: 63 - DNA, 25 bp, Artificial Sequence
  Description of Artificial Sequence Synthetic primer
caagaccaatyctgtcacctytgac

SEQ ID NO: 64 - DNA, 23 bp, Artificial Sequence
  Description of Artificial Sequence Synthetic primer
gcattttggataaagcgtctacg

SEQ ID NO: 65 - DNA, 24 bp, Artificial Sequence
  Description of Artificial Sequence Synthetic probe
tgcagtcctcgctcactgggcacg

SEQ ID NO: 66 - DNA, 22 bp, Artificial Sequence
  Description of Artificial Sequence Synthetic primer
tcctcaaytcactcttcgagcg

SEQ ID NO: 67 - DNA, 21 bp, Artificial Sequence
  Description of Artificial Sequence Synthetic primer
cggtgctcttgaccaaattgg

SEQ ID NO: 68 - DNA, 27 bp, Artificial Sequence
  Description of Artificial Sequence Synthetic probe
ccaattcgagcagctgaaactgcggtg

SEQ ID NO: 69 - DNA, 19 bp, Artificial Sequence
  Description of Artificial Sequence Synthetic primer
agatttggacctgcgagcg

SEQ ID NO: 70 - DNA, 20 bp, Artificial Sequence
  Description of Artificial Sequence Synthetic primer
gagcggctgtctccacaagt

SEQ ID NO: 71 - DNA, 23 bp, Artificial Sequence
  Description of Artificial Sequence Synthetic probe
ttctgacctgaaggctctgcgcg

SEQ ID NO: 72 - DNA, 20 bp, Artificial Sequence
  Description of Artificial Sequence Synthetic primer
gaccccaaaatcagcgaaat

SEQ ID NO: 73 - DNA, 24 bp, Artificial Sequence
  Description of Artificial Sequence Synthetic primer
tctggttactgccagttgaatctg

SEQ ID NO: 74 - DNA, 24 bp, Artificial Sequence
  Description of Artificial Sequence Synthetic probe
accccgcattacgtttggtggacc

SEQ ID NO: 75 - DNA, 20 bp, Artificial Sequence
  Description of Artificial Sequence Synthetic primer
ttacaaacattggccgcaaa

SEQ ID NO: 76 - DNA, 18 bp, Artificial Sequence
  Description of Artificial Sequence Synthetic primer
gcgcgacattccgaagaa

SEQ ID NO: 77 - DNA, 23 bp, Artificial Sequence
  Description of Artificial Sequence Synthetic probe
acaatttgcccccagcgcttcag

SEQ ID NO: 78 - DNA, 19 bp, Artificial Sequence
  Description of Artificial Sequence Synthetic primer
ggtaactggtatgatttcg

SEQ ID NO: 79 - DNA, 20 bp, Artificial Sequence
  Description of Artificial Sequence Synthetic primer
ctggtcaaggttaatatagg

SEQ ID NO: 80 - DNA, 19 bp, Artificial Sequence
  Description of Artificial Sequence Synthetic probe
tcatacaaaccacgccagg

SEQ ID NO: 81 - DNA, 26 bp, Artificial Sequence
  Description of Artificial Sequence Synthetic primer
acaggtacgttaatagttaatagcgt

SEQ ID NO: 82 - DNA, 22 bp, Artificial Sequence
  Description of Artificial Sequence Synthetic primer
atattgcagcagtacgcacaca

SEQ ID NO: 83 - DNA, 26 bp, Artificial Sequence
  Description of Artificial Sequence Synthetic probe
acactagccatccttactgcgcttcg

SEQ ID NO: 84 - DNA, 25 bp, Artificial Sequence
  Description of Artificial Sequence Synthetic primer
acaatctaaaacaacaactctatgc

SEQ ID NO: 85 - DNA, 20 bp, Artificial Sequence
  Description of Artificial Sequence Synthetic primer
gtgtatttgctggatgacag

SEQ ID NO: 86 - DNA, 38 bp, Artificial Sequence
  Description of Artificial Sequence Synthetic probe
atgcataactatactccatagtccagatggagcctgaa

SEQ ID NO: 87 - DNA, 31 bp, Artificial Sequence
  Description of Artificial Sequence Synthetic probe
gcaccccgcattacgtttggtggaccctcag

SEQ ID NO: 88 - DNA, 24 bp, Artificial Sequence
  Description of Artificial Sequence Synthetic probe
accccgcattacgtttggtggacc

SEQ ID NO: 89 - DNA, 21 bp, Artificial Sequence
  Description of Artificial Sequence Synthetic probe
ccgcattacgtttggtggacc

SEQ ID NO: 90 - DNA, 17 bp, Artificial Sequence
  Description of Artificial Sequence Synthetic probe
gcattacgtttggtgga

SEQ ID NO: 91 - DNA, 14 bp, Artificial Sequence
  Description of Artificial Sequence Synthetic probe
gcattacgtttggt

SEQ ID NO: 92 - DNA, 35 bp, Artificial Sequence
  Description of Artificial Sequence Synthetic primer
aaagacaagaccaatcttgtcacctctgactaagg

SEQ ID NO: 93 - DNA, 23 bp, Artificial Sequence
  Description of Artificial Sequence Synthetic primer
aaagagttgatttttgtggaaag

SEQ ID NO: 94 - DNA, 34 bp, Artificial Sequence
  Description of Artificial Sequence Synthetic primer
aaagatggccatcggatcctcaactcactcttcg

SEQ ID NO: 95 - DNA, 35 bp, Artificial Sequence
  Description of Artificial Sequence Synthetic primer
aaccattacagatgctgtagactgtgcacttgacc

SEQ ID NO: 96 - DNA, 18 bp, Artificial Sequence
  Description of Artificial Sequence Synthetic primer
aattgggagcttcgcagc

SEQ ID NO: 97 - DNA, 20 bp, Artificial Sequence
  Description of Artificial Sequence Synthetic primer
acaacaggatgggaacagtg

SEQ ID NO: 98 - DNA, 35 bp, Artificial Sequence
  Description of Artificial Sequence Synthetic primer
acaccgtttctatagattagctaatgagtgtgctc

SEQ ID NO: 99 - DNA, 26 bp, Artificial Sequence
  Description of Artificial Sequence Synthetic primer
acactagccatccttactgcgcttcg

SEQ ID NO: 100 - DNA, 25 bp, Artificial Sequence
  Description of Artificial Sequence Synthetic primer
acaggtacgttaatagttaatagcg

SEQ ID NO: 101 - DNA, 26 bp, Artificial Sequence
  Description of Artificial Sequence Synthetic primer
acaggtacgttaatagttaatagcgt

SEQ ID NO: 102 - DNA, 20 bp, Artificial Sequence
  Description of Artificial Sequence Synthetic primer
acgttgaaatccttcactgt

SEQ ID NO: 103 - DNA, 35 bp, Artificial Sequence
  Description of Artificial Sequence Synthetic primer
actcctggtgattcttcttcaggttggacagctgg

SEQ ID NO: 104 - DNA, 20 bp, Artificial Sequence
  Description of Artificial Sequence Synthetic primer
acttgtggagacagccgctc

SEQ ID NO: 105 - DNA, 23 bp, Artificial Sequence
  Description of Artificial Sequence Synthetic primer
acttgtggagacagccgctcacc

SEQ ID NO: 106 - DNA, 21 bp, Artificial Sequence
  Description of Artificial Sequence Synthetic primer
acttgttgctactgatgatct

SEQ ID NO: 107 - DNA, 35 bp, Artificial Sequence
  Description of Artificial Sequence Synthetic primer
acttgttgctactgatgatcttacagtggaggatg

SEQ ID NO: 108 - DNA, 35 bp, Artificial Sequence
  Description of Artificial Sequence Synthetic primer
agaaagtgaactcgtaatcggagctgtgatccttc

SEQ ID NO: 109 - DNA, 24 bp, Artificial Sequence
  Description of Artificial Sequence Synthetic primer
agaacttcacaactgctcctgcca

SEQ ID NO: 110 - DNA, 25 bp, Artificial Sequence
  Description of Artificial Sequence Synthetic primer
agaagttatttgactcctggtgatt

SEQ ID NO: 111 - DNA, 19 bp, Artificial Sequence
  Description of Artificial Sequence Synthetic primer
agaatggagaacgcagtgg

SEQ ID NO: 112 - DNA, 22 bp, Artificial Sequence
  Description of Artificial Sequence Synthetic primer
agagggagacaattagactggc

SEQ ID NO: 113 - DNA, 19 bp, Artificial Sequence
  Description of Artificial Sequence Synthetic primer
agatttggacctgcgagcg

SEQ ID NO: 114 - DNA, 20 bp, Artificial Sequence
  Description of Artificial Sequence Synthetic primer
agcagaaatggaacaagttg

SEQ ID NO: 115 - DNA, 24 bp, Artificial Sequence
  Description of Artificial Sequence Synthetic primer
aggtgtcactaagctattcaactg

SEQ ID NO: 116 - DNA, 25 bp, Artificial Sequence
  Description of Artificial Sequence Synthetic primer
aggtgtcactaagctattcaactgg

SEQ ID NO: 117 - DNA, 35 bp, Artificial Sequence
  Description of Artificial Sequence Synthetic primer
agtgctattggcaaaattcaagactcactttcttc

SEQ ID NO: 118 - DNA, 21 bp, Artificial Sequence
  Description of Artificial Sequence Synthetic primer
agtggaggatgaaaaagatgg

SEQ ID NO: 119 - DNA, 21 bp, Artificial Sequence
  Description of Artificial Sequence Synthetic primer
ataactacgatacgggagggc

SEQ ID NO: 120 - DNA, 23 bp, Artificial Sequence
  Description of Artificial Sequence Synthetic primer
atctgaattcttctagagttcct

SEQ ID NO: 121 - DNA, 23 bp, Artificial Sequence
  Description of Artificial Sequence Synthetic primer
atctttgactcaatttcctcact

SEQ ID NO: 122 - DNA, 23 bp, Artificial Sequence
  Description of Artificial Sequence Synthetic primer
atgaaccacaaatcattactaca

SEQ ID NO: 123 - DNA, 33 bp, Artificial Sequence
  Description of Artificial Sequence Synthetic primer
atggcggtgtttgcagatttggacctgcgagcg

SEQ ID NO: 124 - DNA, 35 bp, Artificial Sequence
  Description of Artificial Sequence Synthetic primer
atgtactcattcgtttcggaagagacaggtacgtt

SEQ ID NO: 125 - DNA, 35 bp, Artificial Sequence
  Description of Artificial Sequence Synthetic primer
atgtctgataatggaccccaaaatcagcgaaatgc

SEQ ID NO: 126 - DNA, 35 bp, Artificial Sequence
  Description of Artificial Sequence Synthetic primer
attggcaaaattcaagactcactttcttccacagc

SEQ ID NO: 127 - DNA, 19 bp, Artificial Sequence
  Description of Artificial Sequence Synthetic primer
attgggagcttcgcagcgt

SEQ ID NO: 128 - DNA, 35 bp, Artificial Sequence
  Description of Artificial Sequence Synthetic primer
attgtgtgcgtactgctgcaatattgttaacgtga

SEQ ID NO: 129 - DNA, 34 bp, Artificial Sequence
  Description of Artificial Sequence Synthetic primer
caaattgggagcttcgcagcgtgtagcaggtgac

SEQ ID NO: 130 - DNA, 19 bp, Artificial Sequence
  Description of Artificial Sequence Synthetic primer
caacgtgttgtagcttgtc

SEQ ID NO: 131 - DNA, 35 bp, Artificial Sequence
  Description of Artificial Sequence Synthetic primer
caacgtgttgtagcttgtcacaccgtttctataga

SEQ ID NO: 132 - DNA, 25 bp, Artificial Sequence
  Description of Artificial Sequence Synthetic primer
caagaccaatcttgtcacctctgac

SEQ ID NO: 133 - DNA, 35 bp, Artificial Sequence
  Description of Artificial Sequence Synthetic primer
caagctgtcacggccaatgttaatgcacttttatc

SEQ ID NO: 134 - DNA, 24 bp, Artificial Sequence
  Description of Artificial Sequence Synthetic primer
caaggacctgcctaaagaaatcac

SEQ ID NO: 135 - DNA, 35 bp, Artificial Sequence
  Description of Artificial Sequence Synthetic primer
caatttggtcaagagcaccgattatcaccagaaga

SEQ ID NO: 136 - DNA, 25 bp, Artificial Sequence
  Description of Artificial Sequence Synthetic primer
cacaaaggaatttttatgaaccaca

SEQ ID NO: 137 - DNA, 35 bp, Artificial Sequence
  Description of Artificial Sequence Synthetic primer
cacaaaggaatttttatgaaccacaaatcattact

SEQ ID NO: 138 - DNA, 23 bp, Artificial Sequence
  Description of Artificial Sequence Synthetic primer
cacctatctcagcgatctgtcta

SEQ ID NO: 139 - DNA, 23 bp, Artificial Sequence
  Description of Artificial Sequence Synthetic primer
cactggtttgtaacacaaaggaa

SEQ ID NO: 140 - DNA, 21 bp, Artificial Sequence
  Description of Artificial Sequence Synthetic primer
cagattcaactggcagtaacc

SEQ ID NO: 141 - DNA, 24 bp, Artificial Sequence
  Description of Artificial Sequence Synthetic primer
cagattcaactggcagtaaccaga

SEQ ID NO: 142 - DNA, 30 bp, Artificial Sequence
  Description of Artificial Sequence Synthetic primer
cagattcaactggcagtaaccagaatggag

SEQ ID NO: 143 - DNA, 35 bp, Artificial Sequence
  Description of Artificial Sequence Synthetic primer
cagattcaactggcagtaaccagaatggagaacgc

SEQ ID NO: 144 - DNA, 25 bp, Artificial Sequence
  Description of Artificial Sequence Synthetic primer
caggtggaacctcatcaggagatgc

SEQ ID NO: 145 - DNA, 21 bp, Artificial Sequence
  Description of Artificial Sequence Synthetic primer
catccatagttgcctgactcc

SEQ ID NO: 146 - DNA, 35 bp, Artificial Sequence
  Description of Artificial Sequence Synthetic primer
catggaatggctaaagacaagaccaatcttgtcac

SEQ ID NO: 147 - DNA, 35 bp, Artificial Sequence
  Description of Artificial Sequence Synthetic primer
cattacagatgctgtagactgtgcacttgaccctc

SEQ ID NO: 148 - DNA, 35 bp, Artificial Sequence
  Description of Artificial Sequence Synthetic primer
cattatgttaggacacgctagtgtacaagcagaaa

SEQ ID NO: 149 - DNA, 34 bp, Artificial Sequence
  Description of Artificial Sequence Synthetic primer
catttgtcaagctgtcacggccaatgttaatgca

SEQ ID NO: 150 - DNA, 22 bp, Artificial Sequence
  Description of Artificial Sequence Synthetic primer
ccaaggtttacccaataatact

SEQ ID NO: 151 - DNA, 35 bp, Artificial Sequence
  Description of Artificial Sequence Synthetic primer
ccaatgttaatgcacttttatctactgatggtaac

SEQ ID NO: 152 - DNA, 27 bp, Artificial Sequence
  Description of Artificial Sequence Synthetic primer
ccaattcgagcagctgaaactgcggtg

SEQ ID NO: 153 - DNA, 21 bp, Artificial Sequence
  Description of Artificial Sequence Synthetic primer
ccaatttggtcaagagcaccg

SEQ ID NO: 154 - DNA, 25 bp, Artificial Sequence
  Description of Artificial Sequence Synthetic primer
ccacaactgcttatgctaatagtgt

SEQ ID NO: 155 - DNA, 21 bp, Artificial Sequence
  Description of Artificial Sequence Synthetic primer
ccacggaagaactttatctct

SEQ ID NO: 156 - DNA, 28 bp, Artificial Sequence
  Description of Artificial Sequence Synthetic primer
ccatatattgaacaacccaaaagcatca

SEQ ID NO: 157 - DNA, 19 bp, Artificial Sequence
  Description of Artificial Sequence Synthetic primer
ccatcggatcctcaactca

SEQ ID NO: 158 - DNA, 20 bp, Artificial Sequence
  Description of Artificial Sequence Synthetic primer
ccatcggatcctcaactcac

SEQ ID NO: 159 - DNA, 35 bp, Artificial Sequence
  Description of Artificial Sequence Synthetic primer
ccgaacaacatggatagagcagttaaactatacaa

SEQ ID NO: 160 - DNA, 21 bp, Artificial Sequence
  Description of Artificial Sequence Synthetic primer
ccgcattacgtttggtggacc

SEQ ID NO: 161 - DNA, 21 bp, Artificial Sequence
  Description of Artificial Sequence Synthetic primer
cctctctcagaaacaaagtgt

SEQ ID NO: 162 - DNA, 23 bp, Artificial Sequence
  Description of Artificial Sequence Synthetic primer
ccttctttttacgtttactctcg

SEQ ID NO: 163 - DNA, 18 bp, Artificial Sequence
  Description of Artificial Sequence Synthetic primer
cgaggactgcagcgtaga

SEQ ID NO: 164 - DNA, 19 bp, Artificial Sequence
  Description of Artificial Sequence Synthetic primer
cgcgatcaaaacaacgtcg

SEQ ID NO: 165 - DNA, 23 bp, Artificial Sequence
  Description of Artificial Sequence Synthetic primer
cgtagacgctttgtccaaaatgc

SEQ ID NO: 166 - DNA, 24 bp, Artificial Sequence
  Description of Artificial Sequence Synthetic primer
cgtgcccagtgagcgaggactgca

SEQ ID NO: 167 - DNA, 26 bp, Artificial Sequence
  Description of Artificial Sequence Synthetic primer
cgttcatccatagttgcctgactccc

SEQ ID NO: 168 - DNA, 25 bp, Artificial Sequence
  Description of Artificial Sequence Synthetic primer
ctaaacgaacaaactaaaatgtctg

SEQ ID NO: 169 - DNA, 35 bp, Artificial Sequence
  Description of Artificial Sequence Synthetic primer
ctaaatgggaatggggacccgaacaacatggatag

SEQ ID NO: 170 - DNA, 35 bp, Artificial Sequence
  Description of Artificial Sequence Synthetic primer
ctaggcataatgggagaatacagaggtacaccaag

SEQ ID NO: 171 - DNA, 22 bp, Artificial Sequence
  Description of Artificial Sequence Synthetic primer
ctaggtttcaaactttacttgc

SEQ ID NO: 172 - DNA, 35 bp, Artificial Sequence
  Description of Artificial Sequence Synthetic primer
ctagtgtacaagcagaaatggaacaagttgtggag

SEQ ID NO: 173 - DNA, 35 bp, Artificial Sequence
  Description of Artificial Sequence Synthetic primer
ctcacttctctagtgtagtattgggcaatgctgct

SEQ ID NO: 174 - DNA, 30 bp, Artificial Sequence
  Description of Artificial Sequence Synthetic primer
ctcagattcaactggcagtaaccagaatgg

SEQ ID NO: 175 - DNA, 35 bp, Artificial Sequence
  Description of Artificial Sequence Synthetic primer
ctcagattcaactggcagtaaccagaatggagaac

SEQ ID NO: 176 - DNA, 25 bp, Artificial Sequence
  Description of Artificial Sequence Synthetic primer
ctctagtgtagtattgggcaatgct

SEQ ID NO: 177 - DNA, 18 bp, Artificial Sequence
  Description of Artificial Sequence Synthetic primer
ctgtcacggccaatgtta

SEQ ID NO: 178 - DNA, 19 bp, Artificial Sequence
  Description of Artificial Sequence Synthetic primer
cttctcaacgtgccactcc

SEQ ID NO: 179 - DNA, 34 bp, Artificial Sequence
  Description of Artificial Sequence Synthetic primer
gaagagggagacaattagactggccacggaagaa

SEQ ID NO: 180 - DNA, 20 bp, Artificial Sequence
  Description of Artificial Sequence Synthetic primer
gaccccaaaatcagcgaaat

SEQ ID NO: 181 - DNA, 35 bp, Artificial Sequence
  Description of Artificial Sequence Synthetic primer
gacgctgtgacatcaaggacctgcctaaagaaatc

SEQ ID NO: 182 - DNA, 35 bp, Artificial Sequence
  Description of Artificial Sequence Synthetic primer
gactcaatttcctcacttctctagtgtagtattgg

SEQ ID NO: 183 - DNA, 24 bp, Artificial Sequence
  Description of Artificial Sequence Synthetic primer
gacttgtggagacagccgctcacc

SEQ ID NO: 184 - DNA, 35 bp, Artificial Sequence
  Description of Artificial Sequence Synthetic primer
gagcttcgcagcgtgtagcaggtgactcaggtttt

SEQ ID NO: 185 - DNA, 35 bp, Artificial Sequence
  Description of Artificial Sequence Synthetic primer
gattatcaccagaagagggagacaattagactggc

SEQ ID NO: 186 - DNA, 18 bp, Artificial Sequence
  Description of Artificial Sequence Synthetic primer
gcacttgccagttgcatg

SEQ ID NO: 187 - DNA, 30 bp, Artificial Sequence
  Description of Artificial Sequence Synthetic primer
gcggtgtttgcagatttggacctgcgagcg

SEQ ID NO: 188 - DNA, 24 bp, Artificial Sequence
  Description of Artificial Sequence Synthetic primer
gctgcaatattgttaacgtgagtc

SEQ ID NO: 189 - DNA, 25 bp, Artificial Sequence
  Description of Artificial Sequence Synthetic primer
gctgcaatattgttaacgtgagtct

SEQ ID NO: 190 - DNA, 19 bp, Artificial Sequence
  Description of Artificial Sequence Synthetic primer
gctggcctaggcataatgg

SEQ ID NO: 191 - DNA, 20 bp, Artificial Sequence
  Description of Artificial Sequence Synthetic primer
gctggtgctgcagcttatta

SEQ ID NO: 192 - DNA, 19 bp, Artificial Sequence
  Description of Artificial Sequence Synthetic primer
gctttgtccaaaatgccct

SEQ ID NO: 193 - DNA, 35 bp, Artificial Sequence
  Description of Artificial Sequence Synthetic primer
ggaaccattacagatgctgtagactgtgcacttga

SEQ ID NO: 194 - DNA, 35 bp, Artificial Sequence
  Description of Artificial Sequence Synthetic primer
ggaagagacaggtacgttaatagttaatagcgtac

SEQ ID NO: 195 - DNA, 18 bp, Artificial Sequence
  Description of Artificial Sequence Synthetic primer
ggaccccaaaatcagcga

SEQ ID NO: 196 - DNA, 35 bp, Artificial Sequence
  Description of Artificial Sequence Synthetic primer
ggacccgaacaacatggatagagcagttaaactat

SEQ ID NO: 197 - DNA, 20 bp, Artificial Sequence
  Description of Artificial Sequence Synthetic primer
ggagaatacagaggtacacc

SEQ ID NO: 198 - DNA, 21 bp, Artificial Sequence
  Description of Artificial Sequence Synthetic primer
ggcactattctgaccagaccg

SEQ ID NO: 199 - DNA, 19 bp, Artificial Sequence
  Description of Artificial Sequence Synthetic primer
ggcagtaaccagaatggag

SEQ ID NO: 200 - DNA, 35 bp, Artificial Sequence
  Description of Artificial Sequence Synthetic primer
ggcggtgtttgcagatttggacctgcgagcgggtt

SEQ ID NO: 201 - DNA, 35 bp, Artificial Sequence
  Description of Artificial Sequence Synthetic primer
gtactgctgcaatattgttaacgtgagtcttgtaa

SEQ ID NO: 202 - DNA, 22 bp, Artificial Sequence
  Description of Artificial Sequence Synthetic primer
gtgaaatggtcatgtgtggcgg

SEQ ID NO: 203 - DNA, 21 bp, Artificial Sequence
  Description of Artificial Sequence Synthetic primer
gtgacatcaaggacctgccta

SEQ ID NO: 204 - DNA, 22 bp, Artificial Sequence
  Description of Artificial Sequence Synthetic primer
gtgacatcaaggacctgcctaa

SEQ ID NO: 205 - DNA, 19 bp, Artificial Sequence
  Description of Artificial Sequence Synthetic primer
gtggagacagccgctcacc

SEQ ID NO: 206 - DNA, 19 bp, Artificial Sequence
  Description of Artificial Sequence Synthetic primer
gtggggcgcgatcaaaaca

SEQ ID NO: 207 - DNA, 35 bp, Artificial Sequence
  Description of Artificial Sequence Synthetic primer
gtgtgcgtactgctgcaatattgttaacgtgagtc

SEQ ID NO: 208 - DNA, 18 bp, Artificial Sequence
  Description of Artificial Sequence Synthetic primer
gtgttcacgctcaccgtg

SEQ ID NO: 209 - DNA, 35 bp, Artificial Sequence
  Description of Artificial Sequence Synthetic primer
gttaggacacgctagtgtacaagcagaaatggaac

SEQ ID NO: 210 - DNA, 18 bp, Artificial Sequence
  Description of Artificial Sequence Synthetic primer
gttcaccgctctcactca

SEQ ID NO: 211 - DNA, 36 bp, Artificial Sequence
  Description of Artificial Sequence Synthetic primer
gttgctaaacttgttgctactgatgatcttacagtg

SEQ ID NO: 212 - DNA, 35 bp, Artificial Sequence
  Description of Artificial Sequence Synthetic primer
gtttcggaagagacaggtacgttaatagttaatag

SEQ ID NO: 213 - DNA, 30 bp, Artificial Sequence
  Description of Artificial Sequence Synthetic primer
taatggaccccaaaatcagcgaaatgcacc

SEQ ID NO: 214 - DNA, 20 bp, Artificial Sequence
  Description of Artificial Sequence Synthetic primer
tagactgtgcacttgaccct

SEQ ID NO: 215 - DNA, 20 bp, Artificial Sequence
  Description of Artificial Sequence Synthetic primer
tagtgtacaagcagaaatgg

SEQ ID NO: 216 - DNA, 35 bp, Artificial Sequence
  Description of Artificial Sequence Synthetic primer
tatagtgctattggcaaaattcaagactcactttc

SEQ ID NO: 217 - DNA, 26 bp, Artificial Sequence
  Description of Artificial Sequence Synthetic primer
tatgctaatagtgtttttaacatttg

SEQ ID NO: 218 - DNA, 19 bp, Artificial Sequence
  Description of Artificial Sequence Synthetic primer
tcagcacctcatggtgtag

SEQ ID NO: 219 - DNA, 20 bp, Artificial Sequence
  Description of Artificial Sequence Synthetic primer
tcatcttatgtccttccctc

SEQ ID NO: 220 - DNA, 18 bp, Artificial Sequence
  Description of Artificial Sequence Synthetic primer
tcatgacgttcgtgttgt

SEQ ID NO: 221 - DNA, 19 bp, Artificial Sequence
  Description of Artificial Sequence Synthetic primer
tcattcgtttcggaagaga

SEQ ID NO: 222 - DNA, 22 bp, Artificial Sequence
  Description of Artificial Sequence Synthetic primer
tcctcaactcactcttcgagcg

SEQ ID NO: 223 - DNA, 20 bp, Artificial Sequence
  Description of Artificial Sequence Synthetic primer
tcctcgtgaaggtgtctttg

SEQ ID NO: 224 - DNA, 24 bp, Artificial Sequence
  Description of Artificial Sequence Synthetic primer
tcctcgtgaaggtgtctttgtttc

SEQ ID NO: 225 - DNA, 35 bp, Artificial Sequence
  Description of Artificial Sequence Synthetic primer
tcctggtgattcttcttcaggttggacagctggtg

SEQ ID NO: 226 - DNA, 35 bp, Artificial Sequence
  Description of Artificial Sequence Synthetic primer
tcgtggacatcttcgtattgctggacaccatctag

SEQ ID NO: 227 - DNA, 24 bp, Artificial Sequence
  Description of Artificial Sequence Synthetic primer
tctagtgtagtattgggcaatgct

SEQ ID NO: 228 - DNA, 30 bp, Artificial Sequence
  Description of Artificial Sequence Synthetic primer
tctgataatggaccccaaaatcagcgaaat

SEQ ID NO: 229 - DNA, 35 bp, Artificial Sequence
  Description of Artificial Sequence Synthetic primer
tctgataatggaccccaaaatcagcgaaatgcacc

SEQ ID NO: 230 - DNA, 35 bp, Artificial Sequence
  Description of Artificial Sequence Synthetic primer
tgactcctggtgattcttcttcaggttggacagct

SEQ ID NO: 231 - DNA, 22 bp, Artificial Sequence
  Description of Artificial Sequence Synthetic primer
tgcacttttatctactgatggt

SEQ ID NO: 232 - DNA, 35 bp, Artificial Sequence
  Description of Artificial Sequence Synthetic primer
tggctaaagacaagaccaatcttgtcacctctgac

SEQ ID NO: 233 - DNA, 35 bp, Artificial Sequence
  Description of Artificial Sequence Synthetic primer
tgtgctcaagtattgagtgaaatggtcatgtgtgg

SEQ ID NO: 234 - DNA, 25 bp, Artificial Sequence
  Description of Artificial Sequence Synthetic primer
tgtgggttatcttcaacctaggact

SEQ ID NO: 235 - DNA, 22 bp, Artificial Sequence
  Description of Artificial Sequence Synthetic primer
tgtgtgcgtactgctgcaatat

SEQ ID NO: 236 - DNA, 20 bp, Artificial Sequence
  Description of Artificial Sequence Synthetic primer
tgttgctacatcacgaacgc

SEQ ID NO: 237 - DNA, 24 bp, Artificial Sequence
  Description of Artificial Sequence Synthetic primer
tgttgctacatcacgaacgctttc

SEQ ID NO: 238 - DNA, 30 bp, Artificial Sequence
  Description of Artificial Sequence Synthetic primer
tgtttgcagatttggacctgcgagcgggtt

SEQ ID NO: 239 - DNA, 19 bp, Artificial Sequence
  Description of Artificial Sequence Synthetic primer
ttaggacacgctagtgtac

SEQ ID NO: 240 - DNA, 34 bp, Artificial Sequence
  Description of Artificial Sequence Synthetic primer
ttatgaaccacaaatcattactacacacaacaca

SEQ ID NO: 241 - DNA, 35 bp, Artificial Sequence
  Description of Artificial Sequence Synthetic primer
ttattacaaattgggagcttcgcagcgtgtagcag

SEQ ID NO: 242 - DNA, 18 bp, Artificial Sequence
  Description of Artificial Sequence Synthetic primer
ttcaccgctctcactcaa

SEQ ID NO: 243 - DNA, 25 bp, Artificial Sequence
  Description of Artificial Sequence Synthetic primer
ttctatagattagctaatgagtgtg

SEQ ID NO: 244 - DNA, 24 bp, Artificial Sequence
  Description of Artificial Sequence Synthetic primer
ttggcaaaattcaagactcacttt

SEQ ID NO: 245 - DNA, 35 bp, Artificial Sequence
  Description of Artificial Sequence Synthetic primer
tttatgaaccacaaatcattactacacacaacaca

SEQ ID NO: 246 - DNA, 33 bp, Artificial Sequence
  Description of Artificial Sequence Synthetic primer
tttggtggaccctcagattcaactggcagtaac

(19) United States
(12) Patent Application Publication
(10) Pub. No.: US 2022/0213531 A1
(43) Pub. Date: Jul. 7, 2022

(54) MOLECULAR ELECTRONIC SENSORS FOR GENETIC ANALYSIS BY HYBRIDIZATION

(71) Applicant: Roswell Biotechnologies, Inc., San Diego, CA (US)

(72) Inventors: Carl Fuller, San Diego, CA (US); Barry Merriman, San Diego, CA (US); Paul W. Mola, San Diego, CA (US); Hadi Abderrahim, San Diego, CA (US); Andrew Hodges, San Diego, CA (US)

(73) Assignee: Roswell Biotechnologies, Inc., San Diego, CA (US)

(21) Appl. No.: 17/476,424

(22) Filed: Sep. 15, 2021

Related U.S. Application Data
(63) Continuation of application No. PCT/US21/24730, filed on Mar. 29, 2021.
(60) Provisional application No. 63/003,653, filed on Apr. 1, 2020.
(60) Provisional application No. 63/001,249, filed on Mar. 27, 2020.

(51) Int. Cl. C12Q 1/6825; C12Q 1/70; C12Q 1/6834

(52) U.S. Cl. CPC C12Q 1/6825; C12Q 1/6834; C12Q 1/701

(57) ABSTRACT

A molecular electronics sensor capable of performing genetic analysis is described. In various embodiments, the sensor comprises spaced-apart electrodes, a bridge molecule coupled to the electrodes, and an oligonucleotide hybridization probe conjugated to the bridge molecule. The hybridization probe may comprise an oligonucleotide sequence complementary to a segment of a pathogen genome to be detected. In various aspects, a plurality of such sensors are disposed as an array of pixels on a CMOS chip. Sensors herein can be configured to detect a segment of SARS-CoV-2 genome in a bio-sample.

### CROSS-REFERENCE TO RELATED APPLICATIONS

This application is a continuation application of International Application Serial No. PCT/US2021/024730 filed Mar. 29, 2021, entitled “Molecular Electronic Sensors for Genetic Analysis by Hybridization” and claims priority to this '730 Application. The '730 Application claims priority to, and the benefit of, U.S. Provisional Patent Application Ser. No. 63/001,249 filed Mar. 27, 2020, entitled “Molecular Electronic Sensors for Genetic Analysis by Hybridization,” and U.S. Provisional Patent Application Ser. No. 63/003,653 filed Apr. 1, 2020, entitled “Molecular Electronic Sensors for Genetic Analysis by Hybridization.” Each of these disclosures are incorporated herein by reference in their entireties for all purposes.

### SEQUENCE LISTING

The instant application contains a Sequence Listing which has been submitted electronically in ASCII format and is hereby incorporated by reference in its entirety. Said ASCII copy, created on Jan. 20, 2022, is named ROS-09416-US-CON1_SL.txt and is 57,183 bytes in size.

### FIELD

The present disclosure generally relates to molecular electronic sensors, and in particular, to sensor devices configured with hybridization probes for analyzing genetic material, such as in the detection, monitoring, and diagnosis of infectious diseases.

### BACKGROUND

In the field of genetic analysis, it is important to be able to determine if a given sample of biological material contains a DNA or RNA segment of interest. Also important is species identification, where a sequence characteristic of a species is searched for within a sample. These analyses are important for the environmental monitoring of, and diagnosis of, infectious disease. For example, a DNA segment that identifies a pathogen, such as a parasite, bacteria or virus, can be looked for within a sample taken from the environment, or in a bio-sample from an animal or human that may be infected. This is especially important for the environmental surveillance and epidemiology of viral diseases with the potential for large scale, rapidly progressing infection or pandemics, such as COVID-19. It is also important in genetic analysis to look for known genetic variants that may occur relative to a give segment of DNA. This type of measurement is known as genotyping, and in various aspects comprises looking for known variants in humans and animals. In the context of pathogens, this often takes the form of identification of strains, which are defined by DNA or RNA variants relative to a reference genome sequence, or by the sequence differences between two genomes.

Also in the field of genetic analysis, it is important to be able to determine the concentration of a DNA or RNA segment of interest in a sample. One such example is in gene expression analysis, where the activity level or expression level of genes, represented in the form of messenger RNA, can be assessed in a sample. This is important, for example, in studying gene function, or in characterizing the pathology of cancers for research, diagnosis and treatment. Another such example is in Non-Invasive Pregnancy Testing (NIPT), which requires measurement of levels of non-maternal cell-free DNA fragments in blood samples. Another similar example is Liquid Biopsy, for early detection or recurrence monitoring of cancer, which may look to detect of the levels of known mutant sequences in blood samples. Another example is Comparative Genomic Hybridization (CGH), where the relative concentration of a segment of genomic DNA in a sample is used to detect genomic duplication or deletion events, both in diagnosing germline disease such as Down Syndrome (Trisomy 21), or in characterizing genomic alterations in cancers as a component of Precision Medicine for Oncology. Another example arises in the field called metagenomics, where the goal is to characterize complex populations of diverse organisms present in an environmental sample, such as a soil or water sample, by extracting and quantifying the abundance of different forms of genomic DNA present in the organisms in the sample. Of particular interest for health and disease is the special case of assessing microbiomes, such as gut microbiome, or oral microbiome, for the populations of bacteria present. For the purpose of quantifying such complex populations, one common approach is to use PCR to target a common “barcode of life” DNA segment that is present in all the organisms of interest, and has enough diversity to distinguish species and strains of interest, an in this approach, the focus becomes identifying and measuring the relative concentrations of these fragments.

Each of these and other aspects of genetic analysis would benefit from having new devices, systems and methods usable for rapid, low cost and accurate genetic analysis.

### SUMMARY

In accordance with various embodiments of the present disclosure, a molecular electronics sensor is described. In various embodiments, a molecular electronics sensor in accordance with the present disclosure is configured for genetic analysis. In various embodiments, a molecular electronics sensor configured for genetic analysis comprises a multiplexed array of individual sensors configured in subsets of sensor pixels having different configurations and detection abilities.

In various embodiments, a molecular electronics sensor configured for genetic analysis has the potential for faster and lower cost testing, testing that is simpler to perform, and enablement of distributed deployment or point-of-use deployment.

In various embodiments, these benefits are extended to the problems of genetic analysis that occur in the field of infectious disease, and especially viral disease, such as influenza, colds/respiratory viruses, including rhinoviruses and adenoviruses, AIDS virus/HIV, Ebola, Dengue, other hemorrhagic fever viruses, Hanta, Zika and West Nile Virus, SARS, MERS, and novel viruses with pandemic potential, such as COVID-19.

In various embodiments, the present disclosure provides an all-electronic, single molecule detector of DNA or RNA, methods for deployment of sensors in a semiconductor chip format, e.g., a CMOS chip device format, methods to prepare samples containing genetic material using primers for amplification, and methods to address genetic analysis problems in general.

In various embodiments, a sensor device comprises: a plurality of sensor pixels configured in an array on a semiconductor chip, the plurality of sensor pixels comprising at least a first subset of sensor pixels and a second subset of sensor pixels; wherein each sensor pixel in the plurality of sensor pixels comprises a molecular electronics sensor further comprising: a first electrode; a second electrode spaced-apart from the first electrode by a nanogap; a bridge molecule having a first end and a second end, the first end coupled to the first electrode and the second end coupled to the second electrode; and a hybridization probe having an oligonucleotide sequence conjugated to the bridge molecule; wherein each molecular electronics sensor in the first subset of sensor pixels includes a first hybridization probe comprising a first oligonucleotide sequence; and wherein each molecular electronics sensor in the second subset of sensor pixels includes a second hybridization probe comprising a second oligonucleotide sequence.

In various embodiments, each bridge molecule comprises a polypeptide.

In various embodiments, the first oligonucleotide sequence is complementary to a segment of a first pathogen genome.

In various embodiments, the first pathogen genome is SARS-CoV-2.

In various embodiments, the second oligonucleotide sequence is complementary to a segment of an expressed human gene. In various embodiments, the expressed human gene comprises a constitutively expressed human gene.

In various embodiments, the human gene is human RNase P gene.

In various embodiments, the first oligonucleotide sequence is complementary to a segment of a first pathogen genome and the second oligonucleotide sequence is complementary to a segment of a constitutively expressed human gene.

In various embodiments, the plurality of sensor pixels further comprises a third subset of sensor pixels, and wherein each molecular electronics sensor in the third subset of sensor pixels includes a third hybridization probe comprising a third oligonucleotide sequence.

In various embodiments, each bridge molecule in the plurality of sensor pixels comprises a polypeptide.

In various embodiments, the first oligonucleotide sequence is complementary to a segment of a first pathogen genome, the second oligonucleotide sequence is complementary to a segment of an expressed human gene, and the third oligonucleotide sequence is complementary to a segment of a second pathogen genome. In various embodiments, the expressed human gene comprises a constitutively expressed human gene.

In various embodiments, the first pathogen genome is SARS-CoV-2, the second pathogen genome is a strain of Influenza A, a strain of Influenza B, or RSV, and the human gene is human RNase P gene.

In various embodiments, the plurality of sensor pixels further comprises a third subset of sensor pixels and a fourth subset of sensor pixels, wherein each molecular electronics sensor in the third subset of sensor pixels includes a third hybridization probe comprising a third oligonucleotide sequence, and wherein each molecular electronics sensor in the fourth subset of sensor pixels includes a fourth hybridization probe comprising a fourth oligonucleotide sequence.

In various embodiments, each bridge molecule in the plurality of sensor pixels comprises a polypeptide.

In various embodiments, the first oligonucleotide sequence is complementary to a segment of a first pathogen genome, the second oligonucleotide sequence is complementary to a segment of an expressed human gene, the third oligonucleotide sequence is complementary to a segment of a second pathogen genome and the fourth oligonucleotide sequence is complementary to a segment of a third pathogen genome. In various embodiments, the expressed human gene comprises a constitutively expressed human gene.

In various embodiments, the first pathogen genome is SARS-CoV-2, the second pathogen genome is a strain of Influenza A or a strain of Influenza B, the third pathogen genome is RSV, and the human gene is human RNase P gene.

In various embodiments, the plurality of sensor pixels further comprises a third subset of sensor pixels, a fourth subset of sensor pixels, and a fifth set of sensor pixels, wherein each molecular electronics sensor in the third subset of sensor pixels includes a third hybridization probe comprising a third oligonucleotide sequence; wherein each molecular electronics sensor in the fourth subset of sensor pixels includes a fourth hybridization probe comprising a fourth oligonucleotide sequence; and wherein each molecular electronics sensor in the fifth subset of sensor pixels includes a fifth hybridization probe comprising a fifth oligonucleotide sequence.

In various embodiments, each bridge molecule in the plurality of sensor pixels comprises a polypeptide.

In various embodiments, the first oligonucleotide sequence is complementary to a segment of a first pathogen genome, the second oligonucleotide sequence is complementary to a segment of an expressed human gene, the third oligonucleotide sequence is complementary to a segment of a second pathogen genome, the fourth oligonucleotide sequence is complementary to a segment of a third pathogen genome, and the fifth oligonucleotide sequence is complementary to a segment of a fourth pathogen genome. In various embodiments, the expressed human gene comprises a constitutively expressed human gene.

In various embodiments, the first pathogen genome is SARS-CoV-2, the second pathogen genome is a strain of Influenza A, the third pathogen genome is a strain of Influenza B, the fourth pathogen genome is RSV, and the human gene is human RNase P gene.

In various embodiments, each molecular electronics sensor in the first subset of sensor pixels further comprises a first decoding probe bonded to either the bridge molecule or the first hybridization probe in the molecular electronics sensor; each molecular electronics sensor in the second subset of sensor pixels further comprises a second decoding probe bonded to either the bridge molecule or the second hybridization probe in the molecular electronics sensor; each molecular electronics sensor in the third subset of sensor pixels further comprises a third decoding probe bonded to either the bridge molecule or the third hybridization probe in the molecular electronics sensor; each molecular electronics sensor in the fourth subset of sensor pixels further comprises a fourth decoding probe bonded to either the bridge molecule or the fourth hybridization probe in the molecular electronics sensor; and each molecular electronics sensor in the fifth subset of sensor pixels further comprises a fifth decoding probe bonded to either the bridge molecule or the fifth hybridization probe in the molecular electronics sensor.

In various embodiments, a method of detecting a target oligonucleotide sequence in a bio-sample comprises: providing a sensor device comprising a plurality of sensor pixels configured in an array on a semiconductor chip, the plurality of sensor pixels comprising at least a first subset of sensor pixels; wherein each sensor pixel in the plurality of sensor pixels comprises a molecular electronics sensor further comprising: a first electrode; a second electrode spaced-apart from the first electrode by a nanogap; a bridge molecule having a first end and a second end, the first end coupled to the first electrode and the second end coupled to the second electrode; and a hybridization probe having an oligonucleotide sequence conjugated to the bridge molecule; wherein each molecular electronics sensor in the first subset of sensor pixels includes a first hybridization probe comprising a first oligonucleotide sequence capable of hybridizing to the target oligonucleotide sequence; initiating at least one of a voltage or a current through each sensor pixel in the plurality of sensor pixels; exposing the plurality of sensor pixels to the bio-sample; and measuring electrical signals from the first subset of sensor pixels as the target oligonucleotide sequence and the first hybridization probe engage in hybridization, wherein the electrical signals provide a signature indicating the target oligonucleotide sequence is present in the bio-sample.

In various embodiments, the method further comprises amplifying at least the target oligonucleotide sequence prior to exposure of the plurality of sensor pixels to the bio-sample. In various embodiments, the amplifying comprises a Polymerase Chain Reaction (PCR).

In various embodiments, the target oligonucleotide sequence comprises a segment from a genome of a first pathogen.

In various embodiments, the first pathogen is SARS-CoV-2.

In various embodiments, the plurality of sensor pixels further comprises a second subset of sensor pixels, wherein each molecular electronics sensor in the second subset of sensor pixels includes a second hybridization probe comprising a second oligonucleotide sequence capable of hybridizing to a segment of an expressed human gene. In various embodiments, the expressed human gene comprises a constitutively expressed human gene.

In various embodiments, the method further comprises confirming the bio-sample is of human origin by measuring electrical signals from the second subset of sensor pixels as the segment of an expressed human gene and the second hybridization probe engage in hybridization, wherein the electrical signals provide a signature indicating the segment of an expressed human gene is present in the bio-sample. In various embodiments, the expressed human gene comprises a constitutively expressed human gene.

In various embodiments, the human gene is human RNase P gene.

### BRIEF DESCRIPTION OF THE DRAWING FIGURES

The subject matter is pointed out with particularity and claimed distinctly in the concluding portion of the specification. A more complete understanding, however, may best be obtained by referring to the detailed description and claims when considered in connection with the following drawing figures:

FIG. 1 illustrates a general embodiment of a molecular electronics sensor circuit with applied voltage and measured current;

FIG. 2 illustrates the general processes of single stranded DNA hybridization to form a double stranded duplex DNA, with the specific example of the single stranded sequence ATAGGACACCGTT (SEQ ID NO: 57) hybridizing with its complementary sequence TATCCTGTGGCAA (SEQ ID NO: 58) to form the “DNA DUPLEX” shown (SEQ ID NO: 59);

FIG. 3 illustrates the general concept of engaging a DNA hybridization probe of SEQ ID NO: 57 into a molecular electronics circuit by conjugating the 3′ end of the oligonucleotide to the bridge molecule 305;

FIG. 4 illustrates the concept of hybridization events generating sensor signals in the measured current, wherein a sensor having a hybridization probe of SEQ ID NO: 57 binding a target oligonucleotide having a sequence that includes complementary sequence SEQ ID NO: 58;

FIG. 5 illustrates the concept that when exposed to a complex pool of DNA targets, the hybridization sensor having a probe with SEQ ID NO: 57 will generate stronger signals from proper hybridization binding events with nucleic acid having a segment of complementary sequence SEQ ID NO: 58, and weaker signals resulting from incomplete or off-target interactions with other nucleic acids (shown generically as rectangular strips without sequences. FIG. 5 discloses SEQ ID NOS 57-58, 58, and 58, respectively, in order of appearance);

FIG. 6 illustrates the concept that a single sensor bridge molecule could comprise multiple hybridization probes, wherein each probe may have a common sequence represented by SEQ ID NO: 57;

FIG. 7 illustrates the architecture of a CMOS chips sensor array device and pixel that can provide for chip-based deployment of multiple molecular electronic hybridization sensors;

FIG. 8 illustrates the concept of a CMOS chips sensor array device where each measurement pixels could provide for the monitoring of multiple molecular electronic sensor elements, allowing multiple sensors per pixel;

FIG. 9 illustrates the concept of how molecular electronics hybridization sensors can be fully deployed in applications in pathogen detection and monitoring;

FIG. 10 (including panels FIG. 10A, FIG. 10B and FIG. 10C) illustrates a sensor embodiment used in demonstration experiments of primer concentration effects;

FIG. 11 illustrates exemplary signal traces obtained from one pixel of a 16 k sensor pixel chip on which the sensor embodiment of FIG. 10 is deployed;

FIG. 12 (including panels FIG. 12A, FIG. 12B and FIG. 12C) illustrates an expanded portion of the signal trace in the primer binding phase of the sensor data shown in FIG. 11, and a histogram of the measurement values indicating the relative time spent in the “on” and “off” states of hybridization;

FIG. 13 illustrates a signal trace from a sensor on a 16 k pixel chip, for an experiment in which the concentration of target primer is serially raised from 10 nM to 100 nM to 1000 nM;

FIG. 14 (including panels FIG. 14A, FIG. 14B and FIG. 14C) illustrates expanded portions of signals traces obtained from the three concentration phases of the experiment of FIG. 13, along with histograms indicating the relative time spent in “on” versus “off” states of hybridization;

FIG. 15 illustrates the measured fraction of time spent in the “on” (bound) state (X icons for the three data points), versus primer concentration, for the experiment of FIG. 13, illustrating the relationship between target concentration and fraction of time bound. Also shown is a fit of these data points to an expected exponential decay curve;

FIG. 16 illustrates a sensor embodiment used in demonstration experiments on the impact of perfect matches versus mismatches in the primers;

FIG. 17 illustrates an example sensor signal pixel trace from a 16 k pixel chip, from an experiment that serially exposed the sensor to different lengths of target primer;

FIG. 18 illustrates expanded regions of the signal from the data of FIG. 17, along with calculated values of primer length, melting point (Tₘ), duration of “on” events, and “off”-rate;

FIG. 19 (including panels FIG. 19A and FIG. 19B) illustrates the perfect match primer for the hybridization probe, and various mismatch primers with from 1 to 7 mismatches to the hybridization probe;

FIG. 20 illustrates expanded portions of signal traces from perfect match, single mismatch, and triple mismatch primers, and the related measured “on” times and “off”-rates;

FIG. 21 (including panels FIG. 21A, FIG. 21B, FIG. 21C and FIG. 21D) illustrates different example embodiments for linking decoding probe hybridization targets to the primary hybridization probe;

FIG. 22 (including panels FIG. 22A, FIG. 22B, FIG. 22C and FIG. 22D) illustrates alternative embodiments for conjugating the hybridization probe into a molecular electronics sensor, representing diverse attachment options;

FIG. 23 illustrates alternative embodiments for conjugating the hybridization probe into a molecular electronics sensor, by use of a complexing molecule;

FIG. 24 illustrates use of probe secondary structure, optionally in conjugation with a signal enhancing group, in a molecular electronics hybridization sensor;

FIG. 25 illustrates use of probe hairpin secondary structure, optionally in combination with a signal enhancing group, in a molecular electronics hybridization sensor;

FIG. 26 illustrates use of probe hairpin secondary structure, with mismatches, as well as a bridge oligo secondary structure, optionally in combination with a signal enhancing group, in a molecular electronics hybridization sensor;

FIG. 27 illustrates use of probe secondary structure with a mismatched protection strand, to create a parallel bridge structure, optionally in combination with a signal enhancing group, in a molecular electronics hybridization sensor;

FIG. 28 illustrates use of probe secondary structure with a mismatched protection strand, to create the primary bridge structure, optionally in combination with a signal enhancing group, in a molecular electronics hybridization sensor which opens the circuit upon hybridization;

FIG. 29 illustrates use of probe secondary structure with a mismatched protection strand, to create the primary bridge structure, optionally in combination with a signal enhancing group, in a molecular electronics hybridization sensor;

FIG. 30 illustrates use of the probe as a primary bridge structure in a molecular electronics hybridization sensor, in which hybridization produced a double-stranded bridge; and

FIG. 31 illustrates use of a hybridization probe in a molecular electronics hybridization sensor, in which the target strand is labeled with a signal enhancing group.

### DETAILED DESCRIPTION

The detailed description of exemplary embodiments makes reference to the accompanying drawings, which show exemplary embodiments by way of illustration and their best mode. While these exemplary embodiments are described in sufficient detail to enable those skilled in the art to practice the invention, it should be understood that other embodiments may be realized and that logical, chemical, and mechanical changes may be made without departing from the spirit and scope of the invention. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In various embodiments of the present disclosure, a molecular electronics sensor is disclosed. The molecular electronics sensor may be configured to perform genetic analysis, such as detection of particular DNA or RNA oligonucleotides and fragments. In various embodiments, a molecular electronics sensor herein is configured to identify the presence of and the identity of various strains of organisms in a sample, such as pathogens in a bio-sample.

In various embodiments, the molecular electronics sensor configured for genetic analysis comprises an array of sensor pixels. The array of sensor pixels may be multiplexed such that different analytes may be sensed in parallel. In various embodiments, the array may be multiplexed such that analog or digital signals are combined to enhance data quality.

In various embodiments, each sensor pixel comprises at least one biosensor. Each biosensor comprises a first nanoelectrode, a second nanoelectrode spaced apart from the first nanoelectrode by a nanogap, and a bridge molecule or bridging molecular complex electrically connected to the first and the second nanoelectrodes bridging the nanogap. In various embodiments, the bridging molecular complex comprises at least one DNA or RNA hybridization probe attached to a site on a single molecular wire bridging the electrodes and spanning the nanogap.

Definitions and Interpretations:

As used herein, the term “DNA” refers generally to not only to the formal meaning of deoxyribonucleic acid, but also, in contexts where it would makes sense, to the well-known nucleic acid analogs of DNA that are used throughout molecular biology and biotechnology, such as RNA, or RNA or DNA comprising modifications such as bases having chemical modifications, such as addition of conjugation groups at the 5′ or 3′ termini or on internal bases, or which includes nucleic acids analogues, such as peptide nucleic acid (PNA) or locked nucleic acid (LNA). DNA may generally refer to double stranded or single stranded forms in contexts where this makes sense, and unless specifically designated. In particular, when referring to hybridization and the probes and targets for a DNA molecule, they are interpreted in this broader sense of any of these analogs that undergo hybridization to form a bound duplex.

As used herein, the terms “hybridization” or “DNA hybridization” refer to the process by which a single stranded segment of DNA in solution pairs with its reverse complement sequence to form a duplex molecule via Watson-Crick base pairing, forming a double helical segment. It is understood here this includes the cases of DNA-RNA pairs forming, RNA-RNA pairs forming, and that such DNA could also include modified bases or nucleic acid analogs such as PNA or LNA. It is understood this pairing can occur between single strands of different length, such pairing occurring between the complementary segments of these longer sequences.

As used herein, the terms “complement,” “match,” “exact match,” and “reverse complement,” in relation to a given segment of single stranded DNA or RNA, all refer to another single strand of DNA or RNA that will hybridize properly with this strand to form a duplex with Watson-Crick base pairings (and base pairing U-A in the case of RNA-DNA or RNA-RNA pairings, in view that RNA has uracil (U) instead of thymine (T)) for the segment of interest.

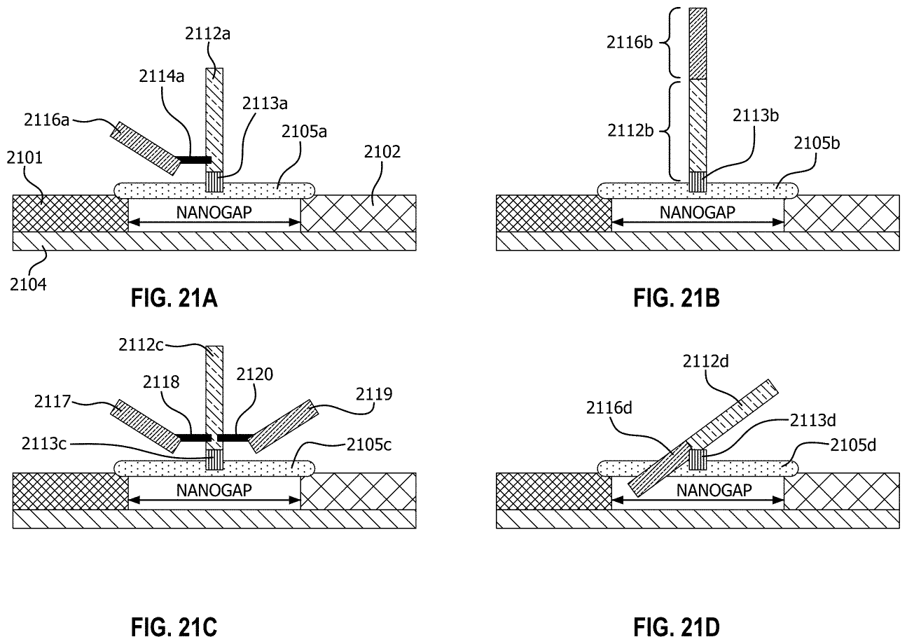

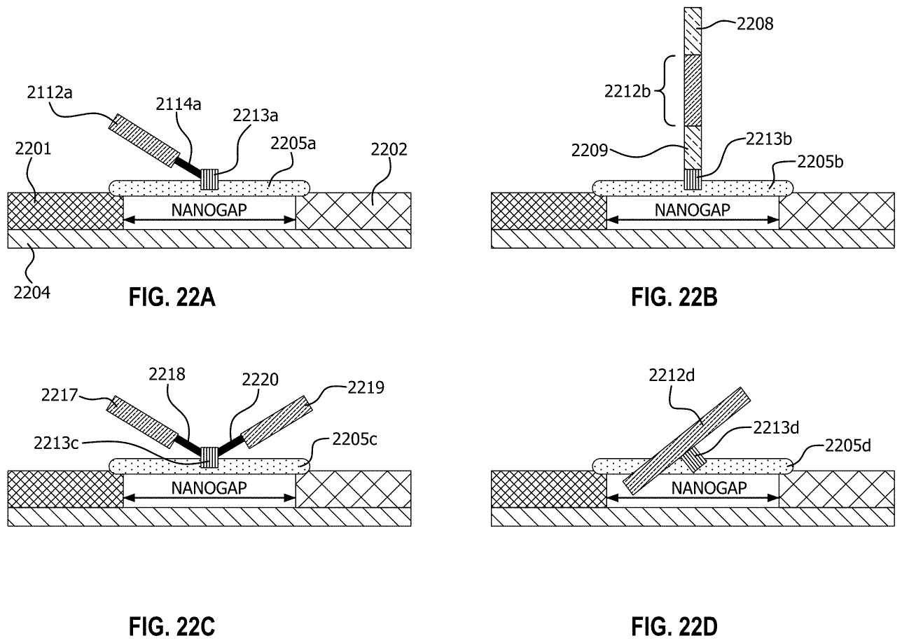

As used herein, the term “hybridization probe” refers to a molecule having a specific segment of DNA (or RNA) that is used to bind a complementary oligonucleotide strand of interest. Such a strand of interest may exist within a sample or complex pool of known or unknown DNA or RNA fragments, or a diverse set of oligonucleotides presented in a solution environment that allows for the hybridization reaction. A hybridization probe may also include a segment that provides a conjugation site to anchor the molecule in place in a sensor for exposure to a test sample solution. In context, the hybridization probe may refer to the single molecule of interest, or to a quantity of such molecules that all have the same sequence or different sequences. A hybridization probe in many instances may be a short segment of DNA, in the range of from about 10 to about 100 bases, but in general can be a DNA strand of any length. As used herein, the hybridization probe may generally refer to a DNA segment for which only a portion of it is used to hybridize to a target of interest, with other portions of the segment serving different purposes, such as spacers, segments comprising conjugation sites, segments intended to hybridize to other distinct targets, segments intended to bind DNA primers, or sites for binding of decoding probes use to produce location maps for sensor arrays on a chip, including segments that are sites for hybridization to targets that are decoding probes comprising DNA hybridization oligonucleotides, including such oligonucleotides used for combinatorial decoding, wherein oligonucleotides may be labelled or unlabeled with additional signaling groups to aid in decoding of sensor arrays. In various embodiments, a hybridization probe herein may be set forth as a probe functionalized for PCR in that any one of a dye molecule or quencher may be part of the oligonucleotide probe. In various embodiments, a PCR probe having functionalization may be anchored into a molecular electronics sensor by leaving the PCR probe with a free 5′-end or 3′-end, or having an internal position between the 5′ and 3′ ends capable of conjugation to a bridge molecule of a molecular electronics sensor. Thus, in various embodiments, a probe functionalized at both the 5′ and 3′ ends may still be used as a hybridization probe attached to a molecular electronics sensor since the probe may be functionalized with a suitable conjugation site somewhere between the 5′ and 3′ ends (e.g., as per the probe-bridge conjugation illustrated in FIGS. 21D and 22D where the bound probe could still have dye and/or quencher conjugation at the free 5′ and/or 3′-ends).

As used herein, the term “primer” refers to a single stranded DNA oligonucleotide that has a hybridization binding site on a single stranded DNA template molecule of interest, and the term “primer binding” refers to hybridization of this oligonucleotide to its target site. This term arises from the well-known process of priming a single strand for synthesis of the complementary strand by a polymerase enzyme. However, in the present context, primers and primer binding are merely an alternative way to refer to the process of an oligonucleotide DNA that binds to its complementary site via hybridization, in a context where the primer is typically a relative short segment, e.g., from about 6 to about 60 bases, and more commonly from about 12 to about 40 bases, or from about 16 to about 25 bases in length. In various embodiments, oligonucleotide sequences herein may provide multiple functions, in that a particular sequence may be used as any one of a forward primer, reverse primer, or hybridization probe in various PCR methods, or used as a hybridization probe conjugated to a molecular electronics sensor.

As used herein, the term “decoding probe” generally refers to any molecule whose binding and subsequent detection is used in a process of constructing a sensor map of where hybridization probes for different targets are located on a sensor pixel array. In this context, it is assumed there are a multiplicity of different types of DNA hybridization probes, having different target DNA as defined by the probe sequences, and that molecules of these types have been randomly assembled into a sensor pixel array, or otherwise placed in such a way that their location in the pixel array is unknown. It this context, each hybridization probe is assumed to have physically linked or connected to it, one or more binding sites configured to bind to one or more of the decoding probe molecules. The series of decoded probes are applied to such an array in series or in pooled form, allowed to bind to their specific targets on the hybridization probes, and the bound state is read out using the detectable signal generated by the binding probes. Such binding probes are single stranded DNA oligonucleotide hybridization probes, with hybridization targets on or linked to the DNA hybridization probes on the array. In various embodiments, the detectable signal in decoding is the electrical hybridization signal measurable by the sensor. In other embodiments, dye labels on such probes can be read out with an optical microscope imaging system. Other embodiments can use binding probes that are not based on DNA hybridization, such as aptamers or antibodies or libraries of small molecules.

As used herein, the term “combinatorial decoding” generally refers to any process of decoding the locations of hybridization probes on a sensor array, where a series of outcomes of multiple decoding probe binding reactions is used to generate a unique identifier “barcode” for the array of probes, which determines the hybridization probe identity and where such identification is not simply a one-to-one relation between individual decoding reactions and individual hybridization probes.

As used herein, the term “hybridization target” means DNA or RNA molecules which contain an oligonucleotide sequence complementary to a sequence on a DNA hybridization probe. Such a target could be the exact complementary strand to a hybridization probe, but in most cases the target will be a longer oligonucleotide strand that contains only a segment that is exactly complementary to the probe. In the context of targets with mismatches, molecules may match to the probe except at one or more bases. In the context of hybridization, a “perfect match” means a target sequence that correctly hybridizes to the probe sequence with no mis-paired bases, while a “mismatch” refers to a sequence that may bind to the probe sequence, but which has one or more mis-paired bases, i.e., bases not engaged in the standard Watson-Crick pairing found in natural double helix DNA-DNA or double helix DNA-RNA pairings. Such incomplete pairing will have reduced stability compared to the perfect match binding, which can be generally be used to discriminate perfect matches from mismatched forms, also known as cross-hybridization, in assay methodologies. A perfect match scenario results in a longer lived hybridization event, measured in time, versus a mismatch scenario having a shorter lived hybridization event measured in time. In various embodiments, a hybridization target comprises a segment of the genome from a pathogen, such as a short RNA segment from the SARS-CoV-2 genome, or from an Influenza A or Influenza B strain genome.

As used herein, the term “hybridization assay” refers to an assay or test that comprises the process of hybridization.

As used herein, the interchangeable terms “sample” or “bio-sample” refer to any material intended for testing. In various embodiments, a sample for testing may be in a solid or liquid physical form, and may also be packaged in some form of container, such as a tube, and/or reside in or on a carrier medium such as a swab or filter paper. In various embodiments, a sample may comprise animal or other tissue, cells, bodily fluids, excrement, food products, portions of plants, or any materials collected by a swab, an air filter or a water filter. Such samples may also be stably maintained with some form of preservative or stabilizing agents. The terms sample or bio-sample may refer to a material in its state as initially collected, or materials that have undergone various process steps, such as to extract or amplify DNA or RNA present, prior to being in a form suitable for introduction to a sensor device for analysis.

As used herein, the term “PCR” refers broadly to methods that use polymerase or reverse-transcriptase reactions to produce multiple copies of sequences from source DNA or RNA. In this context, the term “copies” may in general refer to single stranded reverse complements of segments of the source molecule, or single stranded exact copies of segments of the source molecule, or double stranded forms where one strand is identical to a segment of the source molecule. The term “copies” also may refer to the product arising from methods where an RNA template is converted to DNA molecules of the corresponding sequence, or a DNA template is converted to RNA molecules of the corresponding sequence. Such “PCR” methods in this context may include methods with linear amplification or exponential amplification, relative to time or cycle numbers. Such methods include those that use specific primers, or degenerate primers. Such methods also include isothermal reactions that occur in continuous time, or reactions that rely on thermal or chemical cycling. The “PCR” process may produce copies of specific target segments of the source DNA or RNA, as defined by specific primers, or may produce copies from many sites or random sites, as may result from degenerate primers. In particular, “PCR” in this context may refer to isothermal amplification methods that can be used to rapidly produce large amounts of DNA copy fragments from a source genome of RNA or DNA.

As used herein, the term “amplification” of DNA or RNA in a sample material refers to the use of PCR methods, such as recited above, to make copies of a source DNA or RNA. In various embodiments, particular primers may be used to amplify pathogen genetic material that may be present in a bio-sample prior to presenting the bio-sample to a sensor array chip in accordance with the present disclosure.

As used herein, the term “pathogen” refers to any disease causing agent that has a genome, such as parasites, fungi, viruses, or bacteria, or other single or multicellular organisms that cause disease.

As used herein, the term “strain” refers to genetic variants within a species, i.e., members of the same species that have genomes that differ in sequence.

As used herein, the term “molecular electronics” refers to electronic devices in which a single molecule or a single molecular complex is integrated as a component in an electronic circuit.

As used herein, the term “molecular electronics sensor” refers to a device that transduces molecular interactions into electronic signals, such as by using a single molecule or molecular complex integrated into an electrical circuit as the primary transduction mechanism and wherein the molecular interactions occur between the single molecule or molecular complex and target molecules provided in solution.

As used herein, the term “molecular complex” refers to an assemblage of a small number of molecules, such as only two, held together by chemical conjugation, bioconjugation, or covalent or non-covalent bonds, such that the assembly retains an assembled configuration or affiliation during a process of incorporating the molecular complex into an electrical circuit to provide a sensor, and during use of the resulting sensor in assays. In various embodiments, a small assemblage of molecules may comprise just two molecules, such as a DNA oligonucleotide hybridization probe chemically bound to a bridge molecule. In other embodiments, a molecular complex for use in a molecular electronics sensor may comprise from 2 to 10, from 10 to 100, or from 100 to 1000 molecules in the complex.

As used herein, the term “nanoelectrode” refers to an electrically conducting element having dimensions such as height, width and length of nanometer scale. In various embodiments, a length of a nanoelectrode may be substantially greater than both the height and width of the nanoelectrode such that an end portion of each nanoelectrode can be connected into a circuit. In various configurations, nanoelectrodes are disposed in pairs, wherein in each pair of nanoelectrodes a first nanoelectrode and a second nanoelectrode are spaced-apart by a nanoscale gap referred to as a nanogap. For simplicity, both nanoelectrodes and nanogaps may be called electrodes and gaps, respectively. In various embodiments, a nanoelectrode herein may comprise a metal such as Ag, Al, Au, Cr, Cu, Ni, Ga, Ti, Pt, Pd, Rb, Rh, Ru, or an alloy of these metals. In various embodiments, a contact may be disposed on a nanoelectrode, and the contact may be the same material as the nanoelectrode or a different material. For example, in various embodiments nanoelectrodes comprising Pt may each further comprise an Au nanoscale island in the form of nanopillars disposed at an end of the nanoelectrode.

As used herein, the term “bridge” or “bridge molecule” refers to a molecular wire or other electrically conducting molecule than may be used to make a conducting connection across a gap between spaced-apart nanoelectrodes in a pair of electrodes. Such molecules that function as bridges include, but are not limited to, double stranded DNA, peptide alpha helices, polypeptides having particular amino acid sequences, graphene nanoribbons, pilin filaments or bacterial nanowires, other multichain proteins or conjugates of multiple single-chain proteins, antibodies, carbon nanotubes e.g., single-walled carbon nanotubes (CNTs, SWCNTs), semiconductor layers such as transition metal dichalcogenides (TMD) or other semiconductor nanoribbons or nanowires, or conducting polymers such as polythiophene, poly(3,4-ethylenedioxythiophene (PEDOT) or other synthetic electrically conducting polymers. Such molecules may include attachment groups, i.e., functionality that provide for specific attachment to, and/or self-assembly to, nanoelectrodes or contacts such as islands or deposits thereon.

As used herein, the term “peptide” refers to any contiguous single chain of amino acids, wherein the amino acids are standard, non-standard or modified, or amino-acid analogs that engage in a peptide bond. In various embodiments, peptides herein may be in the range of 10 to 300 amino acids long, or 20 to 200 amino acids long.

As used herein, the terms “sequence identity” and “percent sequence identity,” in the context of two or more peptide sequences or oligonucleotide sequences, refer to two or more sequences or subsequences that have a specified percentage of amino acid or nucleotide base residues that are the same, when compared and aligned for maximum correspondence, as measured using a known sequence comparison algorithm for such comparisons or by visual inspection.

As used herein, the term “chip” refers to a semiconductor chip or a CMOS chip.

As used herein, the term “semiconductor chip” refers to an integrated circuit chip comprising semiconductor materials such as silicon or gallium, and which can be fabricated with techniques used in the semiconductor industry.

As used herein, the term “CMOS chip” refers to an integrated circuit chip fabricated using CMOS process techniques from the semiconductor industry. CMOS is an acronym for Complementary Metal-Oxide Semiconductor, and refers to a specific manufacturing process for making integrated circuit chips of the type most produced for processors, DRAM memory, and digital imager devices. As used herein, “CMOS chip” also refers to a device fabricated at the foundries that make such chips in industry, but which may also be postprocessed in accordance to the present disclosure, for example to add or to expose nanoelectrodes, suitably protection over such nanoelectrodes (e.g., dielectric layers), in order to configure the CMOS chip for use in molecule electronics sensor arrangements.

In various embodiments, the term “pixel” refers to a sensor circuit and/or a measurement circuit that is repeated throughout an array of such identical circuits disposed on a chip. A pixel may in context refer to just a measurement circuit, such as an electrical current meter measuring circuit, or may also include a sensor transducer element or elements affiliated with the circuit, which here are the molecular electronic components, i.e., molecules attached to nanoelectrodes. In various embodiments, a pixel may comprise at least one sensor, wherein each sensor comprises first and second nanoelectrodes separated by a nanogap and a bridge molecule bridging the gap and conjugated to at least one hybridization probe. In various embodiments, a pixel may comprise only one sensor circuit, i.e., only one pair of electrodes with its associated bridging molecular complex. In other embodiments, a pixel may comprise more than one molecular sensor, even a plurality of sensors. For definiteness, the term “measurement pixel” as used herein refers to a measurement circuitry of the pixel, and the term “sensor pixel” refers to a pixel circuit affiliated with a given sensor element. The origins of this term come from image sensors, where such pixels contain light sensing elements and measurement circuitry for capturing an element of a picture. In accordance with the present context, the term pixel is unrelated to light sensing or imaging, but rather the pixels disclosed herein are configured for sensing chemical interactions rather than light.

In various embodiments, the term “sensor” refers to a molecular electronics complex comprising a pair of nanoelectrodes, a bridge molecule and a hybridization probe conjugated to the bridge molecule, which is the primary transducer of interactions of the hybridization probes to electrical signals. In contexts where it makes sense, a sensor can also refer to this basic configuration plus the supporting current measurement circuitry, such as including the pixel circuits. “Sensor pixel” refers to the pixel circuitry that provides measurements to a particular sensor. In various embodiments, each sensor comprises a first nanoelectrode and a second nanoelectrode spaced-apart from the first nanoelectrode by a nanogap, and wherein a bridge molecule electrically connects the first and second nanoelectrodes together and bridges over the nanogap, and wherein at least one DNA or RNA hybridization probe is conjugated to a specific site along the bridge molecule.

As used herein, the term “signal group” or “signal enhancing group” refers to a chemical group that could be added to an oligonucleotide, such that the presence of this group complexed into a probe-bridge complex, versus dissociation from this complex, produces a detectable signal. In particular, such a group may be displaced from the critical position by target probe binding, or may be brought into proximity as a label on the target strand.

As used herein, the term “secondary structure” refers to the physical conformation that a DNA strand assumes in response to bonds it forms with itself or with other molecules. In particular, secondary structures include structures that form from hybridization between portions of a DNA molecule, or between two DNA molecules. Also included are structures that may result from the DNA strand interacting with the bridge molecule. Secondary structure can be induced by hybridization binding, or from other forms of binding.

As used herein, the term “multiplexed” refers to arrangements of individual sensor pixels for the purpose of sensing different analytes in parallel and/or for combining multiple analog or digital signals to augment the quality of data obtained from a sensor array. In various embodiments herein, a sensor array may be referred to as multiplexed in the sense that the array comprises a plurality of sensor pixels, each sensor pixel comprising at least one molecular sensor, and wherein each molecular sensor may be configured to hybridize with and thus detect either different analytes or the same analyte. Multiplexed sensor arrays herein, configured to detect different analytes in parallel, may further comprise switching arrangements that allow for individual pixels or groups of pixels to be queried as to the analyte or analytes they may have sensed. The multiplexed arrays may be organized in subsets of sensor pixels, wherein the summation of the subsets of sensor pixels equals the plurality of sensor pixels, and wherein each subset of sensor pixels is configured to detect a different analyte. Subsets may be geographically arranged on a chip, or may be interspersed.

### General Embodiments

In various embodiments, a molecular electronics sensor herein utilizes DNA or RNA hybridization in order to obtain benefits of testing speed, simplicity, robustness, and broad applicability that hybridization-based detection provides.

In various embodiments, a molecular electronics sensor herein, configured for genetic analysis, has the benefits of providing for faster testing, lower cost testing, lower cost test apparatus, and testing that is simpler to perform, and that also enables highly distributed deployment or point-of-use deployment of such testing systems, including mobile use and home use of such testing systems.

In various embodiments, an all-electronic, single molecule detector of DNA or RNA segments of interest is disclosed. In various embodiments, a molecular electronics sensor configured for genetic analysis can also obtain the concentration of DNA or RNA segments of interest. Further, the present disclosure provides methods for these sensors to be deployed in a semiconductor chip format, e.g., in a CMOS chip device format, and provides methods to perform highly multiplexed measurements on such chip devices in order to provide the benefits of low cost, rapid and portable testing, and the added benefit that such chip-based devices, systems and kits can be manufactured in extremely high volumes at low cost by leveraging existing manufacturing of the semiconductor industry. In various embodiments, the present disclosure provides methods by which these devices and systems can be used to address genetic analysis problems of importance specifically in the areas concerning the diagnosis and treatment of a disease.

In various embodiments, a DNA or RNA hybridization probe, which is a short piece of single stranded DNA or RNA, is suitably attached to a bridge molecule that spans the nanogap between two spaced-apart nanoelectrodes, whereby the bridge molecule is suitably and electrically attached to each. A voltage is applied across the electrodes, which is accompanied by current flow through the bridge molecule. This current can be measured versus time. When this molecular electronics sensor is exposed to a sample of DNA or RNA in solution, if the hybridization probe encounters and binds to its exact match target, a distinguishable signal is produced in the measured current. This signal provides primary detection of the target being present in the sample. If the exact match target is not encountered, there is no such distinguishable signal. This provides for the primary detection of targets of interest in a sample. In this configuration, a molecular electronics single molecule hybridization sensor detects the presence or absence of a target DNA or RNA molecule of interest in a sample.

In various embodiments, the detectible signal corresponding to a hybridization event comprises a series of spikes seen in the current measured over time, corresponding to target DNA or RNA binding to the hybridization probe (the “on” state), and then coming back off of the probe (the “off” state). This binding/unbinding is expected hybridization behavior, as the binding is reversible. Further, the rate of binding “on” is influenced by the concentration of the target DNA or RNA in solution, as well as the composition of the solution such as salt levels, divalent cation levels (such as Mg++, Mn++, etc.), pH, temperature, etc., and the rate of coming “off” is also dependent on these properties of the solution. In various embodiments, the binding/unbinding hybridization may be referred to as an “on-off event” having a particular and measurable rate.

In various embodiments, the properties of the observed signals, e.g., electrical current spikes, on-off rates, time between pulses, or the ratio of time on to time off, are relatable to the concentration of the target undergoing the hybridization event, and therefore provide a measure of concentration of the target of interest. In this way, a molecular electronics single molecule hybridization sensor can detect a concentration of a target DNA or RNA molecule of interest in a sample, including samples that may also comprise a complex pool of off-target oligonucleotide fragments.

In various embodiments, a perfect match between hybridization probe and DNA or RNA target will produce a detectible signal, whereas a single mismatch in a target DNA or RNA relative to the DNA or RNA probe in the sensor will produce a distinguishably altered signal. Further levels of mismatches will produce little or no distinguishable signals. In this way, the sensor signal can be used to distinguish targets that are a perfect match to the hybridization probe from even a single base mismatch. This provides sufficient sensitivity to perform genotyping and organism strain determination assays, which often require the ability to discriminate nucleic acid targets that differ by as little as a single base mismatch, or otherwise differ by just a few bases of mismatch, or insertions or deletions of one or a few bases. In particular, the identification of single nucleotide polymorphism (SNP) genotypes is possible, as these require single base discriminations.

In various embodiments, the present disclosure provides a method for SNP genotyping where, in a sample to be assessed, two or more sequence variants may be present, differing by one or more bases. To distinguish between two very closely related nucleic acid sequences, specific hybridization probes are made for each sequence variation contemplated and the probes are put into molecular electronic sensors as disclosed herein. From a primary bio-sample of interest, DNA or RNA is suitably amplified as necessary and/or purified as necessary using any of various means known in molecular biology, and the amplified/purified sample is applied to the sensors having probes for each of the two sequence variations. The sample may be applied separately, in different reaction volumes, or within one reaction volume applied to a device containing both sensors. Such a device could be a CMOS chip with both sensors present on the chip. By monitoring the signals from each sensor, one can determine if either, or both, or neither of the two variants are present in the sample, and this information can be used to determine a genotype for subsequent interpretation or to identify the presence of one or more specific strains of a pathogen. In various embodiments, analysis of a sample in this way determines the strain of a parasite, fungi, bacteria, or virus.

In various embodiments, a bridge molecule for any individual sensor may comprise any molecule that can serve as a conducting connection between the spaced-apart nanoelectrodes in a pair of electrodes. Such a conducting bridge molecule may comprise a double stranded DNA segment, an alpha-helical protein, a polypeptide having a desired amino acid sequence, a carbon nanotube, a graphene nanoribbon, a multi-chain protein such as a bacterial pilin filament or bacterial nanowire, or a conducting synthetic organic polymer such as PEDOT.

In various embodiments, such a bridge molecule includes a specific conjugation site to which the DNA or RNA hybridization probe conjugated. The conjugation between bridge molecule and hybridization probe may comprise covalent or noncovalent bonding, such as through any of many possible bioconjugation mechanisms. In various embodiments, the conjugation between hybridization probe and bridge molecule may comprise a click chemistry coupling, such as dibenzocyclooctyne-azide (DBCO-azide) or trans-cyclooctene-azide (TCO-azide), or other non-copper or copper click reactions, an 3-arylpropiolonitrile-thiol (APN-thiol) coupling, an amine-N-hydroxysuccinimide (amine-NHS) ester coupling, a biotin-avidin coupling, a peptide-tag based coupling such as Spy-Tag/Spy-Catcher, or an AviTag™ (GeneCopoeia, Inc.) or an aldehyde tag.

In various embodiment, a DNA or RNA hybridization probe for use herein may be between about 8 and about 200 bases in length, preferably between about 10 and about 100 bases in length, and more preferably from about 12 to about 60 bases in length. In applications requiring single base discrimination, such as in SNP genotyping or viral strain determinations, the probe length is preferably from about 12 to about 30 bases in length, or more preferably from about 15 to about 25 bases in length. In various embodiments, a DNA (or RNA) probe may also comprise other nucleic acids or nucleic acid analogs, such as RNA (or DNA), peptide nucleic acid (PNA) or locked nucleic acid (LNA), any of which may provide stronger binding or greater specificity of binding.

In various embodiments, a hybridization probe for use herein may comprise a fluorescent group, such as to be used for quality control in the fabrication of such sensor molecules or the finished sensors. In various embodiments, a hybridization probe may contain both a dye and a quencher group, provided there is at least one site on the hybridization probe, e.g., the 5′-end, the 3′-end, or a site internal in the sequence, for conjugation to a bridge molecule in each sensor.

In various embodiments, molecular sensors configured for genetic analysis in accordance with the present disclosure may be used for gene expression analysis of any cellular samples, and in general any application where methods such as DNA microarrays have been used for gene expression. In various embodiments, this would include gene expression applied to tumor tissue as might be used in cancer diagnostics.

In various embodiments, molecular sensors configured for genetic analysis in accordance with the present disclosure may be used for SNP genotyping in human, animal, or other cellular samples, and in general any application where methods such as DNA microarrays have been used for genotyping. In various embodiments, molecular sensors configured for genetic analysis can be used for SNP genotyping in humans.

In various embodiments, molecular sensors configured for genetic analysis in accordance with the present disclosure may be used for species identification, in particular for determining what species a given tissue sample is taken from, or in the identification of which bacteria or viruses are present in a given sample. In various embodiments, molecular sensors configured for genetic analysis can test environmental samples for the presence of a given virus, such as COVID-19. In various embodiments, a sample for testing may comprise a tissue sample from any of the common animal and insect vectors for viral transmission, such as bats, birds, rodents or mosquitoes. In various embodiments, a sample could comprise material previously filtered from air or water (e.g., taken from air or water filtration media), or material previously swabbed from a surface. In various embodiments, a sample may comprise a bio-sample taken from a human or animal subject, such as saliva, mucous, buccal swab, blood, sweat, urine, stool, or exhaled air.

In various embodiments, molecular sensors configured for genetic analysis in accordance with the present disclosure may be used for organism strain identification, in particular for determining what strain of bacteria or viruses are present in a given sample. In various embodiments, environmental samples may be tested for the presence of a given strain of a virus, such as COVID-19. The samples for testing could be the same as for the previous species identification application.

In various embodiments, molecular electronic sensors described herein are deployed as a sensor pixel array on a CMOS chip device.

In various embodiments, chip-based devices are deployed in a compact, low cost electronic instrument that is suitable for distributed use, field use, or point-of-care use.

In various embodiments, starting from a primary bio-sample acquired directly from a test subject or from the environment, some form of sample prep is required to prepare materials suitable for application to the sensor device for analysis. The primary sample may comprise tissue, saliva, mucous, buccal swab, blood, sweat, urine, stool, or exhaled air, or material filtered from air or water onto various types of media, or material swabbed from a surface. The sample prep could may result in a crude lysate containing DNA, or could be DNA purified from the sample by standard purification columns, filters, and procedures. In various embodiments, a prepared sample may be the result of applying any of the many forms of PCR amplification to a sample, which may comprise, for example, thermocycling or isothermal PCR using the primer sequences set forth herein. In various embodiments, sample prep is conducted by a self-contained sample prep device, or in other embodiments, a device integrated with the sensor platform, such as in the case of fully integrated point-of-use testing devices.

In various embodiments, a molecular wire used to bridge between spaced-apart nanoelectrodes in a pair of electrodes may have a length of from about 3 to about 10 nm, or from about 10 to about 100 nm, or from about 100 to about 1000 nm, or from about 1000 to about 10,000 nm.

In various embodiments, more than one hybridization probe is conjugated at specific sites on the bridging molecular wire, such probes being identical DNA probes or different DNA probes. The benefits of having multiple probes bonded to a common bridging molecular wire include, but are not limited to, increased signal, increased signal-to-noise ratio, robustness against failures in conjugation between probe and molecular wire, robustness against probes being physically inaccessible, or to allow for the ability to multiplex the sensor for detection of multiple targets, for greater robustness or breadth of detection. In various embodiments, multiple probes conjugated to a single molecule wire may number between 2 and about 10, between about 10 and about 100, or between about 100 and about 1000. Multiple probes on a single molecular wire bridge may be located so as to directionally point in similar orientations relative to the bridge, or may be located so as to point in different orientations away from the bridge. The spacing between each of the probes along a single molecular bridge can be in the range of about 1 to about 10 nm, or in the range of from about 10 nm to about 100 nm.

In various embodiments, molecular electronic sensors herein are deployed onto a CMOS chip device in the form of a sensor pixel array, where each pixel comprises at least one molecular sensor disclosed herein with nanoelectrodes appropriately configured and coupled into a circuit that provides for application of a voltage and measurement of a current, and for readout of the array of measured currents, and with the circuitry for the transfer of such data off-chip. In various embodiments, such data is digitized by on-chip ADC circuits before being transferred off-chip.

In various embodiments, more than one pair of nanoelectrodes are used per measurement pixel, and therefore more than one molecular electronic sensor exists per pixel so that one measurement pixel circuit may be used to monitor signals from multiple sensor devices (nanoelectrode pairs with bridge and hybridization probe molecule). In various embodiments, the measurement circuitry configured for applying voltages, measuring current, and reading out data from each pixel is applied serially in time to each sensor within the pixel, under control of transistor-based switches in the sensor circuit that can select any of the sensors individually or in groups for measurement. In this way, a number of N pixel circuits on chip can acquire measurements from a larger (and potentially much larger) number, M of sensors. This configuration has the benefit of greatly lower cost of chips deploying M sensors, versus having one pixel per sensor, because the size of the pixel is not increased in proportion to the number of nanoelectrode pairs in each pixel. In various embodiments, each pixel may have from about 2 to about 1024 sensors per pixel, and preferably in the range of from about 4 to about 128 sensors per pixel. In various embodiments, the chip array architecture is such that nearby pixels in the array share a common staging area where the many nanoelectrode pairs for these neighbors are all located and suitably electrically routed back to these adjacent pixel circuits. The use of such staging areas further improves the efficiency of circuit layouts and thereby improves pixel density, ultimately lowering chip costs for a given number of pixels and sensors.

Multiplex Probe Maps and Decoding Methods

In various embodiments, sensor arrays in which multiple distinct hybridization probe sensors are deployed on a single chip for the purpose of multiplex testing, interpretation of sensor data from the chip device benefits from a sensor map that shows which of the specific hybridization probes are present at the different pixel locations, or sensor locations within a pixel if multiple sensors are affiliated with each pixel. This allows the measured sensor data readout from the pixel array to be related to the particular probe target being assessed at each sensor. Such a sensor map may be produced by various techniques based on how the probe molecules are prepared and applied to assemble into the array. This map is referred to as the probe map for the sensor array or pixel array.

In various embodiments, a method for constructing a probe map of a sensor array comprises spatially controlled exposure of pixels to different solutions containing particular hybridization probes (or previously formed hybridization probe-bridge complexes) during sensor assembly, such that the probe map is determined by knowing which probe or molecular complex was applied to which pixels. This spatial control can be achieved by mechanically applying solution only to certain regions of the chip, instead of applying solution to the entire chip pixel array. In other embodiments, spatial control is achieved by applying a probe assembly solution to the entire chip array, but contemporaneously with a voltage-driven assembly process such that only electrically activated pixels attract and assemble the probe molecules or probe-bridge complexes thus applied into various sensor nanoelectrodes. In various embodiments, this can be accomplished by applying an appropriate voltage and/or polarity to electrodes to electrically configure electrodes to either attract or repel the probes or probe-bridge complexes in the local environment. For example, a positive voltage may be used to attract negative charges on DNA in solution, or a negative voltage may be used to repel such negatively charged DNA. This phenomena relies on the process of electrophoresis.

In other embodiments, an AC voltage may be used to selectively attract or repel the various probe or probe-bridge molecules, using the process of dielectrophoretic forcing. In particular, the solution containing a particular probe type or probe-bridge type destined for a particular target is applied to the solution for a short period of time and in a low concentration, such that diffusive transport is unlikely to deliver these molecules to bind to nanoelectrodes on the chip array. However, for the desired nanoelectrode locations of the molecules, an AC voltage of proper frequency and amplitude creates a dielectrophoretic force that will drive these molecules to concentrate near the electrode gaps of the intended sites in the form of localized concentrations, and allowing the electrodes to selectively bind the intended probes or probe-bridge complexes. The solution is then flushed away, and the next probe introduced in the presence of other dielectrophoretic forces at other electrodes. This procedure may be done for individual probes, or pools of distinct probe types, in which case their locations are restricted to a much smaller set of possible sites, yet probe type from the pool is still randomly distributed across electrodes within those site sets, and further location information would be required to complete the map to the individual sensor level. In various embodiments, the low concentrations of the probes used may be in the range of about 1 pM (pico-Molar) to about 100 nM (nano-molar), the exposure time used may be in the range of about 0.1 sec to about 100 sec, and the amplitude of applied voltage used to locally concentrate probe or probe-bridge molecules maybe be in the range of from about 0.1 V to about 10 V, and the frequency of AC modulation used may be in the range of from about 1 KHZ to about 100 MHZ.

In various embodiments, a probe map may be constructed by decoding hybridization probe locations by interpreting the results of special binding reactions, with special detectable probes (that are not necessarily DNA hybridization probes), designed to be able to locate or localize each specific hybridization probes type on the array. In various embodiments of a method for constructing a probe map of a sensor array, each distinct hybridization probe type (or probe-bridge complex) may be provided with one or more binding sites directly coupled to, or integrated into, the probe molecule (or probe-bridge complex), wherein the binding sites are capable of producing a unique, observable binding signature for that hybridization probe type in response to binding with the corresponding decoding probe molecules, and with such a signature being localizable down to the resolution of a specific probe site (i.e., nanoelectrode pair) or pixel, as required for the complete hybridization probe map. These decoding signatures can then be used to decode the pixel locations of the different types of probes post-assembly on the array. In this method, the hybridization probes (or probe-bridge complexes) are applied as a pool to the pixel array and allowed to randomly assemble onto the nanoelectrode pairs present on the pixels. After they are assembled into each of the pixels, the decoding probe molecules are applied to the array and the array is observed for the unique binding signatures localized to sensor sites, identifying which pixel sites have bound which hybridization probes.

In various embodiments, the observation of a decoding signature from a pixel array may be accomplished by monitoring electrical signals from the pixels produced in response to the decoding probe binding events. In various embodiments, these decoding probes are themselves DNA hybridization probes whose targets are affiliated with the hybridization probes, and their hybridization events also produce detectable signals in the sensor. The decoding scheme may require one or a multiplicity of such decoding hybridization targets affiliated with the hybridization probe. There are many ways such targets can be affiliated with a hybridization probe. For example, a DNA target of a decoding probe may be linked to a primary hybridization probe DNA by a linker molecule, and there may be two or more such linked targets. In general, any number of decoding targets can be affiliated with the probe to be encoded. For various other examples, a segment of a DNA strand containing the hybridization probe may also include another contiguous segment that is a target of the decoding probe and, in various embodiments, a number of such targets can be present in series, all encompassed within a single DNA strand. In various embodiments, conjugation of such a strand to the bridge molecule may be located at an interior part of the strand in order to allow both the hybridization probe segment, and the decoding target segments, to be similarly close in proximity to the bridge molecule to promote more sensitive detection of the decoding targets.

In other embodiments, these signature may comprise optical signatures produced from dye molecules by imaging the chip under white light or fluorescence conditions. In various embodiments, the dye molecules or fluorescent groups may comprise quantum dots configured on the decoding probes, wherein the optical signatures may be acquired by microscopic imaging of the chip under white light or fluorescent light, and widefield imaging or confocal scanning conditions. This procedure is sufficient to localize the optical signals to within approximately 1 micron, or one wavelength of the emitted light, of the location of the decoding probe itself, thus providing spatial resolution in the range of approximately 0.5 microns to about 1 micron. This is sufficient to localize the probes. If super-resolution imaging methods are employed, localization below the wavelength of light (the so-called diffraction limit) is possible, down to about 100 nm or down to about 20 nm.

In various embodiments, a probe map may be constructed by a process of “direct decoding” in which there are N total distinct decoding probe types D₁, D2, . . . D_{N}, whose targets are put in one to one fashion on N distinct hybridization probe types H₁, H₂, . . . , H_{N}, and where the decoding reactions are directly reacting D₁ against the array to observe all H₁ locations, reacting D₂ against the array to observe all H₂ locations, and so forth, up to reacting D_{N} against the array to observe H_{N} locations. These should have distinct sequence targets, having very low cross-hybridization between them. These probes are assembled randomly onto the sites on the chip array. Then, a series of N hybridization reactions is performed on the chip. In reaction i, decoding probe Dᵢ is hybridized to the array, and the detection readout of each sensor is recorded. Sensors where Dᵢ finds its target produce a detection signal, and this directly identifies which sensors have probe Pᵢ. In doing this process for reactions i=1 . . . N enables the decoding of the locations of all probes, resulting in the probe map. In various embodiments, this same method works in instances where instead of an electrical readout, optical labeling and imaging location is used to localize the decoding probes. For example, the decoding probes may be taken to be the targets of the hybridization probes in question, i.e., Dᵢ is the target of Hᵢ. This has the advantage that no extra target sequences need to be added to the hybridization probes to achieve decoding. However, the decoding probes Dᵢ may be chosen to have better physical hybridization properties than simple choice might afford, such as stronger on-target binding and weaker cross-hybridization with other decoding probe targets, or more uniform performance across the set.

In various embodiments, decoding methods that produce a probe map may be generally referred to as “combinatorial encoding and decoding.” In these methods, a series of decoding probe reactions are applied, and for each given probe site on the array, the series of detection/non-detection results from these reactions provide enough information, in aggregate, to uniquely determine the identity of the probe at the site. Several canonical embodiments of such combinatorial methods are discussed herein. It is understood that there are other variations, reformulations, and combinations of these that can be used as alternative decoding schemes for building a probe map. All such variations, reformulations, and combinations are meant to be encompassed by these example embodiments.

In various embodiments, the canonical combinatorial decoding embodiments provided may be described succinctly and efficiently as follows, wherein to achieve this, the assay to be performed, their order, and their outcomes are arranged and represented with 0/1 in ways that allow the direct relationship of decoding probe assay results to probe identification codes. For example, assuming there are N hybridization probes types H₁, H₂, . . . Hᵢ . . . H_{N} for which a location map is desired. In various embodiments of this method, there is provided a set of N distinct K-bit binary code strings {B₁, B₂, . . . Bᵢ, . . . B_{N}}, where these Bᵢ are various strings of length K, composed of the symbols “0” and “1”, such as, for example, might be the string B=“1001011” in a case where the length is K=7. The code Bᵢ is assigned to probe Hᵢ, for i=1 . . . N, and these codes will be used in physical encoding and decoding processes to identify this probe for the probe map on the array. Note that any such set of N strings will provide a valid encoding for the methods that follow, although special sets of such strings, as described herein, can also provide for the additional feature of error detection and correction in the decoding measurement process used in array assays. Also, note that as there are exactly 2^{K} distinct strings of length K, so it is required (in order to have enough such binary codes) that 2^{K}≥N. Indeed, for any K satisfying this requirement, various embodiments include the choice of any subset set of N strings from the master set of 2^{K} possibilities, and if N=2^{K}, an embodiment is simply to use all K-bit strings, listed in any order. Note that in these code assignments, if all the code strings {B₁, B₂ . . . Bᵢ . . . B_{N}} have the same binary digit in position j (i.e., the j^{th} digit is always 0, or always 1), this position is uninformative and can be eliminated from the strings, reducing their length K to K−1. This can be repeated to remove all such uninformative positions in the strings, so as to reduce the number of physical encoding probes required in the methods herein.

In various embodiments, there are N decoding probes and N hybridization probes as explained herein but the reactions comprise applying pools composed of a randomly chosen subset of half of the decoding probes. The outcome for each hybridization probe in such a pooled reaction is one bit of information regarding a target site of any probe in the pool. A series of K these reactions generates Kbits of decoding information, and, probabilistically, after K=Log₂[N] reactions, the expectation is that the hybridization probes are uniquely identified and mapped to the array. The advantage of this approach is to perform much fewer decoding hybridization reactions than for the direct approach, since for N>2 probe types, K is less than N, and much less for N>>2 (e.g., for N=64 probe types, K=6, for N=1024 probe types, K=10, and for N=1 million probe types, K=20).

In various embodiments of combinatorial decoding, there are 2K decoding probes with unique targets, denoted Dᵢⱼ, where i=0 or 1, and j=1 . . . K, i.e., D₀₁, D₁₁, D₀₂, D₁₂, . . . D₀₁, D₁₁, . . . D_{0K}, D_{1K}, linked to each hybridization probe is a series of K targets uniquely chosen by assigning one target from each decoding probe pair {D₀₁, D₁₁}, {D₀₂, D₁₂}, . . . , {D_{0K}, D_{1K}}. This encodes a total of 2^{K} hybridization probes, one for each unique binary pattern of D₀₁/D₁₁ selections. By performing these 2K hybridization reactions for the individual decoding probes, each hybridization probe on the array receives a K-bit identifier for whether Aᵢ or Bᵢ target the probe, i=1 . . . K. This K bit identifier uniquely determines the probe identity and map location. These decoding probes should have distinct target sequences, and preferably have low potential for cross-hybridization. For a probe Hᵢ, the associated physical encoding targets are taken to be the target DNA oligonucleotides of the encoding probes D(b1)1, D(b2)2, . . . , D(bK)K, where b1, b2, . . . , bK are the binary digits of the encoding string Bᵢ, i.e., b; is the j^{th} digit of string Bᵢ. These encoding probe target DNA oligos are then to be physically linked or affiliated with the physical hybridization probe oligonucleotides. Note that with the probes so encoded, that for any probe, Hᵢ, and a pair of encoding probes D₀ⱼ and D₁ⱼ, precisely one of these two probes will have its target on the physically encoded probe for Hᵢ. To achieve decoding of probe locations on the arrays, the series of 2K reactions trying to hybridize the individual encoding probes D₀₁, D₁₁, D₀₂, D₁₂, . . . D₀ⱼ, D₁ⱼ, . . . , D_{0K}, D_{1K} is performed, and for any probe site of the array, the outcome of these reactions is recorded by taking the trial of both D₀ⱼ and D₁ⱼ, and recording the outcome of these two reactions for the site as trial j=0 if D₀ⱼ is bound or trial j=1 if D₁ⱼ is bound, at the site in question. Then the complete binary string of trial outcomes for the site in question is succinctly written as (trial1)(trial2) . . . (trialk). As constructed in this process, this string will be identical to the code Bᵢ assigned to the hybridization probe Hᵢ that is in fact located at the site in question. In this way, for each site, the reaction results from the decoding probes are decoded to some precise Bᵢ code and related Hᵢ probe from the probe set. Therefore, the outcomes of this series of 2K decoding hybridizations across all sites on the array provides the code strings that identify and localizes in the array all occurrences of every probe H₁ . . . H_{N}. Thus, the probe map is constructed.

The advantage of this combinatorial decoding scheme is that the number of both the decoding probes, 2K, and decoding reactions, are much less than the number of hybridization probes, so this process does not require generation of a large number of individual decoding probes. For example, 12 decoding probes can decode 2⁶=64 hybridization probes, 20 decoding probes can decode 2¹⁰=1024 hybridization probes, and 40 decoding probes can decode 2²⁰=1 million hybridization probes. The disadvantage of this approach versus the other, is that here each hybridization probe must be linked to K decoding probe binding sites, and this can become limiting due to the size of steric hinderance constraints, since these sites must correspond to physical binding groups linked to the primary hybridization probe, such as K additional DNA oligonucleotide binding sites in the case where the decoding probes are DNA hybridization probes. In other embodiments, these two combinatorial methods, i.e., probe pooling and combinatorial encoding, can be combined. In the combined approach, there are a total of LK unique decoding probes, organized into groups of L probes, with K groups: {D₀₁, D₁₁, . . . , D_{L1}}, . . . , {D₀₁, . . . , D_{Li}}, {D_{0K}, . . . , D_{LK}}. The hybridization probes to be mapped each have K target sites, and at the i^{th} target site, a target for a probe in group i is assigned. There are a total of L^{K} unique assignment patterns, and thus this many hybridization probes can be decoded. The decoding reactions use the random 50% sub-pool method to decode the probes in each group so that each group requires Log₂[K] reactions for decoding with high statistical confidence. Thus, with a total of L×Log₂[K] pooled decoding reactions, with high statistical confidence, a total of L^{K} hybridization probes can be mapped. This allows for tradeoffs between the total number of decoding probes that must be produced, N=LK, the number of reactions that must be performed, L×Log₂[K], and the number of probes mappable, L^{K}. K=1 corresponds to pure decoding with 50% pools, and L=2 corresponds to the completely combinatorial encoding.

In various embodiments, physical encoding is performed more compactly. For the probe Hᵢ, the associated physical encoding targets are taken to be the target DNA oligonucleotides of the encoding probes D(b1)1, D(b2)2, . . . , D(bK)K, but probes are only physically tagged with the Dix targets. That is, probes are note tagged with any of the D0x targets when performing the decoding. Further, when performing this decoding above, only the K reactions of the probe D1x probes, D₁₁, D₁₂, . . . D_{Ij}, . . . , D_{1K} are applied. The results of these trial assays can be recorded as test j=1 if D₁ⱼ binds at a probe site, and test j=0 if it does not bind. In this case, the result string (test1)(test2) . . . (test j) . . . (testK) is the same binary string as recovered above in the previous embodiment, because above, if D₁ⱼ did bind, test j=1, as in the present method, and if D₁ⱼ did not bind, this is the same as D₀ⱼ binding, which also recorded as 0 above and in the present method. Thus, the same probe map decoding is achieved. It is a benefit of this embodiment that fewer physical target oligonucleotides need to be linked to each hybridization probes, and overall the method requires only half as many physical encoding probes to be produced, and their associated targets to be produced and linked to probes.

In various embodiments, reaction procedures and outcomes are efficiently encoded by 0/1 indicators that allow direct interpretation of decoding assay results for an unknown probe as the binary code identifying the probe. This method relies on reacting pools of decoding probes rather than individual probe reactions, but otherwise within a similar logical framework. In an example, hybridization probe types H₁ . . . H_{N}, are assumed present and assigned K-bit binary codes {B₁ . . . B_{N}}. There are then further provided the same number of N decoding probes that are hybridization probes denoted as D₁, . . . , D_{N}. These decoding probes should have distinct target sequences and preferably a low potential for cross-hybridization. The target of each Dᵢ is to be physically linked to the corresponding probe Hᵢ. A total of 2K pools of decoding probes P₀₁, P₁₁, P₀₂, P₁₂, . . . P₀₁, P₁ᵢ, . . . , P_{0K}, P_{1K}, are defined as follows. The members of pool P₀ⱼ are all probes Dᵢ for which bit j of code Bᵢ is 1, and similarly, the members of pool P₁ⱼ are all probes Dᵢ for which bit j of code Bᵢ is 0. Given these pool memberships, the corresponding physical probe pools are produced as equimolar mixtures of the decoding probe oligonucleotides for the pool. Under this construction, the result of reacting the physical pool of probes P₁ⱼ against a probe H; on the array will be a match if code B; has 1 in position j, and this outcome is to be recorded as trial j=1, while otherwise, if there is a 0 in position j off code Bᵢ, the match will instead occur for pool P₀ⱼ, and this outcome is denoted by trial j=0. With the outcome of all the K pooled reactions against the array recorded by the string (trial1)(trial2) . . . (trialK), then this string matches the code B; of the probe in question Hᵢ, and these series of reaction outcomes provides decoding of the probe identity. Thus, the results of reacting the 2K pools to the array decodes all occurrences of all probes on the array, and provides the required probe map. Note in one embodiment above, the Dᵢ could be taken as the targets of the Hᵢ, in which case no special linkage of targets to the Hᵢ is required. However, in general, other sets of {Dᵢ} could have more desirable hybridization properties of uniformity of Tₘ and low cross-hybridization potential, and better discrimination of perfect match signals from background.

In various embodiments, only the K pools P₁₁, P₁₂, . . . , P₁ⱼ, . . . P_{1K} are physically constructed, and these are reacted to the array in a series of K reactions. For each site on the array, the result is recorded as trial j=1 if hybridization was observed with pool P₁ⱼ, other 0 if it was not observed. The resulting string (trial1)(trial2) . . . (trialK) that encodes this outcome, is identical to the string in the above embodiments, and therefore this string provides the code string B; that identifies the probe Hᵢ. The results of reacting these K pools to the array, therefore, decodes all occurrences of all probes on the array, and provides the required probe map. This requires half as many pool constructions and hybridizations as the previous embodiments.

In various embodiments, decoding of a pixel array may comprise a combinatorial process, wherein a series of binding events, followed by signature detection events, is used to build up a meta-signature as a series of individual signatures. In various embodiments, decoding probes are themselves DNA hybridization probes that are added to the single stranded DNA oligonucleotide that also includes the primary hybridization probe sequence. In such embodiments, these decodable hybridization probes are longer single stranded DNA oligonucleotides that comprise a segment comprising the sequence of the hybridization probe for the target of interest, and other distinct sequence segments that are hybridization targets for the series of decoding hybridization probes. In such embodiments, the decoding process comprises a series of hybridization reactions, applying the decoding hybridization probes one at a time, and each time detecting for each pixel or nanoelectrode pair a signature indicating the presence or absence of the target sequence on the probe, and thereby building up a combinatorial signature to uniquely identify the probe type present at each pixel or nanoelectrode pair.

Embodiments for Error Detection and Correction in Probe Mapping

In various embodiments of decoding, any set of N binary K-bit strings {B₁, . . . , B_{N}} provides an encoding and decoding method. Within this framework, the specification of specific code word sets provide substantial benefits. For illustration of this point, note that in the combinatorial decoding schemes above, if the number of probes N=2^{K}, each and every K-bit binary string is then necessarily assigned a probe, in 1-to-1 fashion. However, in this minimal code length K scenario, if an error were made in measuring the code of a probe, one would produce the code of a different probe since all codes are used, and thus an incorrect decoding. Allowing a larger binary coding string length K than the minimum required allows for robustness against such errors. Specifically, it is possible that the set of binary codes {B₁, B₂, . . . , B_{K}} is chosen as a set that allows for error correction or detection, such that if a code string from this set were corrupted by one or more bit flipping errors, it is possible to determine that such corruption has occurred, and with some encodings, also to correct the error back to the uncorrupted state. This will provide for protection against errors that could be made in the decoding measurement process outlined above, in the form of a false detection of hybridization (error of 0→1), or missing a true hybridization (error of 1→0), so that such errors do not lead to incorrect or indeterminate decoding of probe identity. Many such error correction or error detection encodings are known to those skilled in binary data. In various embodiments, one such method is the use of binary strings that add one or more parity bits add the end of an initial given string, which provide power to detect or correct certain errors. Another embodiment comprises use of Hamming Codes and Hamming distance to detect and correct errors. In this class of methods, the assigned number N of code words must be only a small fraction of all possible binary codes of length K, and the precise code words are taken to have highly distinct bit sequences, such as, for example, this could be N randomly selected code words from all 2^{K}>>N. In such a case, if there is a corrupted code, it may be detected because it does not match any of the assigned codes, and it can be corrected back to the closest of the allowed assigned code strings, with closeness measured by the Hamming Distance (number of mismatches between the digits of two binary strings). This general technique always affords some power for error correction of at least limited number of bit errors, and for any proposed set of code words, {B₁, . . . , B_{N}}, the error correction properties of this can be directly and exactly assessed by examining all possible corrupted versions of each Bᵢ and noting for which corruptions this process corrects them. Various embodiments of such methods are provided by specific Hamming Codes, which are strings sets {B₁, . . . B_{N}} that have optimal or highly effective and uniform error correction by this means of correcting to the Hamming distance closest allowed code. In general, many other error correction encoding schemes are known to those skilled in the art of coding theory, and any of these schemes defined for K-bit strings can be used to provide K-mer code word sets that also have powerful error correction capabilities, and which can be used here to correct for possible decoding hybridization errors. In general, this provides a mechanism with arbitrarily good power to correct errors, at the cost of larger K, and therefore more physical decoding probes and more decoding reactions.

In various embodiments, decoding probes usable in these electrical or optical decoding methods may comprise short oligonucleotides, such as for example in the range of from about 8 to about 25 bases. Further, any two such targets have multiple mismatches between them, to reduce cross-hybridization, preferable 2 or more, and preferably 4 or more. In various embodiments, decoding probes may comprise PNA probes, so that a short probe can have stronger binding and higher Tₘ, and so that the impact of single mismatches can be greater on reducing cross-hybridization. In various embodiments, all of the decoding methods disclosed herein can be used with electronic detection of decoding probe hybridization provided by the sensor chip array, or using optically labeled decoding probes such as a dye label, quantum dot label, or gold nanoparticle label, or any other label detectable by microscopy and compatible with attachment to a single molecule DNA oligonucleotide and localization of probe binding by microscopic imaging.

In various other decoding methodologies, the objective and benefit is to have a decoding method in which the number of decoding targets added to each probe is a number J that can be specified as desired, so as to control the amount of hybridization target added to the probes for decoding purposes. This can be achieved using the compact form of the first family of methods discussed above. For these methods, the binary codes string {B₁, . . . , B_{N}} are defined as follows: for the set of numbers {1, . . . , L}, for some L, a subset S of this set is represented by the K-bit string (b₁)(b₂) . . . (b_{K}), where b; =1 if i is in the subset S, and bᵢ=0 if it is not. This is the sometimes called the indicator function for the subset. For example, the subset {2,4} would have indicator string 0101000 . . . 0. There are 2^{K} such strings, corresponding to the membership indicator strings of all 2^{K} subsets of S. In the setting, the set of all strings that have exactly J 1's in them is defined as the codes. The number of such strings is known in combinatorics as “L choose J”, and is N=L!/(J! (L−J)!), where “n!” denotes n factorial=n×n−1× . . . ×2×1. When this set of code strings is used in specified “compact” forms of the decoding methods above, there is the advantage that for the physical encoding, wherein a target is added for every 1 occurring in the encoding string Bᵢ, there are always exactly J such 1's, and so exactly J hybridization targets are added to encode each hybridization probe. This process therefore has the advantage of controlling the amount of target material added for decoding, to be J oligonucleotide targets. For any desired number of hybridization probes N to be encoded, and any desired J>1, L can be chosen large enough to that L!/(J! (L−J)!) in ≥N, and therefore provides enough such codes. The cost of achieving this as that L encoding probes are required. For example, suppose there were N=1024 hybridization probe types. One option would be to take all K=10-bit binary strings, and assigns all these as codes. However, in the above methods, each probe would get linked to either 10 targets (in the non-compact scheme), or a variable number of targets between 0 and 10 in the compact schemes. The decoding would require 20 reactions in the full scheme, or 10 in the compact scheme. However, restricted to linking to J=2 targets per probe, L=46 encoding probes and reactions are required, but allowing J=3 reduces this to L=20, and J=4 allows L=15. These are generally more desirable, such as required 15 probes and reactions, but only needing to add 4 decoding oligo targets to each probe. However, these do not provide any error correction capability, as a single bit error would produce a 3 element or 5 element subset indicator string, which does not have a unique Hamming distance closest string in 4 element set indicators.

Chip-Based Systems

In various embodiments, chip-based, multiplex hybridization probe sensor devices are deployed in a compact, low cost electronic instrument that is suitable for distributed use, field use, or point-of-care use. Such instrument architectures comprise a chip board that mates to the chip, motherboard that hosts the chip, and FPGA-based control and data transfer subsystem, a data processing subsystem, which may comprise CPUs, GPUs, FPGAs or other signal processing hardware, a fluidics subsystem, on instrument data storage, and of-instrument data transfer systems.

In various embodiments, a molecular electronic sensor chip device configured for genetic analysis may be deployed within a cartridge that also contains some or all necessary reagents to prep an input sample as required for “on-cartridge” analysis. In various embodiments, analysis of an input sample on-cartridge allows for a partial or fully dry instrument platform. In various embodiments, this cartridge is run on a desktop instrument that provides for a user interface, a control computer controlling chip and system functions, control of any on-board fluidics or actuators that control on-cartridge fluidics to supply a prepped input sample and necessary reagents to the chip, transfer data from the chip to internal storage or data processors, such as FPGA, GPU or CPU data processors, and transfer data off-instrument via direct internet or wireless connectivity to remote or cloud-based data centers. In this way, such genetic analysis systems provide a sample prep system, internally or as a companion instrument, capable of converting bio-samples of interest to a form suitable for on-chip application.

In various embodiments, a molecular electronic sensor system for genetic analysis can have a compact form factor suitable for mobile use. In other embodiments, such a system can have a highly compact form factor suitable for point-of-use or point-of-care, or suitable for field deployment. Such point-of-use applications may include testing stations deployed at airports, transportation hubs, hospitals, schools, stadiums, cruise ships, transport ships, or other major sites of congregation, or deployed at sites of business or commercial activity. In various embodiments, such testing stations can be configured for home use, e.g., for personal testing and health monitoring. In various embodiments, point-of-use systems may be deployed in the field for military, police, customs or border control point-of-contact testing, or other in-the-field testing and monitoring applications, such as testing of commercial vehicles, trains or aircraft for the presence of certain pathogens.

Methods and Applications for Infectious Disease

In various embodiments, testing or monitoring applications include testing for the presence of pathogens, such as, for example, testing for parasites, fungi, bacterial pathogens or viral pathogens. Such parasites include, but are not limited to, Malaria, Giardia, and Toxoplasmosis. Such bacterial pathogens include, but are not limited to, Salmonella and E. coli. Viral pathogens include, but are not limited to, influenza, flu viruses, cold viruses including rhinovirus, adenovirus, and human corona virus, HIV, Ebola, Dengue, Hanta, Zika and West Nile viruses, SARS, MERS, and SARS-CoV-2 virus (the etiological agent of COVID-19), and novel viruses of DNA or RNA type related to or unrelated to these having a known genetic sequence to define sequencing for the hybridization probes.

In various embodiments, a genetic test system is deployed at a testing site. A primary bio-sample is collected and delivered to the testing site where the sample is to be tested for the presence of a given pathogen or pathogen strain. At the testing site, a sample preparation process is applied to the primary bio-sample to produce a product suitable for application to the molecular electronic sensor array chip device that comprises a multiplicity of hybridization probe sensors configured to target a pathogen or pathogen strain of interest. After application of the prepped sample to the sensor array chip, device signals are readout, the signals undergo primary local signal processing, and then these data are transferred to a centralized or cloud-based server for subsequent analysis or testing outcome report generation.

In various embodiments, a testing site may comprise a centralized testing facility of high capacity, e.g., for a business, hospital or other organization, or for a region such as a city, county, state, or country. In other embodiments, the testing site can be a field deployment site, or a point-of-contact site, such as at an airport, transportation hub, or major gathering site such as an arena or stadium, or at an immigration control checkpoint or temporary monitoring point set up by the military, police, or government officials. In other embodiments, the testing site can be a mobile van deployed to sites as needed. In other embodiments, the testing site may be in a residential home occupied by private individuals. In other embodiments, the testing site can be autonomous environmental monitoring stations deployed into the field, stationary or mobile, including driving, flying or aquatic drones that monitor samples acquired locally from the environment, such as through filtering of air, or water, or trapping of known disease vectors or carriers in the environment, such as insects, rodents, bats or birds, or aquatic snails. In various embodiments, mosquitoes are one such vector.

In various embodiments, a primary bio-sample can be obtained as a swab of a surface that collects material deposited on the surface, as filtered material collected from air or water, or a water sample, or as a bodily fluid sample or buccal swab or saliva or excrement or tissue sample provided from a person or animal, or as a sample of a food item, or agricultural product.

In various embodiments, sample collection may be done in close proximity to a test system, such as within 1 foot, 10 feet, or 100 feet, and those samples rapidly delivered to the test system, such as within 10 seconds, one minute, 10 minutes or 1 hour, in order to have the benefit of distributed sample collection combined with rapid testing and test results. In various embodiments, the sample collection includes the assignment of a unique ID to the sample, such as an alpha-numeric code, serial number, barcode or QR code, to be used for sample tracking, and affiliation of final report back to the sample. In various embodiments, other identifying information may be collected and attached to the sample or affiliated with the sample ID, such as personal identifier, such as a personal name, social security number, government issued ID number, employee number, or date of birth, facial image or fingerprint.

In various embodiments, a sample preparation process comprises a PCR-based amplification method applied to the sample to produce amplified DNA material for detection. In other embodiments, the sample preparation process is a process to extract and purify DNA or RNA without any amplification to produced purified material for detection. In various embodiments, the sample prep process is performed in a separate instrument from the sensor chip instrument, and the prepped sample is then transferred to that instrument. In other embodiments, the sample prep process is performed on a subsystem integrated into the same instrument that runs the sensor chip device.

In various embodiments, a pathogen of interest is a pathogenic bacteria, such as E. coli, Salmonella, or Listeria, and the corresponding hybridization probes include specific DNA probes common to many strains of such bacteria of interest. In other embodiments, the pathogen is interest includes the specific strains of bacteria, and the corresponding hybridization probes include strain-specific DNA probes.

Application to Viral Pandemics and COVID-19 Pandemic

In various embodiments, a pathogen of interest is a virus, such as influenza, flu viruses, cold viruses-including rhinovirus, adenovirus, and human coronavirus, HIV, Ebola, SARS, MERS, and SARS-CoV-2 (the etiological agent of COVID-19), and novel viruses of DNA or RNA type related to or unrelated to these, such as those having a known genetic sequence that can define a sequence in a hybridization probe. In such cases, the hybridization probes include specific DNA probes common to many strains of viruses of interest. In other embodiments, the pathogen includes the specific strains of such viruses, and the corresponding hybridization probes include strain-specific DNA probes.

In various embodiments, primary data analysis on-system includes data reduction algorithms that reduce the amount of data needed to be transferred off-system. Such methods may include discarding uninformative portions of a signal trace, subsampling or parameterization of parts of a signal trace, and general data compression algorithms such as methods utilized in zip, gzip, bzip, and other common compression utilities. In various embodiments, the primary analysis also includes analysis of traces to produce a net hybridization intensity score for each probe on a sensor chip, and in various embodiments, a final call of detection, non-detection, or indeterminate measurement for each probe on the sensor chip. In other embodiments, such analysis is done in the off-instrument phase of an analysis. In other embodiments, the off-instrument analysis includes the generation of a final report that affiliates sample identifiers with the outcome of the test for the presence of pathogens of interest. Such identifiers may include a subject name or assigned ID or other identifier provided at the point of sample collection, as well as sample identifiers such as the time and place of sample collection, and time and place of sample processing on the sensor chip system.

In various embodiments, genetic analysis testing by a molecular electronic sensor system herein is performed rapidly, with the time from providing a primary bio-sample to completion of analysis and report generation being less than about 24 hours, and more preferably, less than about 8 hours, less than about 4 hours, less than about 1 hour, less than about 30 minutes, or less than about 15 minutes.

In various embodiments, a molecular electronics sensor system configured for genetic analysis is used in the monitoring of the pandemic disease COVID-19, a viral disease outbreak in 2019 originating in Wuhan, China. In accordance with the present disclosure, the hybridization probes for the individual sensors in an array are selected to be complements to segments of the genome of the underlying virus, the Severe Acute Respiratory Syndrome Coronavirus 2, also designated SARS-CoV-2. The SARS-CoV-2 virus has a single stranded RNA genome of 29,902 bases. One exemplar sequence for this genome is available at the Genbank® database as accession ID LC528232 (see https://www.ncbi.nlm.nih.gov/genbank/). Thus, in embodiments where a DNA hybridization probe is configured to directly detect the genomic material by hybridization, there will be DNA-RNA hybridization, and the sample prep must therefore be designed to extract and purify RNA from the primary bio-sample. In various embodiments where the sample prep comprises a PCR amplification of the genome, the process would comprise a reverse-transcriptase mediated PCR that produces amplified DNA product, either of specific target segments, or non-specific segments of the entire genome, and the resulting hybridization detection is DNA-DNA hybridization. By taxonomy, the pandemic virus is a specific strain of the Severe Acute Respiratory Syndrome-related Coronavirus (SARSr-CoV), which is a species of coronavirus that infects humans, bats and certain other mammals. There are hundreds of known strains of this virus, and hybridization probes must be chosen for sequence segments that distinguish the COVID-19 strain from other harmless coronavirus strains, or other disease causing strains, such as the strain designated SARS-CoV, which caused SARS disease outbreak in 2002 in Guangdong Province, China. There are numerous sequence differences between these strains, providing many candidates for distinguishing target sequences.

In various embodiments for COVID-19 testing, primary samples comprise environmental surface swabs or air filters, and such testing provides for monitoring of the presence of the virus in a target location where such samples are collected. In other embodiments for COVID-19 testing, primary samples comprise bio-samples such as saliva, buccal swab or mucus samples from individuals, and such testing provides for detection or diagnosis of subjects with active viral infections. In embodiments of such testing, the present sensor array device provides for rapid, distributed testing. In various embodiments, the system provides for a test in less than about 1 hour, or less than about 30 minutes, or less than about 15 minutes.

Other Infectious Disease Applications

In various embodiments, sensor systems according to the present disclosure can be applied to testing for, and response to, outbreaks of influenza, such as Influenza A and Influenza B strains. In these instances, hybridization probes are designed to have an oligonucleotide sequence that is complementary to a segment of an Influenza A or an Influenza B genome. In various embodiments, sensor systems can be used in the testing for, and the response to, various bacterial diseases, such as when the food supply is contaminated by E. coli or Salmonella. For example, this could be an outbreak where lettuce is contaminated by E. coli, or where ground beef is contaminated by Salmonella. In various embodiments, testing platforms are deployed in point-of-use format to sites of food production, such as farms, fields, and processing plants. Testing platforms may also be deployed in point-of-use form, as well as mobile or distributed permanent or temporary monitoring installations, to points of distribution, such as warehouses, shipping centers, or grocery stores and restaurants. In various embodiments, testing platforms are deployed to the end point of consumption, such as in the home, for home-based testing. In various embodiments, aggregated cloud-based analysis, including Big Data, A.I. and machine learning techniques, can be used to track the outbreak and pinpoint the source.

In various embodiments, sensor systems according to the present disclosure can be used in testing for sexually transmitted diseases (STDs). In accordance with the present disclosure, it is an advantage that such molecular electronic hybridization sensor systems can be deployed for rapid, low-cost testing in highly distributed fashion, such as in community or field clinics, or for use in the privacy of a residential home. For STDs, the causal pathogens to be detected may be parasites, such as Trichomoniasis, or a fungi, such as Candidiasis (yeast infection), or bacteria such as Syphilis, Gonorrhea, or Chlamydia, or viruses such as Herpes, HPV, EBV, Hepatitis, and HIV. In such applications, the primary samples required are clinically well established, and may be a blood sample, or a swab of bodily fluids or of discharges, or from open sores. In various embodiments of such testing, the present sensor array device provides for rapid, distributed testing. In various embodiments, the sensor system provides for a test in less than about 1 hour, or less than about 30 minutes, or less than about 15 minutes. In various embodiments, the sensor system provides the advantage of extreme personal privacy, with systems and test kits that can be used entirely within the home.

Experimental Molecular Electronics Sensors and Chips and Illustrated Embodiments

Various embodiments of a molecular electronics sensor configured for genetic analysis in accordance with the present disclosure may be further understood and appreciated with reference to the drawing figures, as per the discussions below:

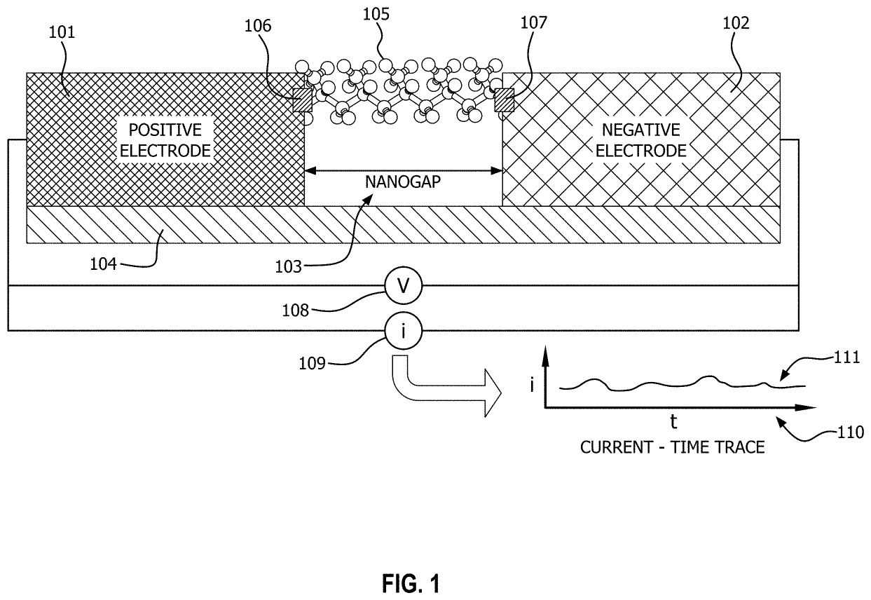

With reference now to the drawing figures, FIG. 1 illustrates a general embodiment of a molecular electronics sensor circuit. The circuit comprises a first nanoelectrode 10 (e.g., positive, or source) and a second nanoelectrode 102 (e.g., negative, or drain) spaced apart from the first nanoelectrode 101 by a nanogap 103. In this circuit, a voltage “v” 108 is applied to the circuit and a measurable electrical parameter, in this case current (i) 109, is measured in the circuit over time. In other configurations, voltage is applied and another parameter measured. In various embodiments, the measurable electrical parameter is something other than current, such as voltage or impedance. The molecule 105 shown between the electrodes is an electrically conducting bridge molecule 105 that closes the circuit between the positive 101 and the negative 102 electrodes. The scale of the nanogap 103 between the electrodes in any pair of electrodes 101/102 is fixed based on the length of the bridge molecule 105 or bridging molecular complex of interest, and may be about 10 nm. In various embodiments, the nanogap 103 may measure from about 3 to about 10 nm, from about 10 to about 30 nm, from about 30 to about 100 nm, from about 100 to about 1000 nm, or from about 1000 to about 10,000 nm.

With voltage “v” 108 thus applied, conformational changes in the bridge molecule, or external influences, produce perturbations 111 in the measured electrical parameter, i.e., current over time, as shown in the inset trace of current versus time 110 directly below the sensor drawing. That is, molecular interactions, or “events,” are transduced into signals 111 or detectible signatures present in the measured current versus time trace 110. Molecular interactions between the bridging molecule 105 and various analyte targets change the conductivity of the bridge molecule, resulting in perturbations 111 in the measured current over time 110 corresponding to the molecular interactions between the bridge molecule and various analyte targets. The resulting system has the potential to be used as a sensor for a great variety of molecular interaction processes. As detailed in various embodiments herein, the bridge molecule may be modified into a molecular complex by conjugating at least one probe molecule to the bridge molecule, such as an oligonucleotide hybridization probe. The process of forming a molecular bridge complex may be before or after the bridge molecule 105 is bonded between the two electrodes 101/102 in the pair of electrodes. In various embodiments, a DNA or RNA hybridization probe is conjugated to the bridge molecule by its 3′ or 5′ end, and it is the hybridization probe, not the bridge molecule per se, that interacts with various target molecules to produce the perturbations seen in the measured electrical parameter, such as current in the circuit over time.

In FIG. 1, and in accordance with various aspects of the present disclosure, the positive 101 and negative 102 electrodes are disposed on an insulative support layer 104 otherwise referred to as a “substrate.” The substrate 104 may comprise Si or SiO₂ on Si or Al₂O₃ on Si. In various embodiments, the insulative support layer 104 under the electrodes 101/102 may comprise a silicon-on-insulator wafer or a physical slice therefrom. Throughout the present disclosure, the positive and negative electrodes 101/102 shown in FIG. 1 may also be referred to as first and second nanoelectrodes or source and drain electrodes. In any case, this pair of electrodes comprise first 101 and second 102 nanoelectrodes spaced-apart by a nanogap 103 as illustrated. The bridge molecule 105, e.g., a biomolecule, molecular wire or nanoribbon, is bonded at each of its ends 106/107 to the first and second electrodes 101/102 so as to electrically connect the two electrodes and close a circuit, but in a manner so as to stay distanced and physically separate from the underlying insulator support layer 104 and to be configured as a true bridging structure suspended over the nanogap 103 that lies between the electrodes in a pair of electrodes.

In the basic sensor circuit shown in FIG. 1, the first 101 and second 102 electrodes may comprise source and drain electrodes. Further, there may be a gate electrode (not illustrated) capacitively connected to the positive and negative electrodes. In various embodiments, a gate electrode, when present in the sensor circuit, may be positioned within or underneath the insulator support layer and directly beneath the gap between the positive and negative electrodes.

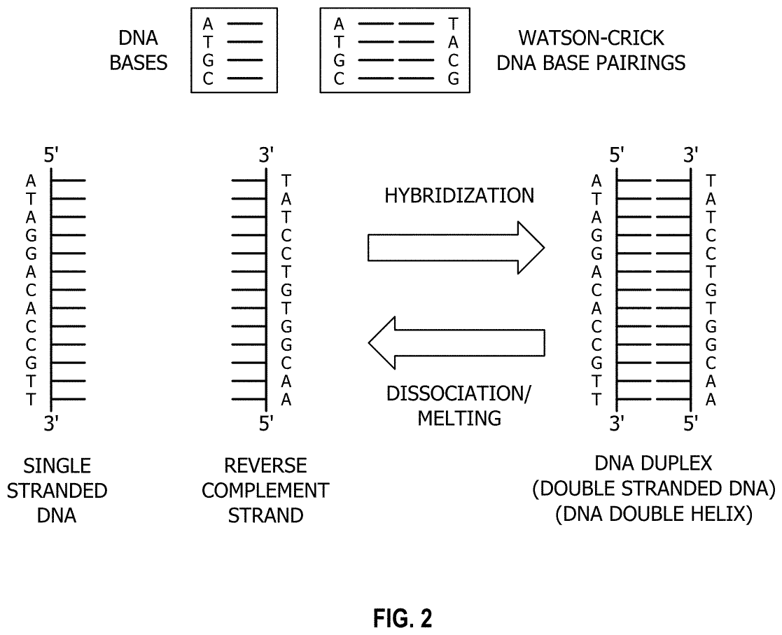

FIG. 2 sets forth aspects of oligonucleotide hybridization that is relevant to the functioning of the molecular sensors disclosed herein. In general, genetic analysis, including measuring the presence of, or a concentration of, DNA or RNA segments of interest in a sample, has previously comprised molecular biology tools such as PCR, DNA microarrays, and DNA sequencing. Older techniques predating these, such as Southern Blots (for measuring DNA targets) and Northern Blots (for measuring RNA targets) have also been applied to these problems. All such assays, including Southern and Northern Blots, employ the process of DNA “hybridization” as a fundamental part of the detection scheme.

As shown in FIG. 2, hybridization is a chemical reaction wherein a single stranded DNA molecule will, with high efficiency, bind to its specific reverse complementary strand in solution as shown. The DNA bases pair in accordance to Watson-Crick pairing such that A binds to T, T binds to A, G binds to C, and C binds to G. As shown, the result of hybridization is a DNA duplex, which is a double stranded DNA double helix. Also as shown, hybridization is reversible. The reverse reaction is referred to as dissociation, or melting, and is sometimes categorized by a melting temperature, denoted Tₘ. For simplicity, the DNA duplex is shown as a strip with pegs for the bases, absent the hydrogen bonding and other chemical details.

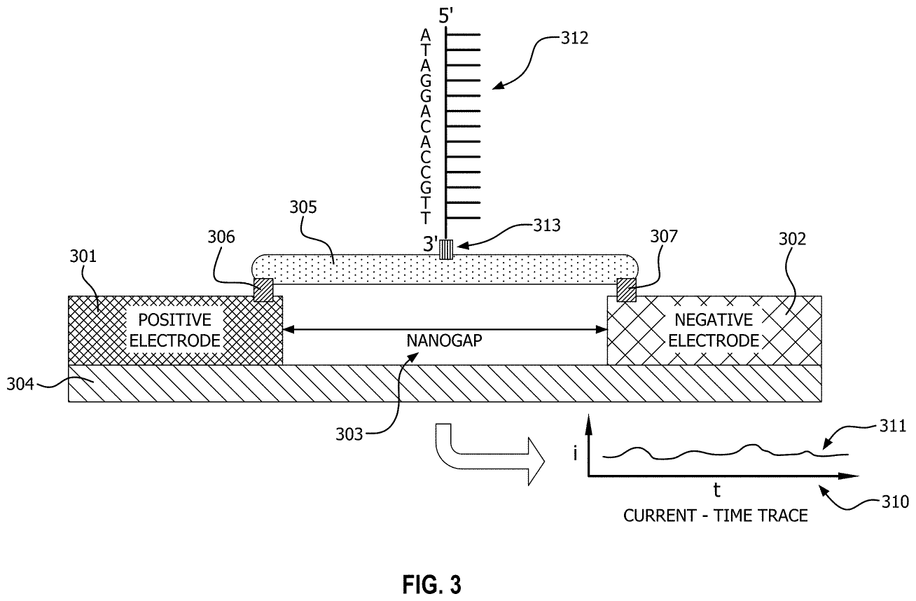

FIG. 3 sets forth a structure of a basic sensor device in accordance with various embodiments of the present disclosure, in which a hybridization probe 312, here a single stranded DNA oligonucleotide, is conjugated at its 3′ end 313 to a bridge molecule 305 that electrically connects the positive 301 and negative 302 electrodes. The positive 301 and negative 302 electrodes are disposed on a substrate 304. As detailed herein, the hybridization probe 312 may be an RNA segment, and in either case of DNA or RNA, may be conjugated to the bridge molecule by its 5′ end rather than its 3′ end, or at a position somewhere between the 3′ and 5′ ends, or in two positions along the segment such that the hybridization probe 312 is configured as a loop on the bridge molecule 305. Further, the hybridization probe 312 may comprise multiple linear oligonucleotide segments attached together by linkers. In general, a hybridization probe herein need only be a molecular construct or complex comprising a single stranded oligonucleotide segment having a hybridization probe sequence of interest. In various embodiments, the conjugation 313 between an end of the hybridization probe 312 and the bridge molecule 305 may comprise covalent bonding, such as afforded by a linker or click-chemistry coupling techniques.

In various embodiments, the bridge molecule 305 may comprise any molecule that can provide an electrically conducting connection between the first 301 and second 302 electrodes in a pair of nanoelectrodes. Such a conducting bridge molecule 305 could, in various embodiments, comprise a double stranded DNA segment, an alpha-helical protein, a polypeptide, a carbon nanotube, a graphene nanoribbon, a multi-chain protein such as a bacterial pilin filament or bacterial nanowire, or a conducting polymer such as PEDOT. In various embodiments, a bridge molecule 305 herein is fabricated by a “bottom up” chemical synthesis, and is made to have a defined chemical structure including engineered-in specific binding groups at precise locations in its chemical structure to provide the conjugation or binding sites for the hybridization probe 312 and for conjugation to each of the nanoelectrodes 301/302. In various embodiments, such a bridge molecule 305 has a specific conjugation site 313 to which the DNA or RNA hybridization probe is conjugated to, either covalently or non-covalently, through any of many possible conjugation mechanisms known to those skilled in the art of bioconjugation. In various embodiments, the conjugation 313 between hybridization probe 312 and bridge molecule 305 may comprise a click chemistry coupling, such as dibenzocyclooctyne-azide (DBCO-azide) or trans-cyclooctene-azide (TCO-azide), or other non-copper or copper click reactions, an 3-arylpropiolonitrile-thiol (APN-thiol) coupling, an amine-N-hydroxysuccinimide (amine-NHS) ester coupling, a biotin-avidin coupling, a peptide-tag based coupling such as Spy-Tag/Spy-Catcher, or an AviTag™ (GeneCopoeia, Inc.) or an aldehyde tag.

In various embodiments of the sensor illustrated in FIG. 3, the electrodes 301/302 provide exposed metal contact targets for binding the bridge molecule 305 or molecular complex 305/312. In various embodiments, the bridge molecule 305 may be conjugated at its ends to the electrodes 301/302 by a thiol-metal bond, dithiol-metal bond, amine-metal bond, carbene-metal bond, or diazonium-metal bond, or other carbon-metal bonds, or the conjugation 306/307 may comprise a metal-to-metal binding peptide association. Metal binding peptides (or, referred to more generally as “material binding domains”) may comprise a gold binding peptide (GBP) having the amino acid sequence MHGKTQATSGTIQS (SEQ ID NO: 1). At one or both ends of a bridge molecule 305 herein, this metal binding peptide may be repeated in tandem about 2 to about 6 times, separated by short GS rich linkers, so as to ensure a more robust bond between the metal electrode and the peptide. Alternatively, a palladium binding peptide may be incorporated at the ends of the bridge molecule, having the amino acid sequence QQSWPIS (SEQ ID NO: 2). The conjugation 306/307 between metal electrode and an end of a bridge molecule may also be achieved by applying a bifunction linker with any of these or other binding groups to attach one end of the bridge molecule to the electrode, with a short linker such as a PEG group or other carbon chain about 1 to about 3 nm long, presenting an arbitrary second conjugation group as the head group, such as a click chemistry group, which can then be used to conjugate to a cognate group on the bridge molecule.

In FIG. 3, the DNA hybridization probe 312 is depicted to illustrate that the logical structure is a sequence of bases, with the important backbone orientations (3′ and 5′ ends) indicated, and to show the logical Watson-Crick base pairings (A-T, G-C in DNA, as well as U-T in RNA-RNA or RNA-DNA pairings, neither shown), but the illustration does not indicate the double-helix physical structure of the duplex, or the chemical hydrogen bonds involved in base pairing for the sake of simplicity. The reverse process, where a duplex molecule separates into independent single strands, is referred to as dissociation or melting, and it is a strongly temperature dependent process, the transition temperature being known as the melting temperature (Tₘ), which depends on the solution composition as well, and to a lesser extent on the strand concentration, and which can also depend on other chemical, steric or entropy effects like the proximity of other molecules or surfaces, or the tethering of DNA to other molecules. For DNA freely present in solution at substantially below the melting temperature, the duplex form is stable and long lived. However, in solution at substantially above the melting point, the duplex is unstable and correspondingly short lived. Within a few degrees C. of the melting point, the “on” reaction (formation of a duplex) and “off” reaction (separation of the duplex into probe and target strands) are both commonplace. Generally, hybridization is a chemical reaction obeying Boltzmann distribution kinetics, and as such, the reaction is reversible. In various aspects, the precise definition of the melting point is the temperature where strands are equally likely to be bound and unbound for the given solution conditions. In common physiological solution conditions, melting points for duplexes shorter than about 10 bases may be below room temperature (25° C.), while for duplexes about 50 bases or longer, or those with higher G-C pair content, Tₘ may approach the boiling point of water (100° C.). In typical buffer solutions used in molecular biology, the salt levels and divalent cation levels (such as Mg++) have a moderate effect on the melting point, and therefore an effect on the kinetics of hybridization and dissociation. At low concentration of the complimentary strands, it may take a long time for duplex formation to occur, as the strands must first meet, primarily by diffusive transport.

Similar to DNA, any combination of complementary DNA and RNA strands can pair by hybridization in this way, as can strands of DNA or RNA that contain various nucleotide analogs such as locked nucleic acid (LNA) or peptide nucleic acid (PNA), or various other chemically modified nucleotides. Longer segments of DNA that match along a segment will form a duplex pair along that segment, even if other portions of the longer segment, such as the ends, do not match, although the presence of single stranded free ends may result in a slightly lower melting point. Strands that are not perfectly matched can bind, but the presence of mis-paired bases substantially lowers the melting point, and these partial duplex structures may not be stable under many conditions of interest. This relatively lower Tₘ for mis-paired strands (sometimes called cross-hybridization) can be used advantageously to design hybridization detection assays wherein an exact match is preserved over a finite period of time and produces an enduring detectable signal, while all the many possible mismatch interactions with off-target fragments can be eliminated from producing signals at all, or that otherwise result in signals that are substantially below some form of detection threshold.

With continued reference to the basic sensor device of FIG. 3, the length of the sequence of the hybridization probe 312 intended for hybridization to a target DNA or RNA molecule may be from about 4 to about 200 bases, or from about 10 to about 100 bases, or from about 12 to about 50 bases. In various embodiments, the length of the sequence of the portion of the hybridization probe 312 intended for hybridization may be from about 10 to about 15 bases, or about 13 bases. In applications requiring single base discrimination, such as SNP genotyping or pathogen strain determination, the probe length is preferably from about 10 to about 35 bases in length, and more preferably from about 15 to about 25 bases in length. The probe 312 may also comprise other nucleic acids or nucleic acid analogs, such as RNA, PNA or LNA, which may provide stronger binding or greater specificity of binding. Such probes can have reduced length. The DNA hybridization probe may also comprise a fluorescent group, such as a 6-carboxyfluorescein (FAM) dye molecule on the 5′ end. Hybridization probes may also comprise groups for quality control or characterization in the synthesis and purification of the probe-bridge conjugates, or the characterization of the assembled sensors, such as in optically assessing whether a nanoelectrode has actually received and conjugated to a probe-bridge complex.

With continued reference to the basic sensor device of FIG. 3, the magnitude of the applied differential voltage across the electrodes in any pair of electrodes may be in the range of from about 0.01 V to about 10 V, from about 0.05 V to about 3 V, or from about 0.1 V to about 1.2 V. In various embodiments, the applied v may be a DC voltage, or an AC or time varying voltage, either sinusoidal, periodic or varying according to some other applied waveform. In various embodiments, there may also be a reference and working electrode provided to the solution phase that surrounds the sensor device, to set and control the potential of the solution relative to the potential of either electrode or to the ground of the circuit. Such electrodes in the surrounding solution may comprise platinum electrodes (or pseudo-electrodes as these are sometimes called), silver/silver chloride electrodes, or other types of electrodes used to set solution potential in an electrochemistry environment. The resulting current measured in various embodiments over time (e.g., the current-time trace 310 illustrated), may have baseline or typical magnitudes 311 in the range of from about 1 pico-Amp (pA) to about 10 pA, or from about 10 pA to about 100 pA, or from about 100 pA to about 1000 pA (1 nano-Amp (nA)), or from about 1 nA to about 10 nA, or from about 10 nA to about 100 nA.

In various embodiments of the basic sensor device illustrated in FIG. 3, there may also be a gate electrode, such as a gate electrode configured as a buried “back gate” electrode some distance underneath the insulating substrate, which may be used to apply an additional modulating voltage or field to the vicinity of the bridge and hybridization probe. It should be understood that any of the illustrated sensors may include a gate electrode. In various embodiments, such an electrode may be buried at from about 1 nm to about 10 nm below the surface, from about 10 nm to about 100 nm below, or from about 100 nm to about 1000 nm below, and may be approximately centered underneath the nanogap 303. In various embodiments, such a buried gate electrode may also have lateral localization such that it is localized at or near the footprint of the bridge, in one or both lateral dimensions, such as extending no more than about 10 nm, or no more than 50 nm, or no more than about 500 nm, or no more than about 1000 nm beyond the footprint of the bridge, in one or both lateral dimensions.

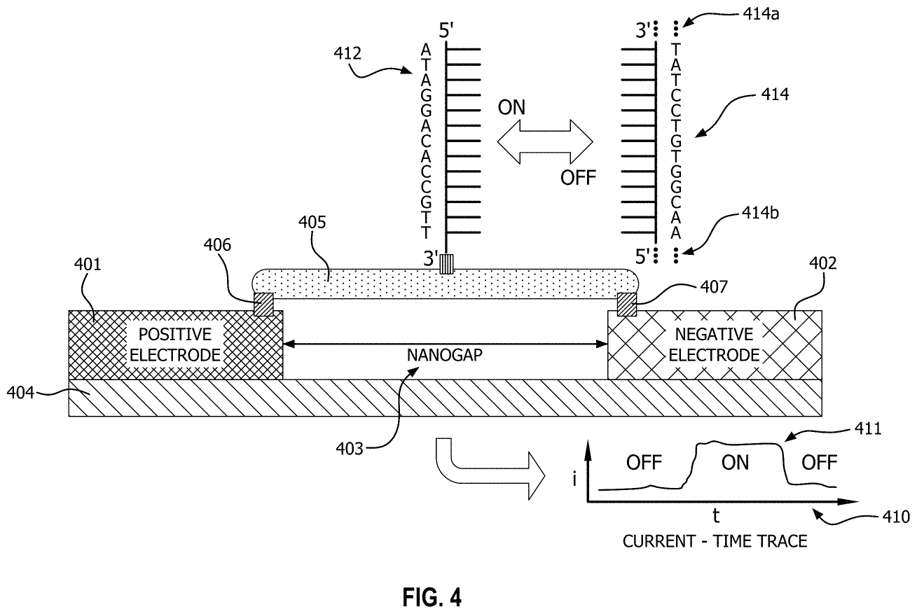

FIG. 4 illustrates a functioning sensor comprising positive 401 and negative 402 electrodes separated by a nanogap 403, a bridge molecule 405 connecting the two electrodes 401/402 and suspended over the nanogap 403, and a hybridization probe 412 conjugated at its 3′ end 413 to the bridge molecule 405. The sensor is illustrated with the probe 412 encountering a segment of a longer DNA molecule 414 (as indicated by the • • • symbols 414a/414b representing the target oligonucleotide length expands beyond the sequence illustrated) that is an exact match (i.e. reverse complement sequence), and the current over time trace 410 (shown below the sensor) changing, i.e., a jump in current up to a larger value 411, when the probe 412 has hybridized to this target 414. The hybridization, that is, the formation of the DNA duplex, is indicated as the “on” state 411 in the current-time trace 410. As shown, the measured current reverts back when the probe dissociates from the target to the “off” state again, the “off” state being the dissociated form between the hybridization probe 412 and the exact match target 414, i.e., the separate single stranded molecules. This signal 411 thereby provides a primary detection event, i.e., confirmation of the target being present in the sample provided to the sensor. If the exact match target is not encountered, there is no such distinguishable signal 411.

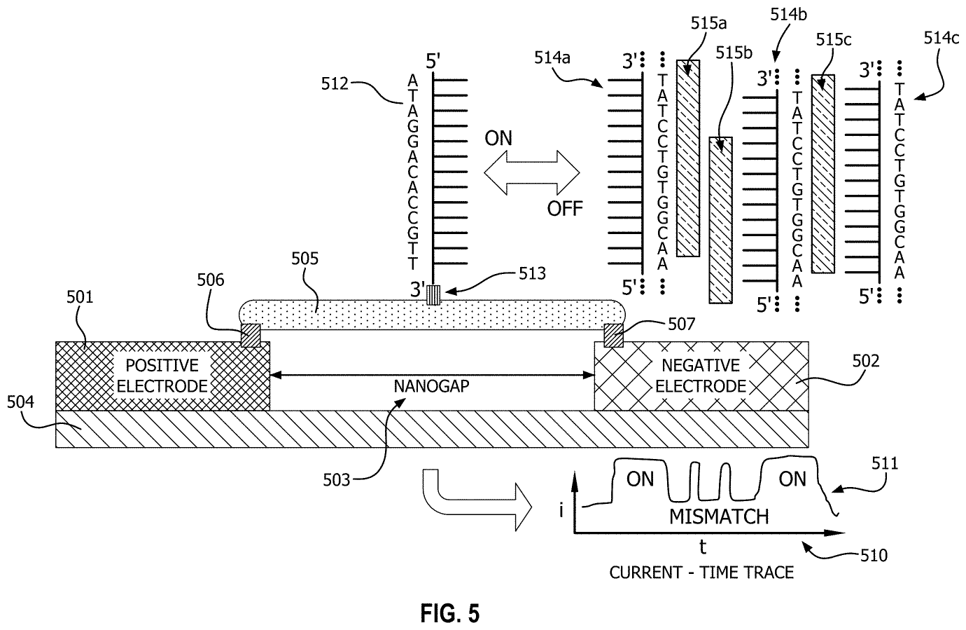

FIG. 5 illustrates a functioning molecular electronics sensor configured for genetic analysis interacting with a complex pool of six oligonucleotides (514a, b, and c, and 515a, b, and c) in solution and the effects seen in a measurable electrical parameter 511, such as current in a current-time trace 510, depending on what interactions occur with the hybridization probe. As mentioned, a sensor structure configured as per the present disclosure can function as a molecular electronics sensor for hybridization events. When such a molecular electronic sensor is exposed to a solution that contains single stranded DNA material, and if the hybridization probe encounters and binds by hybridization to its intended exact match target, a distinguishable signal is produced in the measured current.

Furthermore, as indicated in FIG. 5, when this sensor is exposed to a complex pool of DNA fragments, including not only exact match hybridization targets 514a, 514b and 514c, as indicated by the three specific complementary sequences shown, but also containing many non-matching or off-target fragments as indicated by the shaded “anonymous” oligonucleotide segments 515a, 515b and 515c, the hybridization events with exact matches produce the long duration detectible signals 511 (as per FIG. 4), whereas the many interactions with non-matching segments may result in no detectable jump in current. Many interaction events result in transient, rapid, or shorter duration pulses that are readily discriminated from the stronger (i.e., longer duration) exact match detection pulse in the current-time trace 510. This difference in signal characteristics between an exact match hybridization event and diverse non-specific interactions with other fragments, including interactions with just one base or a few base mismatches, provides for the primary detection of targets of interest in a sample, relative to a background of many other off-target fragments, even if such fragments differ by only a single base from the target oligonucleotide. Configured in this way, a molecular electronics single molecule hybridization sensor can detect the presence or absence of a target DNA of interest in a potentially complex pool of DNA, such as might be extracted from a bio-sample or environmental sample of interest by common sample preparation methods for extracting, purifying, or amplifying DNA (or RNA).

As shown in the current-time trace 510 in FIG. 5, the target “on” events result in a long step-up pulse, while the off-target events result in short step-ups of similar current magnitudes but of shorter duration. These details, particularly the amplitude and duration of current signals in FIG. 5 are not meant to be limiting, and in various embodiments, the exact target binding events might result in a step-up or step-down in magnitude of current, and any current spike might not return fully to a baseline in conditions when the target stays bound. The off-target events in various embodiments may also result in current pulses of shorter duration and/or smaller amplitude, or that may otherwise have a signature in the current trace that differs from the exact match signatures. In various embodiments, operating conditions, such as operating temperature, buffer chemical composition, pH, applied voltage, back gate voltage (if any), solution potential, and other parameters of the system, may be modified to enhance or optimize the ability to discriminate between exact match hybridization signals and various background interaction signals that the probe may engage in, or that result from noise arising from other aspects of the molecular electronic system. In terms of buffer composition, there are many factors that may be considered and that are known to those skilled in molecular biology that may impact hybridization reactions, such as concentrations of salts (such as NaCl, KCl), divalent cations (such as Mg++, Mn++, Ca++, Sr++), surfactants and solvents (such as Triton, DMSO, or a betaine), and the primary buffering agent, such as Tris, HEPES or MOPS.

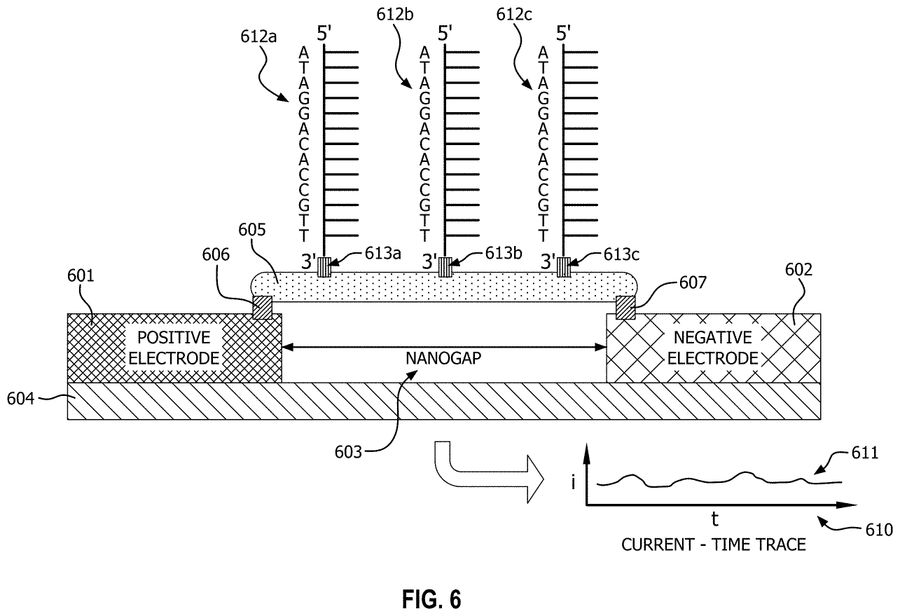

FIG. 6 illustrates a sensor structure comprising one bridge molecule 605 but more than one hybridization probe 612a, 612b and 612c conjugated to the bridge molecule. In this structure, a positive electrode 601 is disposed on a substrate 604 and spaced-apart by a nanogap 603 from a negative electrode 602 on the same substrate. The bridge molecule 605 has a first end conjugated to the positive electrode 601 by conjugation 606 and a second end conjugated to the negative electrode 602 by conjugation 607. The two or more hybridization probes 612a, 612b and 612c may be conjugated to specific and distinct sites on the bridge molecule 605, denoted 613a, 613b and 613c, respectively. In various embodiment, such multiple probes 612a, 612b and 612c may comprise identical DNA probes, or distinct DNA probes, that is, the hybridization probes may comprise a sequence to target the same oligonucleotide sequence, or different sequences. The benefits of incorporating multiple probes per sensor include increased total signal, increased signal-to-noise ratio, robustness against failures of probe conjugation (ensuring at least one probe is actually present), robustness against probes ending up in physically inaccessible configurations, and the ability to multiplex one sensor so as to detect multiple targets, for greater robustness, or breadth of detection. In various embodiments, the number of probes on a single sensor bridge molecule may number from about 2 to about 10, from about 10 to about 100, or from about 100 to about 1000 hybridization probes. Such probes may be located so as to point in similar directional orientations relative to the bridge, as suggested in FIG. 6, or they may be located so as to point in different directional orientations, including away from the bridge. A distribution of directionally oriented hybridization probes may provide a benefit of ensuring that at least some probes are spatially accessible once the bridge is in place on the electrodes. In various embodiments, spacing between such individual probes along the bridge molecule may be from about 1 to about 10 nm, or from about 10 nm to about 100 nm. In various embodiments, such a bridge molecule is fabricated by “bottoms up” synthesis in order to have these multiple probe conjugation sites at precisely defined locations in its chemical structure.

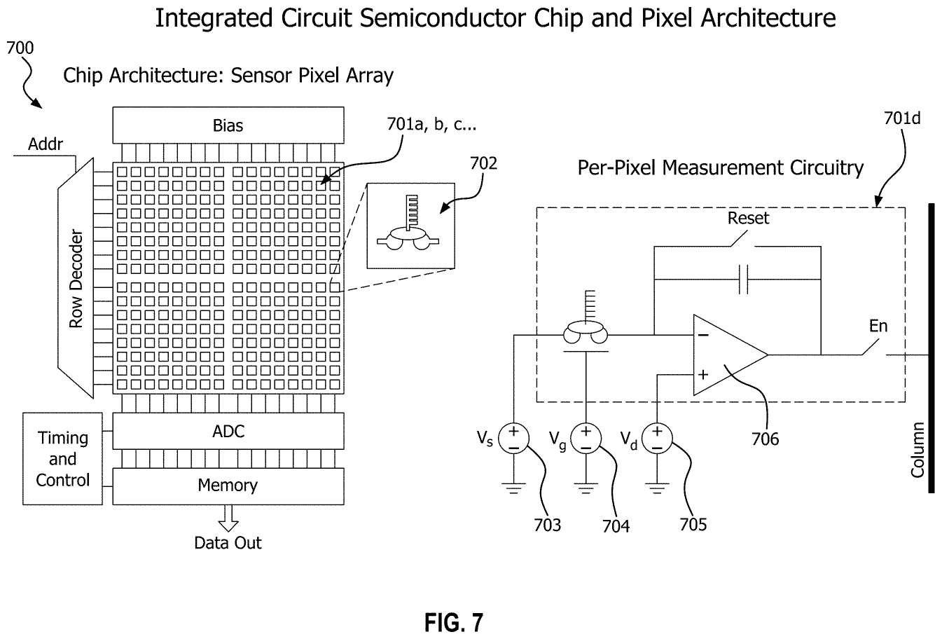

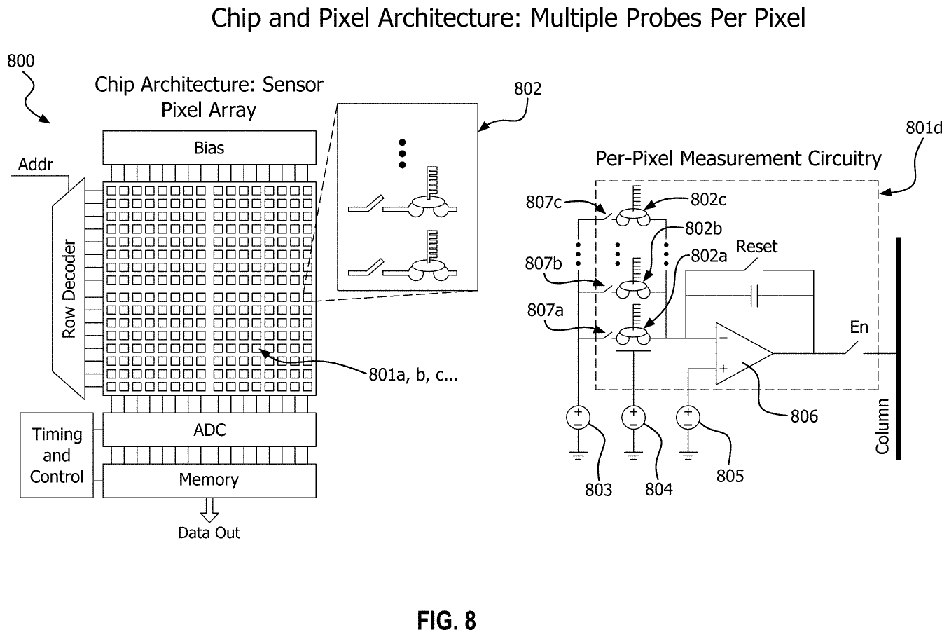

FIG. 7 and FIG. 8 illustrate chip and sensor pixel architectures, showing how individual sensors can be disposed in an array. In various embodiments, such sensor arrays can be configured for gene expression analysis of any cellular samples. In various embodiments, the analysis would comprise gene expression applied to tumor tissue, such as used in cancer diagnostics.

In various embodiments, such sensor arrays can be configured for SNP genotyping in human, animal, or other cellular samples. In various embodiments, this would be applied to SNP genotyping in humans.

With reference to FIG. 7 and FIG. 8, arrays of sensor pixels provide multiplexed hybridization probe detection and/or concentration measurement of targets in a complex pool. In various embodiments, each molecular electronics sensor is configured in its own sensor pixel. In other embodiments, multiple molecular electronics sensors are present in each individual sensor pixel. The level of multiplexing can be up to about 100 different probes, up to about 1000 probes, up to about 10,000 probes, up to about 100,000 probes, up to about 1 million probes, up to about 10 million probes, up to about 100 million probes, or even up to about 1 billion probes or more. A multiplexed sensor array provides an alternative to DNA microarrays for such high levels of multiplex detection, with the advantages of all-electronic, chip-based system, single molecule sensitivity, speed, low cost consumables and instrument, suitable for compact, mobile, portable, or point-of-use instruments.

In various embodiments, molecular electronics hybridization sensors are deployed on integrated circuit semiconductor chip devices, where such chips include the circuitry to supply voltages to the sensors, measure currents in the sensors, and transfer such data off-chip, and to control such operation.

In various embodiments, a multiplexed array of sensor pixels as per FIG. 7 (one molecular electronics sensor in each sensor pixel) may comprise multiply different hybridization probe sequences, i.e., different hybridization probes on different sensor pixels across the array of pixels. In other embodiments, an array of sensor pixels per FIG. 7 may comprise identical hybridization probes, i.e., the same hybridization probe present in each sensor pixel. A multiplexed array of sensor pixels per FIG. 7 may feature multiple hybridization probes per individual bridge molecule, as per FIG. 6, wherein probes may be the same or different on each bridge molecule, and same or different across the array of pixels.

In various embodiments, a multiplexed array of sensor pixels per FIG. 8 may comprise multiply different hybridization probe sequences, i.e., different hybridization probes on different sensor pixels and/or different hybridization probes per sensor pixel. In other embodiments, an array of sensor pixels per FIG. 8 may comprise identical hybridization probes, i.e., the same hybridization probes in each sensor pixel. A multiplexed array of sensor pixels per FIG. 8 may feature multiple hybridization probes per individual bridge molecule, as per FIG. 6.

FIG. 7 illustrates various embodiments of architecture of a chip 700 as an array of sensor pixels 701a, 701b, 701c . . . 701n, with such pixels providing the circuits needed to apply voltages and measure currents. In the various embodiments shown in FIG. 7, the chip 700 comprises a rectangular array of pixel circuits, each pixel comprising a single molecular electronics sensor capable of transducing molecular interaction events into electrical current signals, indicated schematically by the inset of the hybridization sensor 702, as well as having other major architectural blocks as labeled to apply the needed voltages (bias voltages), manage the transfer of measurements from the array, and their conversion to digital form, (e.g., Row Decoder and ADC, or Analog-to-Digital Convertor).

In various embodiments, the ADC converts each analog measured value to a binary digital value having a bit resolution of 8 bits, 10 bits, 12 bits, or 16 bits, or a bit depth selected in the range of about 1 to about 32 bits. In addition, other blocks as indicated in the architecture of the chip 700 in FIG. 7 include a local on-chip memory buffer (Memory), and the control circuitry (Timing and Control), which in various embodiments comprise circuits to produce, synchronize, and distribute clock signals, including PLL circuits. In various embodiments of pixel schematic circuit designs, a trans-impedance-amplifier (TIA) 706 circuit is included in any individual pixel 701d. This circuit schematic 701d shown allows application of Gate (V_{g}) 704, Source (Vₛ) 703, and Drain (V_{d}) 705 voltages to the sensor structure 702a, and the measurement of the resulting current by collection of charge onto a capacitor wired as illustrated, which may be reset to an uncharged state (or nominal state) by closing the indicated “Reset” switch and shorting the capacitor. Closing the “En” switch shown then outputs the measure data, i.e. the voltage across the capacitor, from the capacitor to the column bus and encoder.

With continued reference to FIG. 7, the circuit blocks indicated in the schematic, as well as the blocks of the pixel, or the pixel circuit itself, can be fulfilled by many possible detailed circuit designs and IC layouts, such as those in the art of VLSI Integrated Circuit Design, Digital Circuit Design, Mixed-Signal Circuit Design, and Analog Circuit Design. The architectures and schematics shown in FIG. 7 are not meant to be limiting on the use and configurations of such circuit designs or layouts that might provide similar functionality.

With continued reference to FIG. 7, and in various embodiments, the molecular electronic sensors can be deployed onto a CMOS chip device, which is a specific form of semiconductor chip and chip manufacturing process. The advantage of using CMOS chips is the very large manufacturing base for such chips, and related supply chains, as well as the aggressive scaling roadmap for such devices. The majority of chips presently made are of the CMOS type, including the common processors, memory, and digital imaging chips used in commercial products. Another advantage is that aggressive scaling has led to a shrinkage of the features on such chips down to near the 1 nm scale. Such manufacturing processes are in principle capable of producing nanoelectrodes needed for the present disclosed sensor, thereby enhancing the manufacturability of the devices disclosed herein.

In various embodiments, a chip 700, such as illustrated in FIG. 7, operates synchronously by each pixel 701a, 701b, 701c . . . 701n, first acquiring a single current measurement value, and then the array of such values transferred off-chip as a “frame” of digital data, in a row-by-row fashion, as indicated in FIG. 7, at a designated total frame rate, which in various embodiments may be up to about 10 frames per second, or up to about 100 frames per second, or up to about 1000 frames per second, or up to about 10,000, or up to about 100,000 frames per second. In various embodiments, the pixel array 701a, 701b, 701c . . . 701n may contain up to 100 pixels, 1000 pixels, 10,000 pixels, 100,000 pixels, 1,000,000 pixels, 100,000,000 pixels, or up to 1 billion or more of such pixels. The physical size of a chip 700 per FIG. 7 is related to the number of pixels, and in various embodiments may be as small as 1 mm² and as large as a full reticle size used in the photolithography, which may be up to approximately 30 mm².

With reference now to FIG. 8, each measurement pixel 801a, 801b, 801c . . . 801N, configured on the chip 800, may comprise more than one pair of nanoelectrodes and therefore more than one molecular electronics sensor per pixel, so that one measurement pixel circuit may be used to monitor signals from multiple sensor devices (each sensor device comprising a nanoelectrode pair with a bridge and hybridization probe molecule). In various embodiments, the measurement circuitry for applying voltages, measuring current, and reading out data in each pixel, such as 801d illustrated, is applied serially in time to each sensor 802a, 802b, 802c . . . 802_{M}, present within the pixel 801d, under control of transistor-based switches 807a, 807b, 807c . . . 807_{M}, in the sensor circuit capable of selecting any one of the sensors present for measurement. FIG. 8 illustrates these selection switches, labeled as “807a, 807b, 807c” switches in the pixel schematic 801d shown. By closing any single one of the available switches 807a, 807b, 807c, the measurement circuit in the pixel is electrically connected to just one sensor 802a, 802b, 802c, and thus connected to acquire data from only that particular sensor. Such data can then be transferred off the pixel in a frame transfer process, and then the pixel can be cycled on to the next molecular bridge circuit by opening and closing switches. In various embodiments, this is done synchronously, so that all pixels acquire data with their local switch (e.g., switch 1) closed, transfer a frame off the array, and then all pixels move on to closing then next switch (e.g., switch 807a), and so forth. By cycling through each of the switches in this way, frame by frame, and provided each pixel is configured to serve M sensors, a chip with N pixel circuits embodied on chip can acquire measurements from a larger (and potentially much larger) number, N×M, number of sensors. This strategy in general has the potential to lower chip costs, seeing that the price of chips is based on area, and the area required for the chip is dominated by the area needed for pixel circuits. Thus, this configuration has the benefit of greatly lowering the total pixel count and pixel circuit area required to serve the number of sensors of interest, versus having one pixel per sensor. In various embodiments, each pixel may comprise from about 2 to about 1024 sensors per pixel, and preferable in the range of about 4 to about 128 sensors per pixel. However, this form of multiplexing of sensors per pixel has the cost that measuring from all pixels takes longer, by the multiplier M, as it requires M times more frames, and also the disadvantage that each sensor is offline and not measuring while its switch is open, so that the respective current goes unobserved for at least part of the time, creating the potential to miss signal features in a current versus time trace.

In various embodiments, the chip pixel array architecture is such that nearby pixels in the array share a common staging area, where the many nanoelectrode pairs for these neighbors are all located, and suitably electrically routed back to these adjacent pixel circuits. This applies to both cases where each pixel has one sensor affiliated with it (e.g., FIG. 7), or cases of M multiple sensors present per pixel (e.g., FIG. 8). The use of such staging areas further improves the efficiency of circuit layouts, and allows the staging area to have a larger opening and better accessibility for nanofabrication or molecular assembly purposes, or to facilitate wetting by the solution applied to the chip.

In various embodiments of pixel arrays on chips such as depicted in FIGS. 7 and 8, multiple probes with the same target sequences are represented on all or only part of the array. In various embodiments, multiple measures of the same target can be aggregated or averaged together to produce a more robust detection of the presence or verification of the absence of the oligonucleotide target, or to provide more sensitive detection or lower detection limits, or to provide a measurement of the concentration of the target. For example, if N sensors have the same hybridization probe sequence to hybridize with the same target, and a fraction f of these probes register a detection event within a measurement time T of exposure to the liquid sample containing the target, then detection becomes more robust, sensitive, or accurate if a minimum threshold, fₘᵢₙ, is required for detection, f>fₘᵢₙ. Alternatively, the ratio of f/T, which is the rate of detection, provides a measure of the concentration of the target in the sample. In various other embodiments, more detailed analysis of the f(t) curve acquired during the time interval [0,T] provides various robust fits to the slope of this curve, or this curve could be fit to characteristic profiles or measured calibration curves produced by using known reference concentrations, to provide a measure of concentration from these multiple probe measurements on the chip array. Such aggregated measures also typically provide a related estimate of confidence or measurement uncertainty, such as a suitable mean and standard deviation. If such individual probes are otherwise directly providing concentration measures, for each probe, these can also be averaged together to produce a more accurate estimate of concentration, as well as providing error bars or confidence intervals on the measurement, based on the spreads or standard deviations observing in the set of individual measurements. This provides benefits of greater accuracy and measurement confidence for the concentration estimate of the target of interest.

In various embodiments of sensor arrays on a chip, e.g., per FIGS. 7 and 8, different hybridization probes sequenced for hybridizing different targets are represented on only some or all of the pixels of the array. In various embodiments, these multiple measures of different targets provide the ability for multiplex or in-parallel measurements of the set of targets of interest. This strategy has the advantage of lower cost of testing the targets, and faster testing of the targets, or simpler testing of the targets, or the use of less sample material or less reagents, to test for all the targets, versus separately testing for such targets on separate devices. This is generally referred to as multiplex testing or parallel testing, and is widely appreciated as a potential benefit to testing systems.

In various embodiments of sensor arrays on a chip, e.g., per FIGS. 7 and 8, both forms of multiple probes will be present, i.e., for the give set of hybridization probes with the respective targets of interest, each specific type of hybridization probe will be represented by multiple, replicated sensors on the array, thus providing the benefits of redundant, replicate measurement as detailed above, and the multiplex probes for the multiple targets will be represented on the array to also provide the benefits of multiplexing. The resulting compound benefit is multiplex testing, with confident measurements for each target that have the benefits of statistical replication for accuracy and confidence interval estimation. For this purpose, it is beneficial to allow for very large chip-based large arrays of probes, such as in various embodiments up to 100 probes, up to 1000 probes, up to up to 10,000 probes, up to 100,000 probes, up to 1 million probes, up to 10 million probes, up to 100 million probes, or up to 1 billion probes, or up to 10 billion probes.

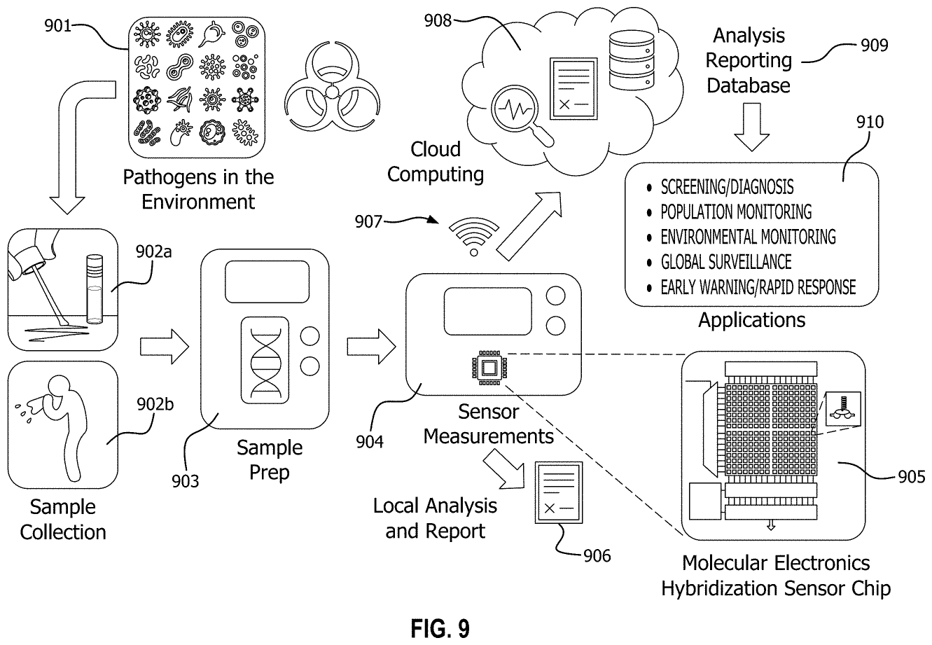

FIG. 9 illustrates embodiments of methods and systems for infectious disease pathogen detection in accordance with various aspects of the present disclosure. As illustrated, infectious disease pathogens exist in the environment 901, and include, for example, parasites, fungi, bacteria and viruses. As illustrated in the flowchart, a primary bio-sample is obtained, which in various embodiments may comprise material obtained from a human subject 902b, or a swab of an animal subject or an inanimate surface 902a, or other material collected from the environment, including plants or animals in the environment. As illustrated, the primary bio-sample is provided to a sample preparation system 903 that extracts and purifies the nucleic acid material contained within the primary sample, and puts it into a form suitable for application to a sensor chip device disclosed herein. This sample is provided to an instrument 904 that applies the sample to the chip 905 (e.g., a chip per FIG. 7 or FIG. 8), and controls the chip operations, and collects the sensor signal data from the chip 905. In various embodiments, the instrument 904 may locally analyze the data, record it locally 906, and produce a report on the pathogen content of the sample, such as on a screen/monitor on the instrument. In other various embodiments, this instrument transmits the data 907 to a remote cloud 908, where analysis 909, reporting out on the pathogen content of the sample, and databasing of results can occur. Such cloud-enabled embodiments, in conjunction with many deployed instruments, are well suited to large scale efforts, such as national, international, or global scale screening of samples for pathogens, such as for diagnosis of disease caused by pathogens, for monitoring the occurrence or spread of such disease across a population, or for monitoring such pathogens in the environment, and for global surveillance and early warning/rapid response efforts to contend with outbreaks of such infectious diseases (all indicated in 910).

In various embodiments, a primary bio-sample is acquired directly from a test subject or the environment, and then some form of sample prep is required to prepare materials to the proper state to apply to the sensor device for measurement. A primary sample for analysis may comprise tissue, saliva, mucous, buccal swab, blood, sweat, urine, stool, other bodily fluids, or exhaled air, or material filtered from air or water, or material swabbed from a surface. It could also be such samples acquired from plants or animals in the environment, or from food, or from known vectors in the environment that carry such pathogens, such as bats, rodents, mosquitoes or snails. In various embodiments, sample prep may result in a crude cell lysate extract containing DNA, or can comprise DNA further purified from the sample by standard purification column or filter paper purifications, or other extraction such as phenyl-chloroform. In various embodiments, the purified sample can be the result of applying any of the various forms of PCR amplification to the sample, which may be thermocycling or isothermal forms of PCR. In various embodiments, such sample prep is done by a self-contained sample prep device, or in other embodiments, a sample prep device may be integrated with the sensor platform, such as in the case of fully integrated point-of-use testing devices.

Experiments that have reduced these devices, methods and apparatus to practice are presented herein. Actual working sensors have been constructed and demonstrated to function.

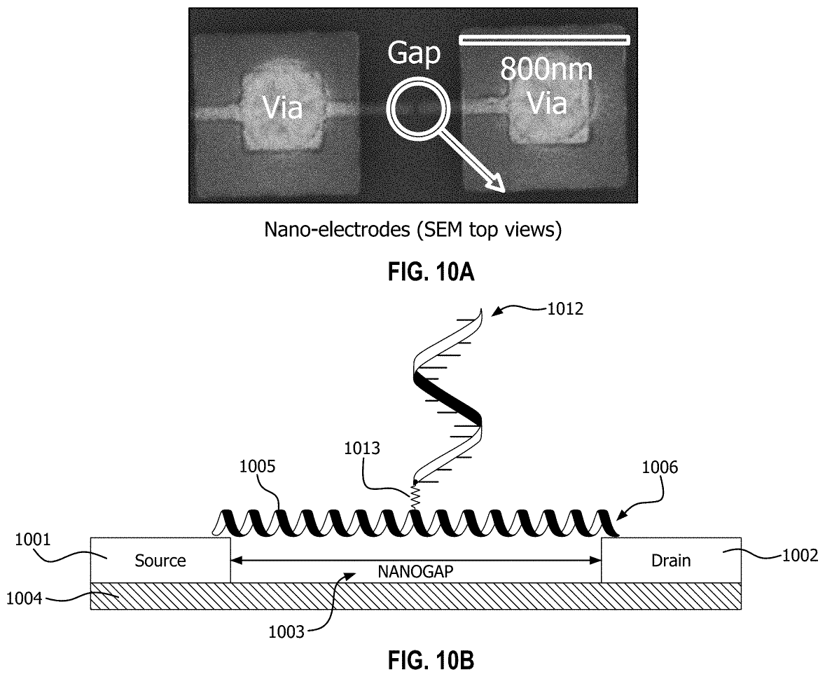

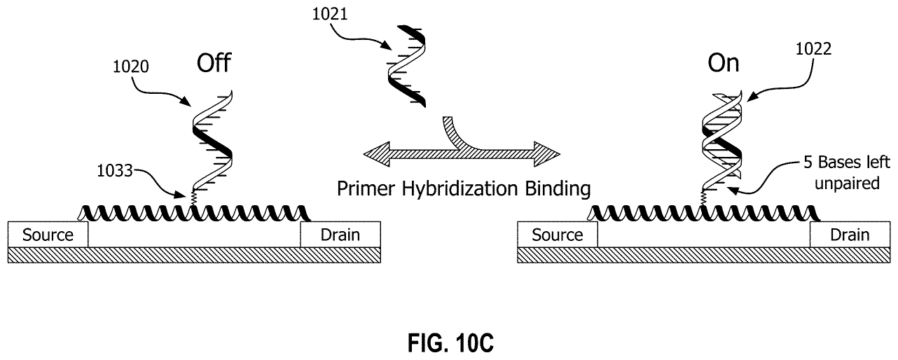

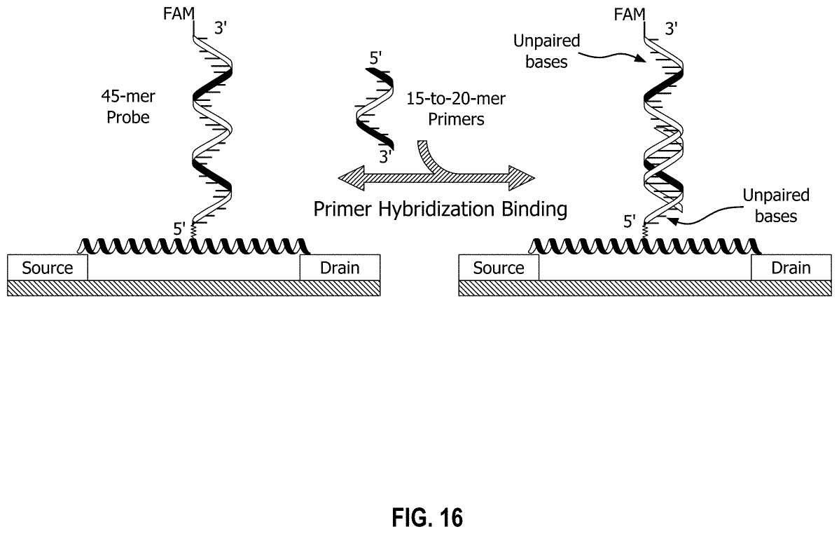

The sensor embodiments used for these experiments are shown in FIGS. 10A/10B and FIG. 16. As shown, these sensors consist of a DNA oligonucleotide probe conjugated to an alpha-helical peptide bridge molecule that is bound to a pair of ruthenium (Ru) nanoelectrodes, thus bridging the two. These devices are deployed on a 16 k pixel array CMOS chip device, with a 20 micron pixel pitch, fabricated in a 180 nm CMOS node, and which is a specific embodiment of the chip and pixel architecture shown in FIG. 7.

FIG. 10A shows an SEM of one individual molecular electronics sensor within an array of such devices fabricated on a chip using standard methods of e-beam lithography, sputtering deposition of the metal electrodes, and a lift-off process. As shown in the SEM inset, the nanoelectrode geometry comprises a nanogap between the two electrodes of about 15 nm to about 20 nm (labeled “Gap”), an electrode height of about 20 nm, and an electrode width (looking down onto the electrodes from above) of about 50 nm. As shown, the nanoelectrodes connect to exposed vias (800 nm) on the chip surface that in turn connect the sensor into the pixel amplifier circuit as in the schematic of FIG. 7. The chip produces 8-bit digital data, at a frame rate of 1000 frames per second. The chip is mounted in a flow cell capable of exposes a 10 μL solution volume to the sensor array. These devices are run on a custom desktop instrument platform, such as indicated by the sensor measurement instrument of FIG. 9, and data are collected on instrument, and transferred to a centralized private server, as well as the cloud for analysis and storage, by way of internet and broadband wireless connections.

As illustrated in FIG. 10A, each molecular electronics sensor in each sensor pixel comprises a source electrode 1001, a drain electrode 1002 spaced-apart from the source electrode 1001 by the nanogap 1003, and a bridge molecule 1005 conjugated to both the source and drain electrodes. The hybridization probe 1012 is conjugated to the bridge molecule 1005 by linker 1013. The bridge molecule 1005 comprises metal binding peptide portions 1006 at each end for conjugation to the metal electrodes.

The bridge molecule 1005 for these experiments is a 227 amino acid sequence polypeptide capable of forming an alpha-helical protein structure:

(SEQ ID NO: 3)
QQSWPISGSGQQSWPISGSGQQSWPISGSGAEAAAREAAAREAAAREAA
AREAAAREAAAREAAAREAAAREAAAREAAAREAAAREAAAREAAAREA
AAREAAAREAAAREACAREAAAREAAAREAAAREAAAREAAAREAAARE
AAAREAAAREAAAREAAAREAAAREAAAREAAAREAAAREAAAREAAAR
AGSGQQSWPISGSGQQSWPISGSGQQSWPIS

In various embodiments, a polypeptide having at least 80%, at least 85%, at least 90%, at least 95%, or at least 98% sequence identity to SEQ ID NO: 3 may be used as a bridge molecule herein.

The structure of this peptide comprises a repeat of the helix-promoting motif EAAAR (SEQ ID NO: 4), wherein a centrally located amino acid is replaced by a C to allow for cysteine-mediated conjugation between the polypeptide 1005 and the hybridization probe molecule 1012 as indicated in FIG. 10A. The two termini of this peptide consist of the repeats QQSWPISGSGQQSWPISGSGQQSWPISGSG (SEQ ID NO: 5), which comprise three repetitions of the metal binding peptide QQSWPIS (SEQ ID NO: 2), separated by short GSG spacers, which provides for binding of the peptide bridge molecule to the metal electrodes. In helical form, the length of this 227 amino acid peptide bridge molecule is approximately 25 nm. It can be used to bridge nanoelectrode gaps in the range of from about 15 to about 20 nm. This peptide was produced by bacterial protein expression of a synthetic gene encoding the peptide.

The conjugation 1013 of the hybridization probe 1012 to the bridge 1005 is achieved using a bifunctional cross-linker usable for thiol-to-azide linking, abbreviated APN-BCN, which is bicyclo[6.1.0]non-4-yn-9-ylmethyl (4-(cyanoethynyl)phenyl)carbamate. The conjugation product between APN-BCN and the peptide is purified using a desalting spin-column, and the product peptide reacted with a variety of hybridization probe oligonucleotides having an azide at the 5′ end, such that the 5′ end is conjugated proximal to the bridge through the APN-BCN linker. The various peptide/DNA complexes thus resulting are purified by size-exclusion chromatography with structures verified by SDS Gel electrophoresis.

In the experimental hybridization probe sensor of FIG. 10A, the hybridization probe 1012 conjugated to the peptide bridge molecule 1005 has the oligonucleotide sequence 5′-ACGTAGCAGGTGACAGGTT-3′ (SEQ ID NO: 6). The experimental target primer for testing with this particular hybridization probe was a 14-mer oligonucleotide having the sequence, 5′-AACCTGTCACCTGC-3′ (SEQ ID NO: 7). As illustrated FIG. 10B, when the probe 1020 and the primer 1021 are hybridized to form a DNA duplex 1022, a sequence of five (5) bases on the bridge end of the hybridization probe are left unpaired to the primer. The left structure in FIG. 10C having the single stranded hybridization probe 1020 represents the “off” state for the sensor, whereas the right structure in FIG. 10C, after primer hybridization to the duplex 1022, represents the “on” state for the experimental sensor.

In various embodiments, a fluorescein dye can be attached to a free hydroxyl group at the 3′ end of any of the hybridization probes in any of the sensor structures herein. The relative quantities of fluorescein for labeled sequences can be checked by UV-vis spectroscopy, for example.

The hybridization experiments herein were run at 25° C. and with the following buffer composition: 50 mM NaCl; 10 mM tris-HCl; 10 mM MgCl₂; 1 mM DTT, at pH=7.9.

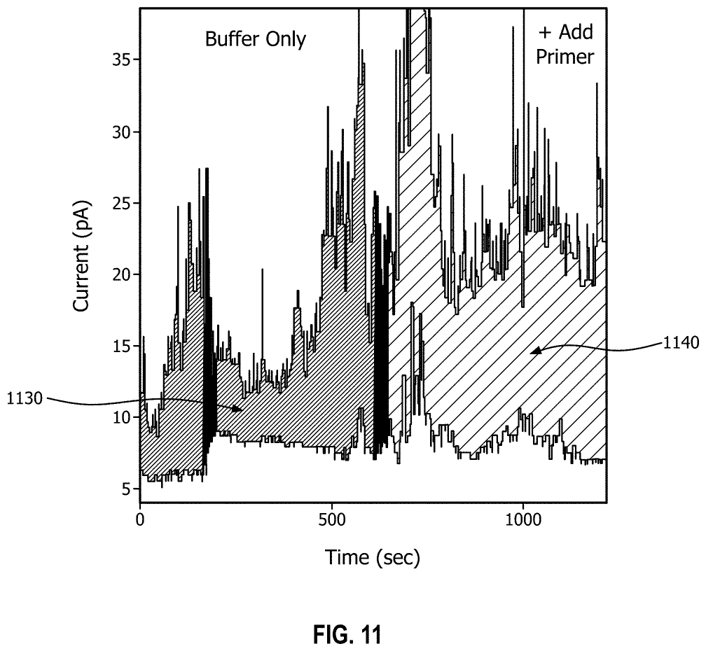

FIG. 11 shows a signal pixel trace obtained from the 19-mer hybridization probe sensor deployed on the 16 k pixel chip (FIG. 10A/B). In the region of the trace 1130 marked “Buffer Only,” the 14-mer hybridization target SEQ ID NO: 7 has not yet been introduced to the sensor. The target 14-mer SEQ ID NO: 7 is then added into the solution provided to the sensor, typically at a 1 μM concentration, giving rise to the signal trace portion 1140 marked “+Add Primer.” The signal trace reproduced in FIG. 11 covers approximately 1200 seconds of signal monitoring. Viewed at this high level, it is clear that more frequent and greater amplitude signal spikes occur once the primer is added to the sensor, as a result of primer hybridization events. The current levels and fluctuations observed are on the scale of about 20 to about 30 pA.

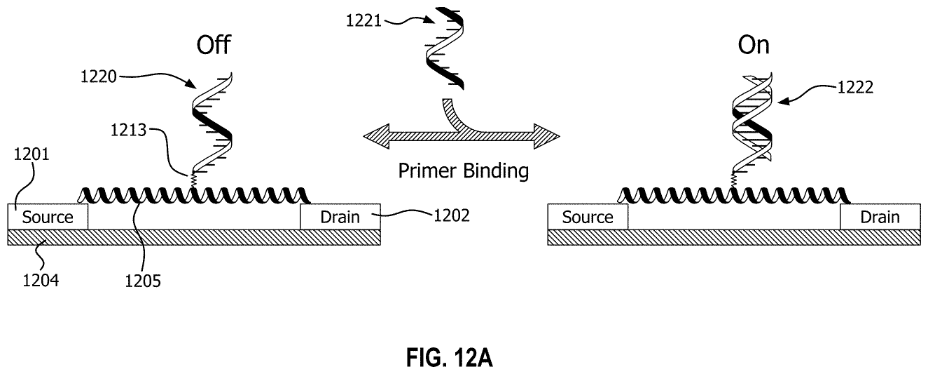

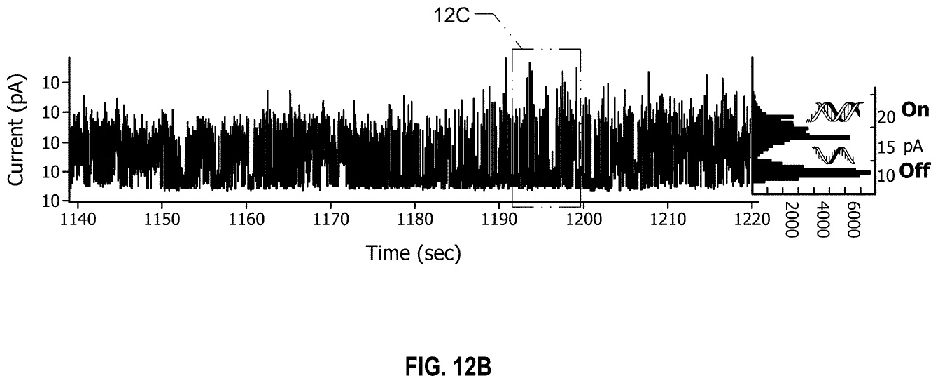

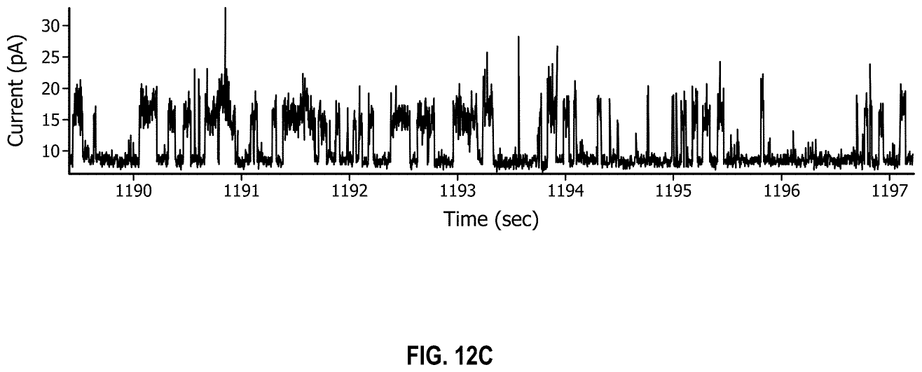

FIG. 12A reviews the general sensor structure used in the experiments. The molecular electronics sensor comprises a source electrode 1201 and a drain electrode 1202 spaced-apart from the source electrode 1201 by a nanogap. The two electrodes in the pair are disposed on a common substrate 1204. The electrodes are electrically connected by bridge molecule 1205 spanning the nanogap and bridging over the substrate 1204. The hybridization probe 1220 is attached to the bridge molecule 1205 by conjugation 1213, such as a linker, as described above. The “off” state of the sensor is shown at left in FIG. 12A. In use, the hybridization probe 1220 binds its target oligonucleotide 1221 (“primer binding”) to form the duplex 1222, which is denoted as the “on” state of the sensor shown in the structure at right in FIG. 12A.

FIGS. 12B and 12C show progressively expanded portions of the signal trace from the “+Add Primer” region of the trace in FIG. 11, with FIG. 12B showing the portion of the trace from about 1140 seconds to about 1220 seconds, and with FIG. 12C showing only an 8 second interval from about 1189 seconds to about 1197 seconds. In the expanded portion, two-level step ups to ˜15 pA are observed that are interpreted as the primer-bound or “on” states indicated in the sketch of FIG. 12A, while the baseline lower current state at ˜10 pA is interpreted as the “off” state as illustrated. The histogram of measured current values in FIG. 12B, from the 1 kHz sampling, shows that the current is distributed around these “on” and “off” levels, and provides a view of the relative time spent in each state. This trace illustrates that the sensor detects the basic hybridization of the 14-mer primer target, distinguishable as this “on”/“off” signal trace or signature, which differs from the noise seen in the “Buffer-Only” phase of the signal trace (FIG. 11). These results demonstrate the basic use of this device as a target sequence detector when configured as a hybridization sensor having a hybridization probe of complementary sequence.

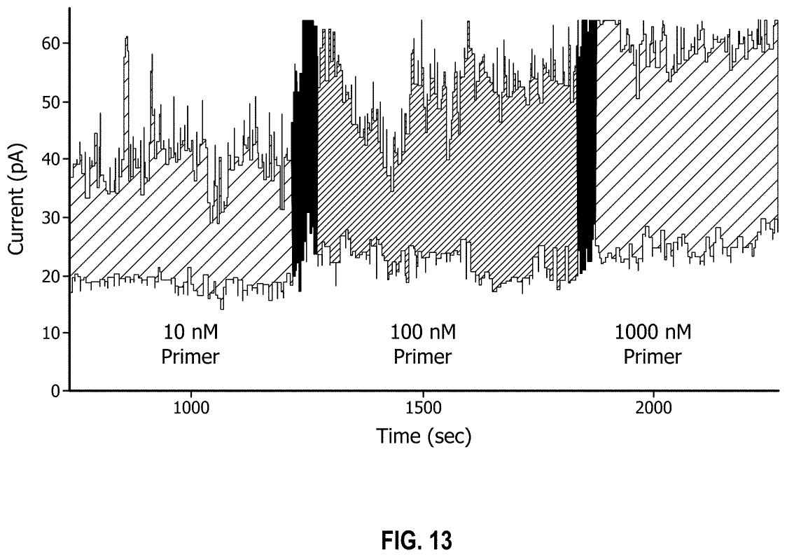

FIG. 13 shows a high level signal trace (current response) of the sensor on the chip when the concentration of target is raised from 10 nM to 100 nM and to 1000 nM (1 μM) over the course of about 1500 seconds. It is clear from examining the three portions of this signal trace in FIG. 13 that the nature of the signals, e.g., frequency of “on” state,” changes as the concentration of the target primer increases.

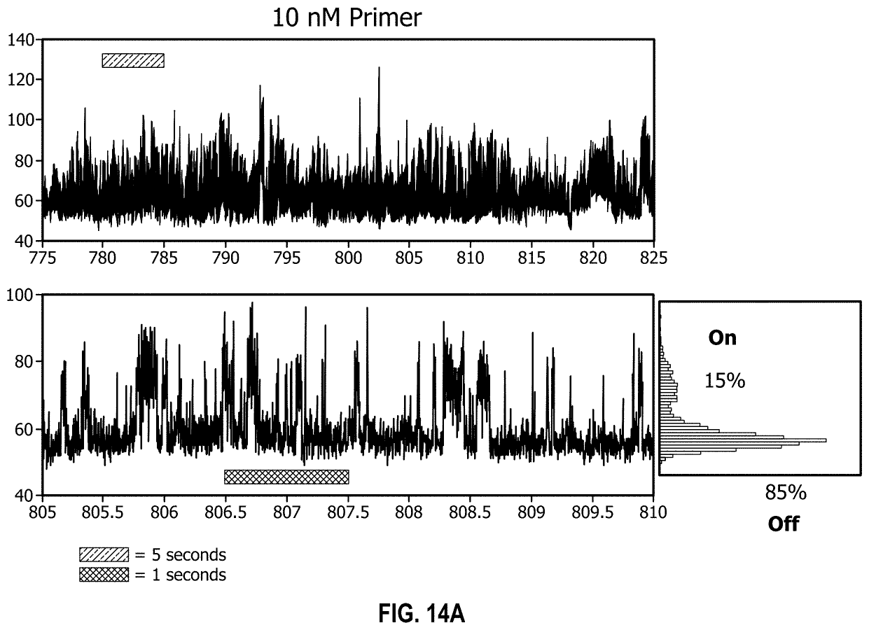

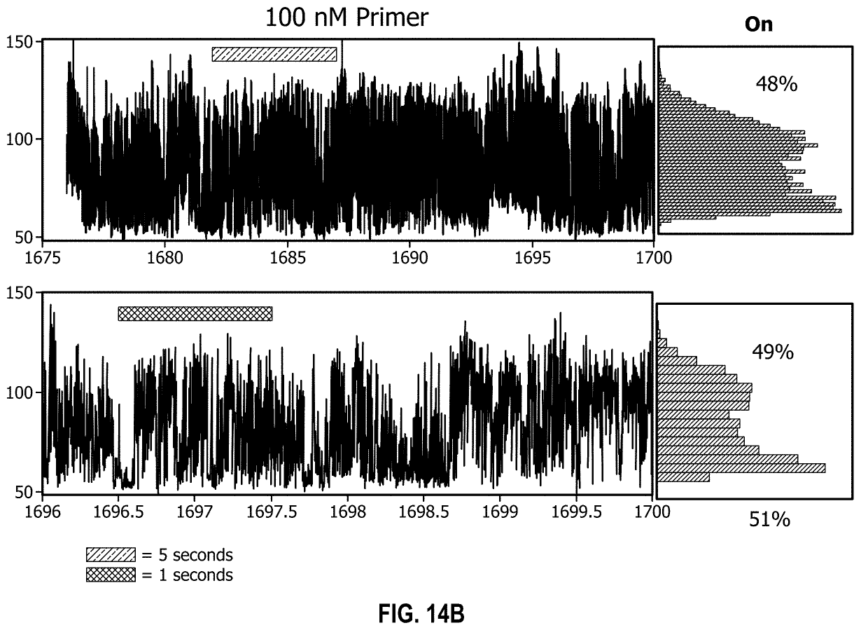

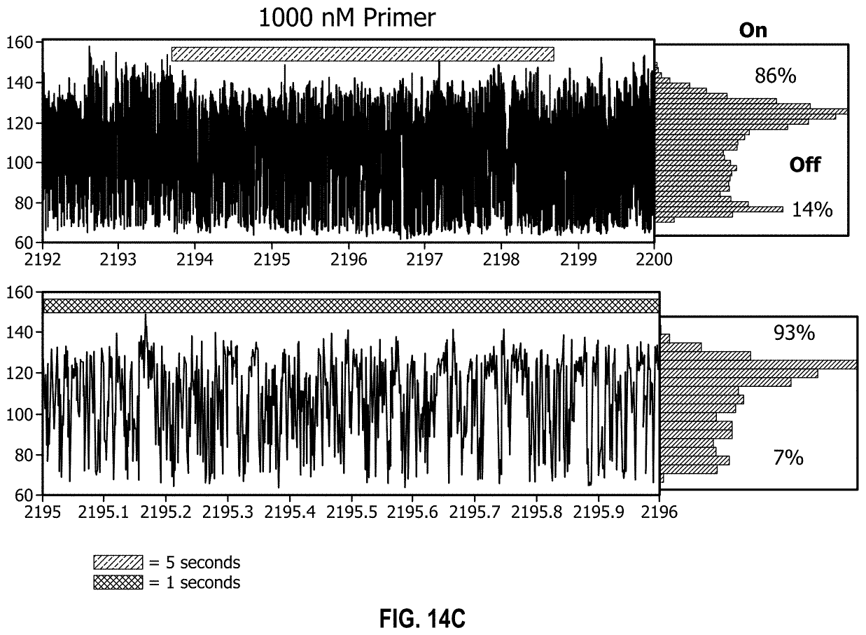

FIGS. 14A, 14B and 14C show signal traces and corresponding expanded regions of the signal traces obtained for the three concentrations of 14-mer primer, as well as histograms showing the relative times the primer/probe spent in the “on” (primer hybridized) and “off” states (primer and probe as separate strands). From the expanded signal traces, it is clear that the rate of signal spiking increases in frequency as the concentration of the primer target increases from 10 nM to 100 nM and to 1000 nM, and also that the relative proportion time the sensor is in the “on” state increases, while the amplitude of the current remains nearly constant. Thus, the variables such as pulse rate and “on” fraction correlate with concentration of the 14-mer primer target, clearly demonstrating that such a hybridization sensor can provide a measure of target concentration. This demonstrates the basic use of this sensor device to measure target concentration when the sensor configured as a hybridization sensor having a hybridization probe.

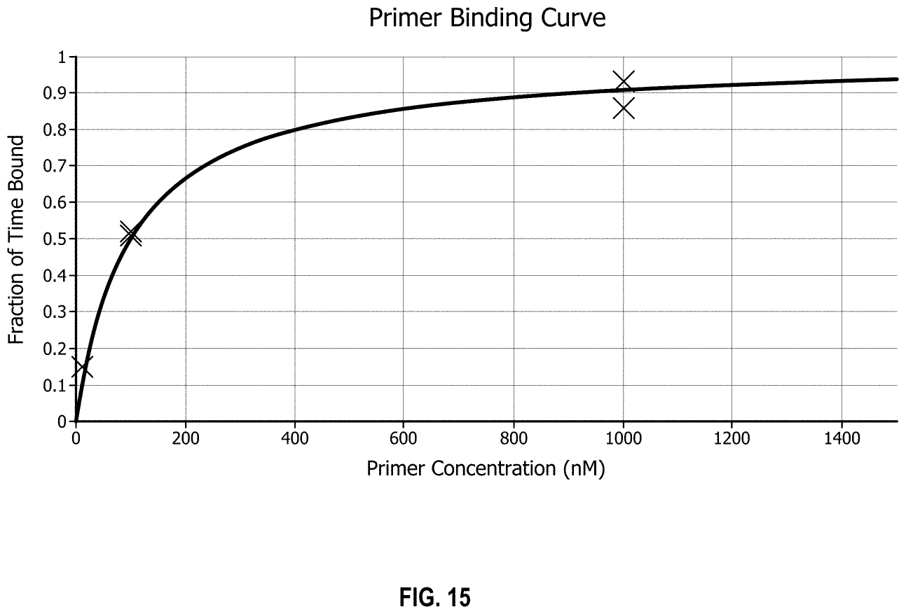

FIG. 15 illustrates a plot of “Fraction of Time Bound” versus Primer Concentration (nM), with a curve fit to the data points (shown as “X's,” at 10 nM, 100 nM, and 1000 nM). The data are fit to a standard exponential decay curve. The curve thus provides a calibration curve usable to obtain the concentration for a primary measurement such as “on” fraction. In other words, an “on” time measured in an experiment would be used on the calibration curve to interpolate the corresponding concentration of the target present.

FIG. 16 shows an experimental hybridization sensor configured with a longer oligonucleotide hybridization probe. In this case, the longer probe is a 45-mer having the following sequence:

(SEQ ID NO: 8)
5′-CGATCAGGCCTTCACAGAGGAAGTATCCTGTCGTTTAGCATACCC-
3′.

In order to conjugate the 5′-end of this oligonucleotide to the peptide bridge molecule having the amino acid sequence identified as SEQ ID NO: 3, an azide group was incorporated at the 5′-end of the oligonucleotide so as to participate in click chemistry conjugation. Further, a FAM dye molecule was attached to the 3′-end via the terminal hydroxyl group of the oligonucleotide.

Table 1 sets forth various primer targets, including perfect match and mismatch primer targets, which were used in various genetic analysis experiments with a sensor (per FIG. 10) comprising a hybridization probe having the oligonucleotide sequence identified as SEQ ID NO: 8, and further including the FAM dye molecule conjugated to the 3′-end.

TABLE 1
Primer Targets For Hybridization to SEQ ID NO: 8 Probe

| | | | Tₘ |
|---|---|---|---|
| Primer Name | Nucleotide Sequence | SEQ ID NO: | (° C.) |
| Full Complement | 3′-GCTAGTCCGGAAGTGTCTCCTTCATAGGACAGCAAATCGTATGGG-5′ | SEQ ID NO: 9 | — |
| Primer 2P-0 | 3′-GCTAGTCCGGAAGTGTCTCC-5′ Primer P-0 (20-mer) | SEQ ID NO: 10 | 57.6 |
| Primer 2P-1G | 3′-CTAGTCCGGAAGTGTCTCC-5′ Primer P-1G (19-mer) | SEQ ID NO: 11 | 55.1 |
| Primer 2P-2Cg | 3′-TAGTCCGGAAGTGTCTCC-5′ Primer P-2 (18-mer) | SEQ ID NO: 12 | 54.1 |
| Primer 2P-3Tcg | 3′-AGTCCGGAAGTGTCTCC-5′ Primer P-3 (17-mer) | SEQ ID NO: 13 | 53.9 |
| Primer 2P-4Atcg | 3′-GTCCGGAAGTGTCTCC-5′ Primer P-4 (16-mer) | SEQ ID NO: 14 | 52.4 |
| Primer 2P-5Gatcg | 3′-TCCGGAAGTGTCTCC-5′ Primer P-5 (15-mer) | SEQ ID NO: 15 | 50.6 |
| Primer 2P-5 | 3′-TCCGGAAGTGTCTCCTTCAT-5′ Primer P-10 (20-mer) | SEQ ID NO: 16 | — |
| Primer 2P-10 | 3′-AAGTGTCTCCTTCATAGGAC-5′ Primer P-15 (20-mer) | SEQ ID NO: 17 | — |
| Primer 2P-15 | 3′-TCTCCTTCATAGGACAGCAA-5′ Primer P-20 (20-mer) | SEQ ID NO: 18 | — |
| Primer 2P-20 | 3′-TTCATAGGACAGCAAATCGT-5′ Primer P-25 (20-mer) | SEQ ID NO: 19 | — |
| Primer 2P-25 | 3′-AGGACAGCAAATCGTATGGG-5′ Primer P-30 (20-mer) | SEQ ID NO: 20 | — |

With reference to Table 1, recall that it is the 5′-end of the hybridization probe (SEQ ID NO: 8) that is conjugated to the polypeptide bridge molecule, whilst the free 3′-end of the probe distal to the bridge molecule is conjugated to a FAM dye molecule (via the 3′-terminal hydroxyl group of the probe). In Table 1, the Tₘ is listed if it was determined, otherwise the Tₘ cell is left blank.

In Table 1, the Full Complement sequence is the perfect match target analyte to the probe. That is, this target analyte has a sequence that comprises the full complementary sequence to SEQ ID NO: 8. Primer 2P-0 is a 20-mer oligonucleotide that forms a partial duplex with the probe, beginning at the 3′-end at the bridge and extending for 20-bases toward the 5′-end with no mismatching. Primer 2P-1G is a 19-mer oligonucleotide that begins hybridization with the probe two bases in from the 3′-end, leaving the cysteine at the 3′-end of the probe unpaired. Similarly, Primer 2P-2Cg is an 18-mer oligonucleotide that begins hybridization with the probe three bases in from the 3′-end, leaving both the C and the G bases at the 3′-end of the probe unpaired. Similarly, Primer 2P-3Tcg is a 17-mer oligonucleotide that begins hybridization with the probe four bases in from the 3′-end, leaving the C, G and A bases at the 3′-end of the probe unpaired. Similarly, Primer 2P-4Atcg is a 16-mer oligonucleotide that begins hybridization with the probe five bases in from the 3′-end, leaving the C, G, A and T bases at the 3′-end of the probe unpaired. Similarly, Primer 2P-5Gatcg is a 15-mer oligonucleotide that begins hybridization with the probe six bases in from the 3′-end, leaving the C, G, A, T and C bases at the 3′-end of the probe unpaired.

With continued reference to Table 1, Primer 2P-5 is a 20-mer oligonucleotide that forms a partial duplex with the probe, beginning six bases in from the 3′-end at the bridge and extending for 20-bases toward the 5′-end with no mismatching. Primer 2P-10 is a 20-mer oligonucleotide that forms a partial duplex with the probe, beginning eleven bases in from the 3′-end at the bridge and extending for 20-bases toward the 5′-end with no mismatching. Primer 2P-15 is a 20-mer oligonucleotide that forms a partial duplex with the probe, beginning sixteen bases in from the 3′-end at the bridge and extending for 20-bases toward the 5′-end with no mismatching. Primer 2P-20 is a 20-mer oligonucleotide that forms a partial duplex with the probe, beginning twenty one bases in from the 3′-end at the bridge and extending for 20-bases toward the 5′-end with no mismatching. Lastly, Primer 2P-25 is a 20-mer oligonucleotide that forms a partial duplex with the probe, beginning twenty six bases in from the 3′-end at the bridge and extending for 20-bases to the 5′-end with no mismatching.

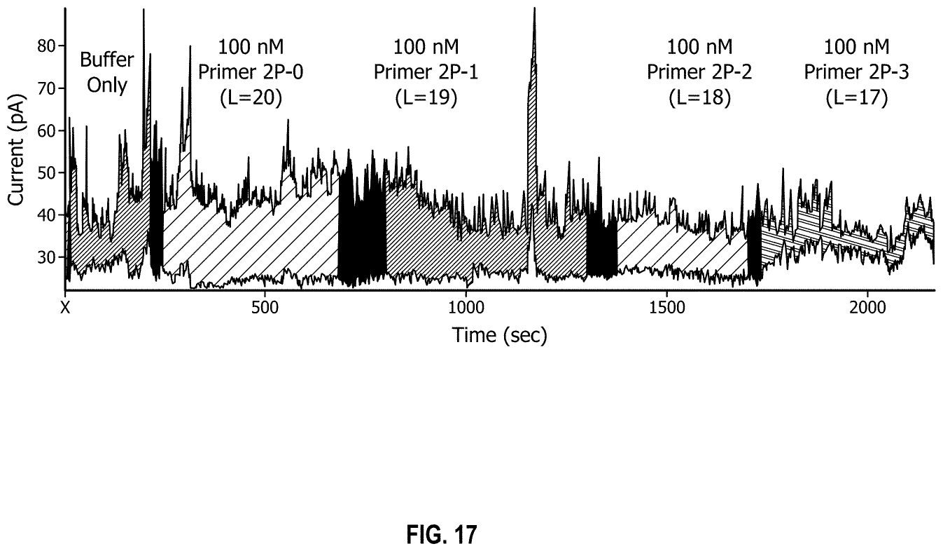

FIG. 17 shows a signal trace generated from the sensor of FIG. 15 (45-mer probe) on the 16 k chip over time, in an experiment in which the hybridization probe binds a series of progressively shorter targets, of length 20, 19, 18 and 17 bases. These targets are listed in Table 1, and consisted of Primer 2P-0, Primer 2P-1G, Primer 2P-2Cg and Primer 2P-3Tcg (in the signal trace of FIG. 17, these are more simply labeled as Primer 2P-0 (L=20), Primer 2P-1 (L=19), Primer 2P-2 (L=18) and Primer 2P-3 (L=17)). Each of the targets anchor (i.e., begin their complementary base pairing) at the same distal site of the probe molecule (i.e., beginning at the 10^{th} base in from the bridge, which is a G base), such that the 3′ end of the bound target, i.e., where the duplex ends, recedes away from the bridge peptide for the progressively shorter primer targets (and as discussed above in reference to Table 1). It can be seen in the high level trace that the signals are diminishing in both amplitude and frequency as the lengths of the target (and hence Tₘ) are reduced from the 20-mer, to the 19-mer, to the 18-mer and to the 17-mer target sequences.

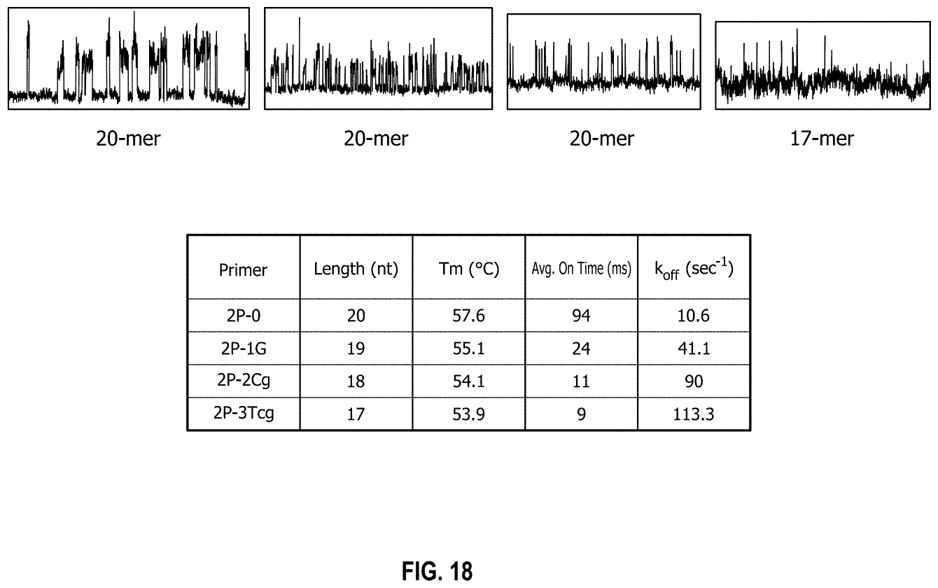

FIG. 18 shows expanded regions of the signal trace (from FIG. 17) in each phase of the experiment, showing that as the target oligonucleotide shortens (20-mer, 19-mer, 18-mer, and 17-mer, respectively), the amplitude of the current pulses drops only slightly. However, as shown in the table inset in FIG. 18, the average “on” time (duration of duplex hybridization, in ms units) drops off considerably (from 94 ms for the 20-mer target down to 9 ms for the 17-mer target), even though each target has no mismatches, just less bases to pair and a shifted position of the duplex. Further, the off-rate (k_{off}, in units of inverse seconds) increases dramatically as one progresses from the 20-mer target (k_{off}=10.6/sec) through to the 17-mer target (k_{off}=113.3/sec). Thus, the net effect of target sequence length on the signal trace is clearly observable and easily distinguishable. Overall, this experiment demonstrated good sensor sensitivity, as the sensor clearly detected 1-base level differences in the length of the target primers.

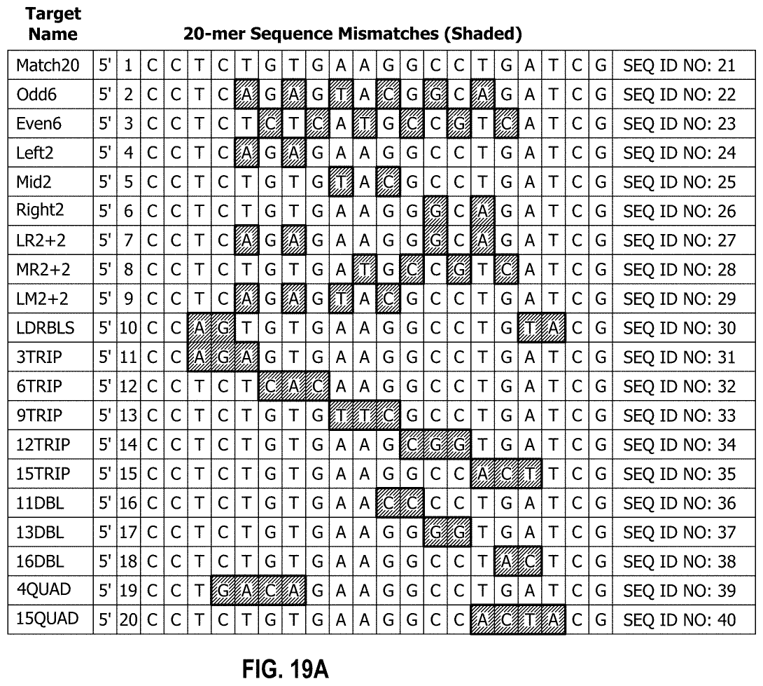

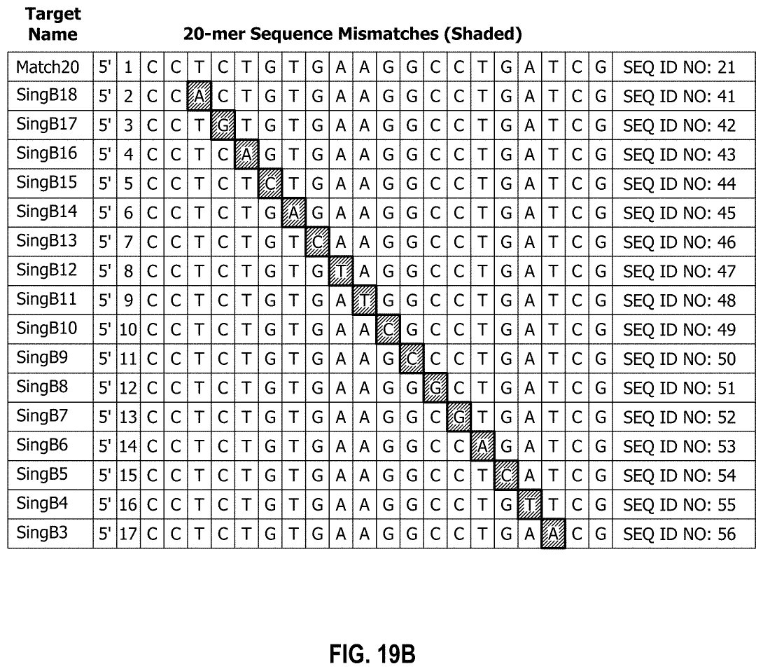

FIGS. 19A and 19B show a collection of mismatched primer targets having 1-base differences as well as multiple base differences from the perfect match target shown (labeled as Match20). FIG. 19A lists a series of 20-mer targets comprising multiple mismatches to the 45-mer probe sequence (FIG. 15), from 2 to 6 base mismatches, spaced out or adjacent. FIG. 19B lists various 20-mer targets, each with only a single mismatch to the 45-mer probe sequence (FIG. 15), moving down the length of the target sequence as shown.

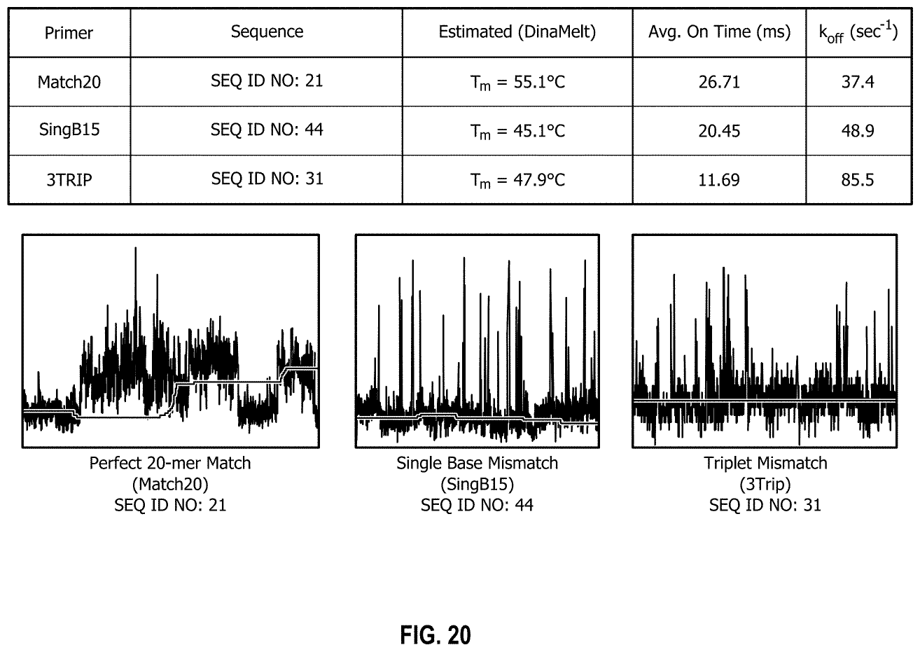

FIG. 20 shows signaling results obtained using the sensor configured with the 45-mer hybridization probe (FIG. 15) for 1-base (SingB15) and 3-base (3TRIP) mismatched target sequences, relative to the signaling observed for the perfect match (Match20) target sequence. The table inset in FIG. 20 also sets forth the respective Tₘ, the average “on” time, and corresponding “off” rates for each hybridization experiment. As shown in FIG. 20, the primer targets are Match20, having the oligonucleotide sequence 5′-CCTCTGTGAAGGCCTGATCG-3′ (SEQ ID NO: 21); SingB15, having the oligonucleotide sequence 5′-CCTCTCTGAAGGCCTGATCG-3′ (SEQ ID NO: 44); and 3TRIP, having the oligonucleotide sequence CCAGAGTGAAGGCCTGATCG (SEQ ID NO: 31). In comparing the three signal traces thus obtained for these targets, the signal trace obtained for the single base mismatch (SingB15) target is clearly changed in appearance from the signal trace obtained for the perfect match target (Match20), with lower “on” time as expected as well as reduced current signal amplitude. As shown, the triple mismatch target (3-base mismatch scenario, using 3TRIP as the target) produced a signal trace even further reduced in both “on” time and signal amplitude. These results show that a single base mismatch makes a substantial and detectible difference in signaling characteristics compared to a perfect match target sequence. This demonstrates the ability of this sensor to discriminate proper hybridization from cross-hybridization (e.g., the triple match target), which is critical to using this sensor to sense targets in complex pools of genetic material, and the single base discrimination enables applications such as SNP genotyping and pathogen strain determinations that depend on discerning single base differences in targets.

FIGS. 21A, 21B, 21C and 21D illustrate various embodiments of four types of molecular electronics sensor structures in accordance with the present disclosure. Each structure comprises a first electrode 2101, a second electrode 2102 spaced-apart from the first electrode by a nanogap as shown, disposed on a common substrate 2104. The sensor illustrated in FIG. 21A comprises a decoding hybridization probe 2116a conjugated by a linker 2114a to a site somewhere along the primary hybridization probe 2112a. The primary hybridization probe 2112a is conjugated to the bridge molecule 2105a at either its 3′ or 5′ end by the conjugation 2113a. The site along the primary hybridization probe 2112a where the decoding hybridization probe 2116a is conjugated may be chosen such that the decoding hybridization probe 2116a does not interfere with hybridizations between the primary hybridization probe 2112a and various target genetic materials presented in solution to the sensor.

The sensor illustrated in FIG. 21B comprises a decoding hybridization probe segment 2116b that is part of the primary hybridization probe 2112b conjugated to the bridge molecule 2105b by either its 3′ or 5′ end by conjugation 2113b. Stated another way, both the decoding hybridization probe and the primary hybridization probe of the sensor comprise a contiguous molecule. In various embodiments, the single molecule attached to the bridge may be an oligonucleotide, wherein the hybridization probe segment 2112b comprises one oligonucleotide sequence of the oligonucleotide and the decoding hybridization probe 2116b comprises another oligonucleotide sequence of the overall oligonucleotide, wherein the two sequences may be directly continuous or separated, such as by one or more intervening bases. For example, one or more intervening nucleotides between the primary hybridization segment and the decoding hybridization probe segment may remain unpaired when the sensor is exposed to various genetic materials in use for genetic analysis. In other embodiments, a single molecule may have other chemical separation between the two oligonucleotide segments, e.g., methylene groups, ethylene oxide groups, etc., disposed between the two oligonucleotide segments of the same molecule. In various embodiments, a single molecule attached to the bridge may comprise any number of decoding hybridization segments, and these may be contiguous or separated along the single molecule that also includes the primary hybridization sequence.

The sensor illustrated in FIG. 21C comprises two decoding hybridization probes 2117 and 2119, each conjugated by a linker (2118 and 2120, respectively) to two site along the primary hybridization probe 2112c. The two sites of conjugation between the primary hybridization probe 2112c and the two decoding hybridization probes 2117/2119 can be spaced apart as desired along the primary hybridization probe 2112c, e.g., so as not to interfere with the ability of the primary hybridization probe to hybridize with a target molecule. In various embodiments, there may be more than two decoding probes conjugated to the primary hybridization probe at any sites along the primary hybridization probe.

The sensor illustrated in FIG. 21D comprises a single molecule (as per structure FIG. 21B), but where the molecule having both the decoding probe 2116d and the primary hybridization probe 2112d is conjugated from an internal position, rather than from one end, to the bridge molecule 2105d via conjugation 2113d. Advantages to this structure (D) include ensuring that either or both of the primary hybridization probe and the decoding hybridization probe sequence remain physically unhindered for hybridizing. For example, the conjugation of such a strand to the bridge molecule could be at an interior part of the strand in order to allow both the hybridization probe segment and the one or more decoding target segments to be similarly close to the bridge for more sensitive detection of the decoding targets.

FIGS. 22A, 22B, 22C and 22D illustrates additional embodiments of molecular electronics sensor structures in accordance with the present disclosure. Each structure comprises a first electrode 2201, a second electrode 2202 spaced-apart from the first electrode 2201 by a nanogap as shown, and a bridge molecule coupling the two electrodes. The two electrodes are disposed on a common substrate 2204.

FIG. 22A illustrates a sensor structure wherein the attachment of the hybridization probe 2212a to the primary hybridization probe is by a long linker 2114a to the bridge molecule 2205a, and wherein the linker is conjugated to the bridge molecule 2205a via the conjugation 2213a. FIG. 22B illustrates a sensor structure comprising a single DNA strand conjugated to the bridge molecule 2205b via conjugation 2213b, wherein a sub-segment 2212b of the single DNA strand comprises a sequence specific for a target, with additional sequences 2208 and 2209 at one or both ends. FIG. 22C illustrates a sensor structure comprising two hybridization probes, 2217 and 2219, conjugated by linkers 2218 and 2220, respectively, to a conjugation site 2213c on the bridge molecule 2205c. FIG. 22D illustrates a sensor structure wherein the hybridization probe 2212d is attached to the bridge molecule 2205d at an internal site of the probe strand, via conjugation 2213d. As suggested by the structures in FIG. 22A-22D, the hybridization probe-containing strand may be attached in diverse manners to the bridge molecule, e.g., including multiple points of attachment, or having a non-linear form, such as the branched forms in FIGS. 21A and 21C, or in a circular form, or other diverse forms, so long as such structural forms allow the portion of the strand comprising a complementary sequence to a sequence of the target to be available to form a hybridized duplex with the target strand.

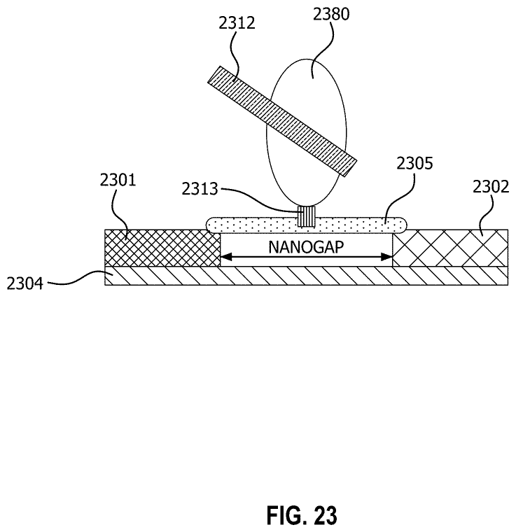

FIG. 23 illustrates an additional embodiment of a molecular electronics sensor structure in accordance with the present disclosure, wherein an additional intermediary complexing molecule 2380 may be used to complex the hybridization probe 2312 with the bridge 2305. Such a molecule 2380 could be a larger protein that complexes with DNA, such as a DNA binding protein, or a DNA binding enzyme such as a polymerase, ligase, restriction enzyme, CRISPR/CAS9 protein. As illustrated in FIG. 23, the sensor comprises a first electrode 2301, a second electrode 2302 spaced-apart from the first electrode 2301 by a nanogap, and a bridge molecule 2305 coupled to both electrodes. The complexing molecule 2380 may be conjugated to the bridge 2305 by conjugation 2313. The pair of electrodes are disposed on a common substrate 2304. In various embodiments, an intermediate molecule 2380 that couples the hybridization probe 2312 to the bridge molecule 2305 can enhance the signal produced by the various hybridization events involving the hybridization probe 2312.

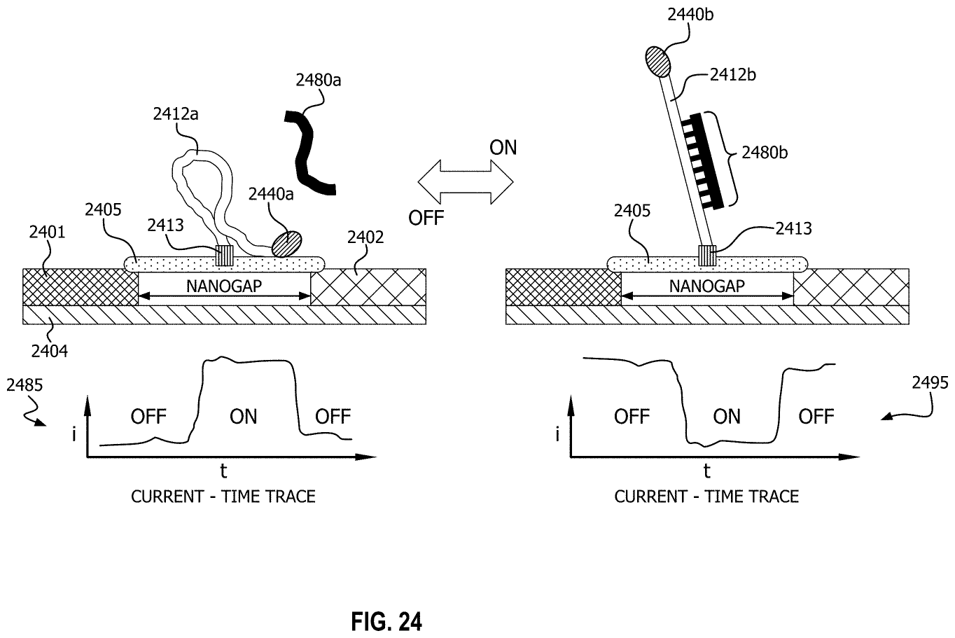

FIG. 24 illustrates additional embodiments of molecular electronics sensor structures in accordance with the present disclosure, wherein the single stranded probe 2412a may have a secondary structure (e.g., folding onto itself) that is stable and preferentially adopted and/or where the probe may have a conformation that interacts with the bridge (e.g., as shown in the structure at left). The sensor comprises a first electrode 2401, a second electrode 2402 spaced-apart from the first electrode 2401 by a nanogap as illustrated, and a bridge molecule 2405 coupled to the two electrodes that are disposed on a common substrate 2404. In various embodiments, the secondary conformation of the hybridization probe 2412a, e.g., illustrated in the structure at left, unfolds to open structure 2412b to hybridize with the target 2480a, giving rise to the structure at right where the secondary structure is gone and a probe-target duplex 2480b results from hybridization of probe 2412b and target 2480a. In various embodiments, there may also be a signaling group 2440a on the probe, e.g., at the free end distal to the bridge molecule, which may produce a detectible signal when it changes configuration between the “on” and “off” states of hybridization. For example, the signaling group may move from a position 2440a where it is in contact with the bridge molecule (in the “off” state with regards to duplex formation) to a position 2440b where it is no longer in contact with the bridge molecule 2405 (in the “on” state where the probe is extended and hybridized to the target sequence). In general, the secondary structure preferably has a melting point Tₘ near or slightly below that of the exact match hybridization, so that the exact match can compete with some efficiency for binding by displacement of the secondary structure, but other fragments that do not match, have greatly reduced ability to form stable interactions with the probe as they cannot compete with the stability of the secondary structure of the probe. The changes in the secondary structure, included optionally a change in the location of the signaling group on the probe, can be used to produce a detectible signal, or a larger signal, or greater signal-to-noise in detecting hybridization, as illustrated in the two current-time traces 2485 and 2495. The secondary structure of the probe 2412a may also decrease the signals, or the noise, from off-target strands present in solution. In various embodiments, the Tₘ of the secondary structure may be about 5° C. to about 10° C. below the target-probe duplex Tₘ, or about 1° C. to about 5° C. below, or within 1° C. of, or about 1° C. to about 5° C. degrees above this Tₘ, or in various embodiments, about 5° C. to about 10° C. above this Tₘ. In various embodiments, all or part of the secondary structure may be designed or induced by DNA hybridization bonding. In various embodiments, this is done through the use of matched and mismatched base pairing. Such secondary structure elements may include hairpin formation.

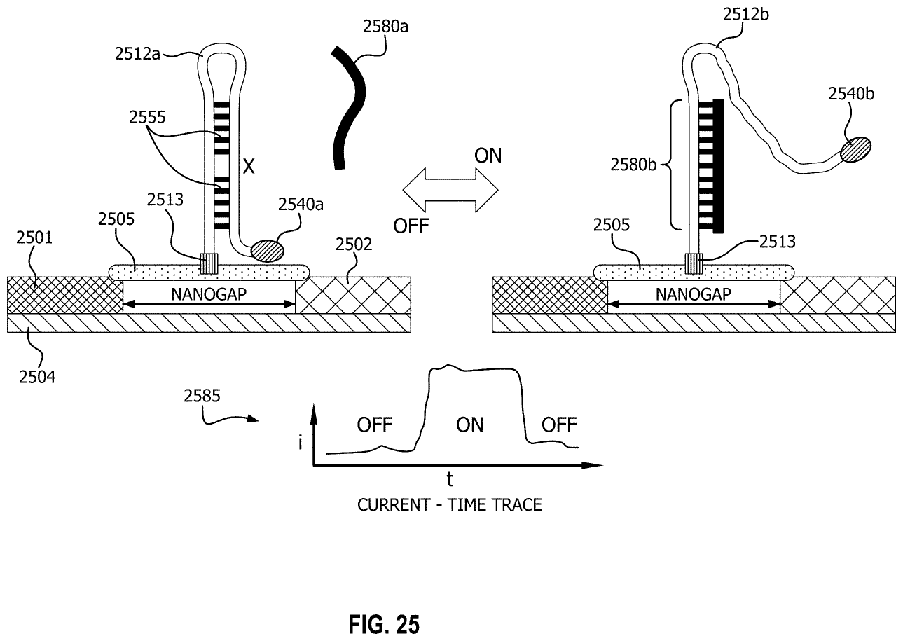

As illustrated in FIG. 25, the hybridization probe 2512a may be extended in length to include a segment that is capable of forming a hairpin with one or more mismatches 2555 to the probe segment specific to the target of the hybridization. The sensor comprises a first electrode 2501, a second electrode 2502 spaced-apart from the first electrode 2501 by a nanogap as illustrated, and a bridge molecule 2505 coupled to the two electrodes that are disposed on a common substrate 2404. In the hairpin configuration illustrated in the structure at left, the target molecule 2580a can bind to the probe with high stringency against unwanted off-target hybridization. The hairpin also affords the option of having a signaling group 2540a on the free end of the hairpin probe, such that in the hairpin form, the signaling group 2540a will be held near or in contact with the bridge 2505, and in the “on” state where the probe is bound to its hybridization target, this signaling group 2540b will be displaced relatively far from the bridge molecule 2505, and will become more mobile, both effects contributing to an observable signal characteristic of hybridization.

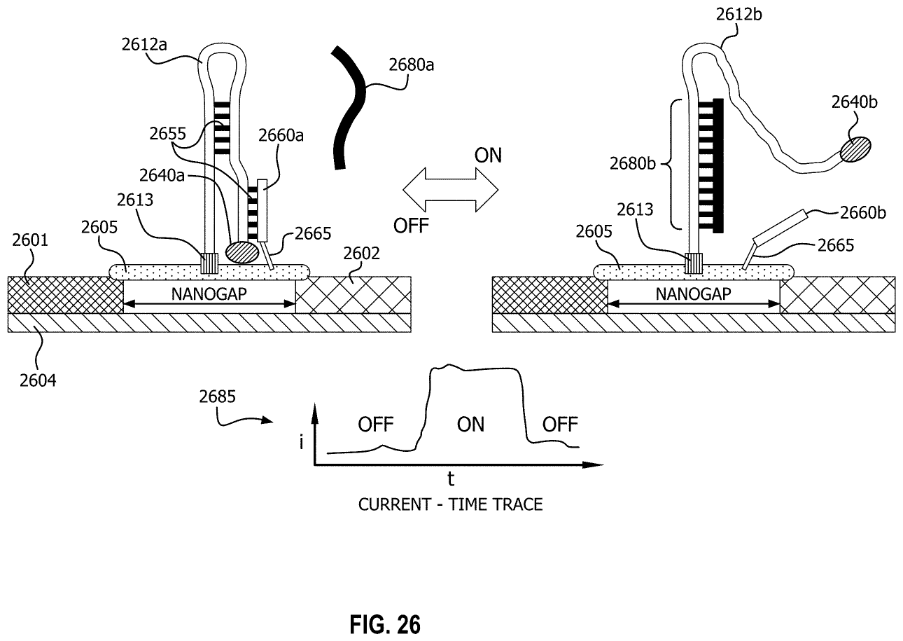

FIG. 26 illustrates another embodiment of a sensor structure, where the hybridization probe 2612a (conjugated to the bridge 2605 by conjugation 2613) comprises a hairpin secondary configuration (with duplex regions 2655 as indicated) and the probe also binds to a secondary oligonucleotide 2660a that is conjugated to the bridge molecule 2605. The sensor comprises a first electrode 2601, a second electrode 2602 spaced-apart from the first electrode 2601 by a nanogap as illustrated, and a bridge molecule 2605 coupled to the two electrodes that are disposed on a common substrate 2604. The secondary oligonucleotide 2660a attached to the bridge 2605 by linker 2665 acts as a latch to hold in the end of the probe 2612a carrying the signaling group 2640a. The target sequence 2680a preferentially binds to the probe 2612a, thus opening the latch 2660b/2665 and freeing the signaling group 2640b. This configuration may also be used to enhance a hybridization event signal, either from the disruption of this secondary hybridization to a bridge oligonucleotide, or by changes in location of the signaling group once freed from latching effect of the bridge oligonucleotide.

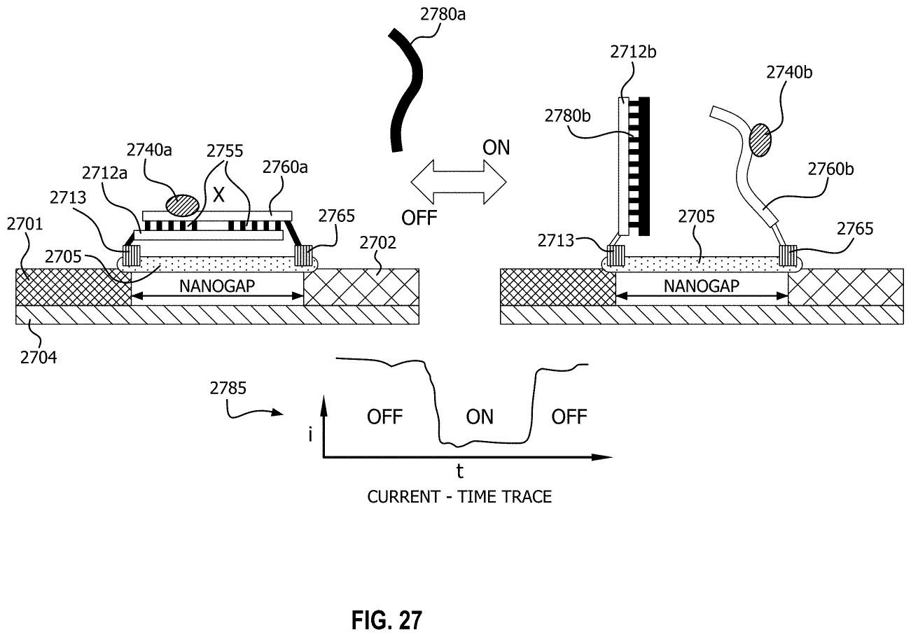

FIG. 27 illustrates an embodiment of a sensor structure that begins in the “off” state with parallel bridge structures, one permanent and one temporary. The sensor comprises a first electrode 2701, a second electrode 2702 spaced-apart from the first electrode 2701 by a nanogap as illustrated, and a bridge molecule 2705 coupled to the two electrodes that are disposed on a common substrate 2704. The hybridization probe 2712a is conjugated to the bridge 2705 by conjugation 2713. A protection strand 2760a is conjugated to the bridge 2705 via the conjugation 2765. The primary hybridization strand 2712a and the protection strand 2760a can participate in hybridization to form a duplex having partial base pairing at 2755 as shown, and this duplex resides as a second bridge structure over the permanent bridge 2705. Optionally, the protection strand 2760a includes a signaling group 2740a that can enhance signaling. When the sensor encounters a target sequence 2780a, the partial duplex opens and the primary hybridization probe 2712a hybridizes with the target 2780a to produce the duplex 2780b shown in the structure at right. This effect is competitive hybridization, wherein the target binding is stronger than the binding in the temporary partial duplex in the “off” state. When the temporary bridge structure opens, the permanent bridge 2705 still remains, and the optional signaling group 2740b is freed, enhancing the signal. As shown in the inset current-time trace 2785, the “on” state is characterized by less current since the double bridge structure is reduced to just a single bridge structure.

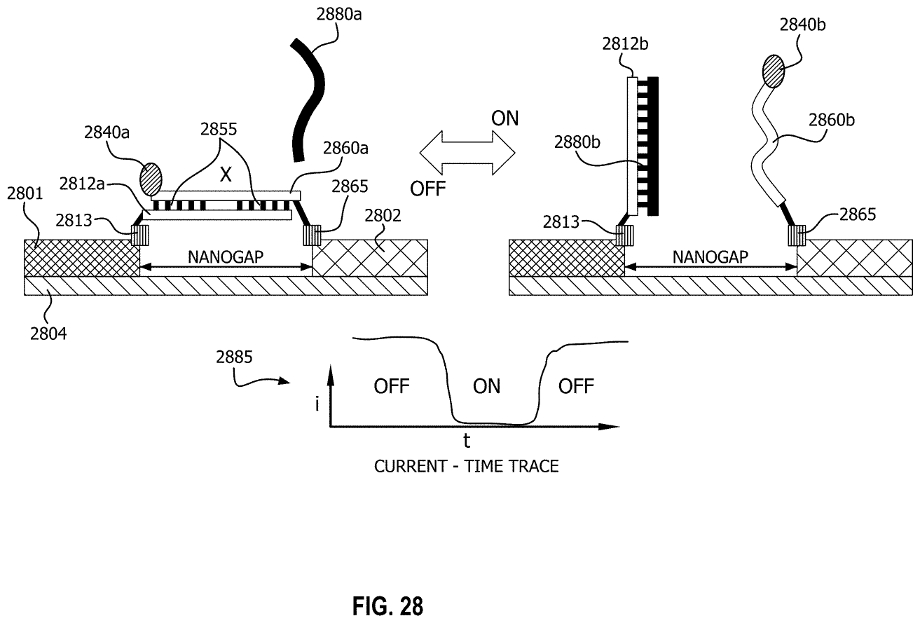

FIG. 28 illustrates another embodiment of a sensor structure, where a protection strand 2860a is used, which binds the target 2880a less effectively than the target 2880a can, and differs by mismatches from the exact match target for finer control of this effect. The protection strand 2860a, comprising a single strand oligonucleotide, is conjugated to the second electrode 2802 by conjugation 2865 and positioned as indicated so that it creates a partial duplex 2855 with the primary hybridization probe strand 2812a conjugated to the first electrode 2801 via the conjugation 2813. The duplex structure creates a temporary bridge and a closed circuit configuration between electrodes. An enhancement in signaling may also be afforded by the presence of a signaling group 2840a attached to the protection strand 2860a, which moves when the protection strand is displaced by hybridization 2880b between probe 2812b and target 2880a. Upon hybridization to the target strand, the temporary parallel bridge is disrupted, the circuit opens (no current in the current-time trace 2885) and the signaling group 2840b moves to create other effects such as optical signaling.

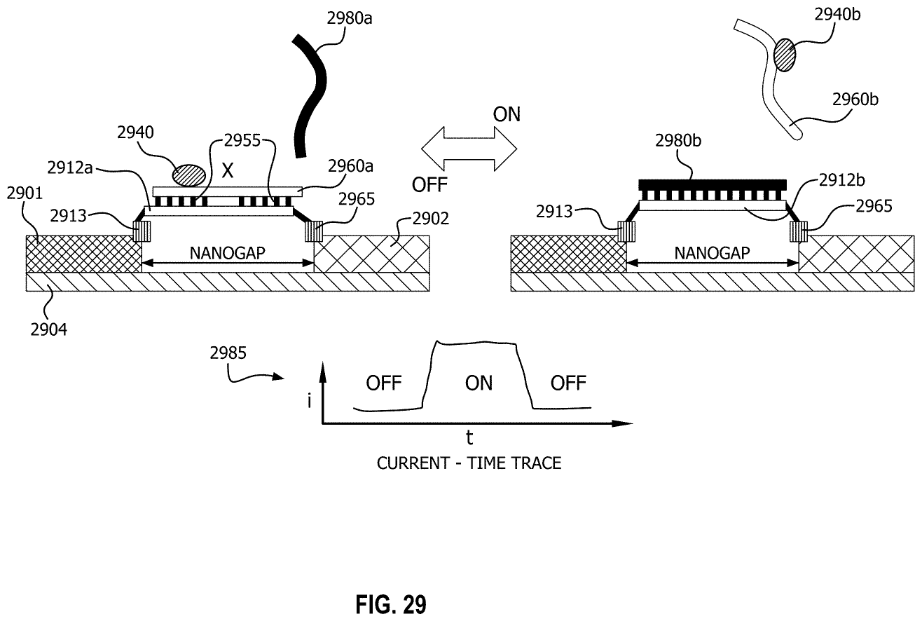

FIG. 29 shows an embodiment of a sensor structure, where a protection strand used to form a temporary bridge structure is entirely displaced from the sensor when the sensor binds a target sequence. The sensor comprises a first electrode 2901, a second electrode 2902 spaced-apart from the first electrode 2901 by a nanogap as illustrated. The hybridization probe 2912a is conjugated to the first electrode 2901 by conjugation 2913. A protection strand 2960a is conjugated to the second electrode 2902 via the conjugation 2965. The primary hybridization strand 2912a and the protection strand 2960a can participate in hybridization to form a duplex having partial base pairing at 2955 as shown, and this duplex forms a bridge structure connecting the first and second electrodes. The bridge formed by the protection strand is a mismatched duplex or otherwise a lower Tₘ duplex (lower, relative to target Tₘ). In various embodiments, a signaling group 2940a may be included on the protection strand 2960a. In this case, upon hybridization to the target 2980a, the protection strand 2960b is displaced and lost in solution along with its attached signaling group 2940b, and a complete duplex is formed between probe 2912b and target 2980b as shown at right for the “on” state. These processes may result in a detectible signal.

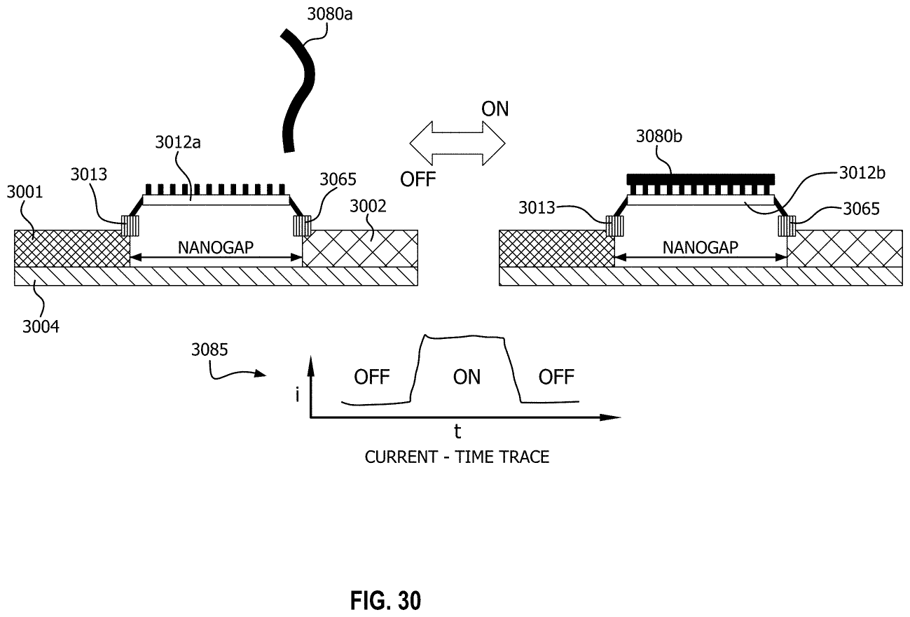

FIG. 30 illustrates another embodiment of a sensor structure, where the single stranded hybridization probe spans the electrodes as a bridge, and where binding of the target to the probe bridge creates a duplex DNA bridge, which may produce a signal comprising a large jump in baseline current to much higher current levels. The sensor comprises a first electrode 3001, a second electrode 3002 spaced-apart from the first electrode 3001 by a nanogap as illustrated. The single stranded hybridization probe 3012a, rather than being conjugated to a bridge molecule at one end, is conjugated at its 3′ and 5′-ends to each of the first 3001 and second 3002 electrodes. The target sequence 3080a forms a duplex with the hybridization prove 3012b as indicated in the structure at right. Since the bridge is transformed from a single stranded bridge to a double stranded bridge, conductivity increases as shown by the increase in current amplitude in the current-time trance 3085 for the “on” state of the sensor.

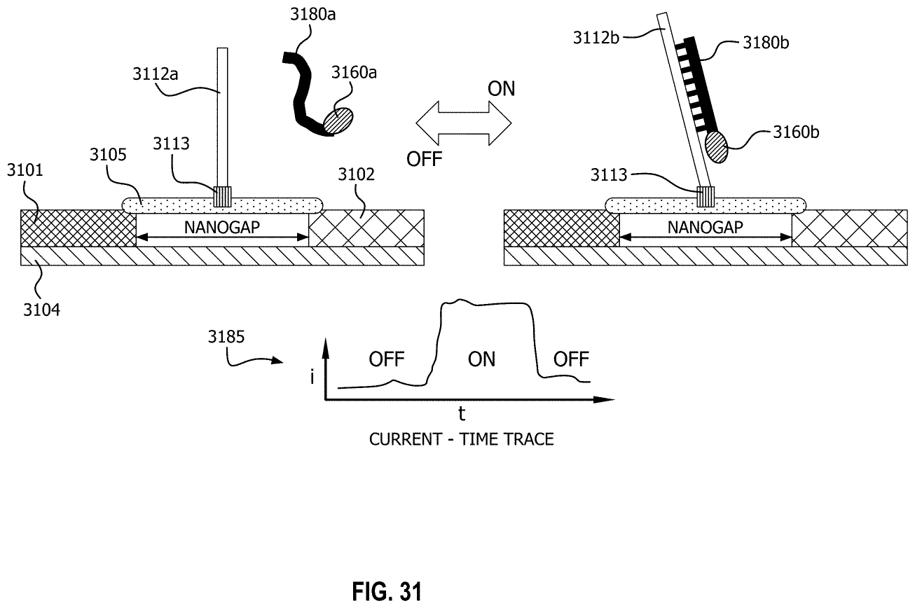

FIG. 31 illustrates another embodiment of a sensor structure, where the target strand 2980a has an added signaling group 2960a, resulting in a larger signal from the hybridization event that forces this group in close proximity to the bridge molecule 2905. In this embodiment, the sensor comprises a first electrode 3101, a second electrode 3102 spaced-apart from the first electrode 3101 by a nanogap, and a bridge molecule 3105 coupled to the two electrodes. The two electrodes are disposed on a common substrate 3104. The primary hybridization probe 2912a is coupled to the bridge molecule 3105 by the conjugation 3113. When the hybridization probe 3112a hybridizes with the target sequence 2980a, the signaling group 2960b is forced into close proximity to the bridge molecule and is fixed in that position during the “on” state of the sensor. Such a group may be added to the sample by standard sample labelling reactions that are commonly used to attach diverse labels to DNA or RNA. It is important to note that target analytes having a signaling group label can be used with any the hybridization sensor embodiments and applications disclosed here.

Multiplexed Sensor Array Configurations

As discussed herein, sensor systems in accordance with the present disclosure can be configured to identify the presence of and the identity of various strains of organisms in a sample, such as pathogens in a bio-sample. Sensor systems herein can be configured to test the presence of, and the response to, various viral and bacterial diseases.

In various embodiments, multiplexed sensor arrays, comprising a plurality of sensor pixels as explained herein, are configured such that a first subset of sensor pixels detects a first pathogen, a second subset of sensor pixels detects a second pathogen, a third subset of sensor pixels detects a third pathogen and so forth, up to as many subsets as necessary for a particular sensor application. In various embodiments, at least one subset of sensor pixels may be configured as control sensors. For example, one subset of sensor pixels may be configured as a control, such as to ensure the bio-sample is of human origin. The sum of the subsets present in an array equals the plurality (the total) of sensor pixels. A sensor array having any number of pixel subsets may be configured on a CMOS chip.

In various embodiments, a plurality of sensor pixels in a sensor array comprises at least one subset of sensor pixels. In various embodiments, the at least one subset of sensor pixels is configured to detect genetic material relating to a pathogen. In various embodiments, the pathogen may be, for example, SARS-CoV-2, Influenza A1, Influenza A2, Influenza B, or Respiratory Syncytial Virus (RSV).

In various embodiments, a plurality of sensor pixels in a sensor array comprises at least two subsets of sensor pixels. In various embodiments, one subset in the at least two subsets of sensor pixels is configured to detect genetic material relating to a pathogen. In various embodiments, the pathogen may be, for example, SARS-CoV-2, Influenza A1, Influenza A2, Influenza B, or Respiratory Syncytial Virus (RSV). In various embodiments, a second subset in the at least two subsets of sensor pixels comprises sensor pixels configured as control sensors. In various embodiments, a control may be, for example, a loading control or a sample origin control (e.g., to ensure that a bio-sample actually came from a human).

In various embodiments, a subset of sensor pixels in a plurality of sensor pixels comprises a DNA sequence complementary to a segment of a pathogen genome. For example, a plurality of sensor pixels in an array may comprise multiple subsets of sensor pixels, wherein a first subset comprises hybridization probes having a DNA sequence complementary to a segment of a first pathogen genome, a second subset comprises hybridization probes having a DNA sequence complementary to a segment of a second pathogen genome, a third subset comprises hybridization probes having a DNA sequence complementary to a segment of a third pathogen genome, and so forth. These pathogens may include, for example, SARS-CoV-2, Influenza A1, Influenza A2, Influenza B, or Respiratory Syncytial Virus (RSV). One of the subsets out of the multiple subsets present in the plurality may be configured as a control. In various embodiments, a subset of sensor pixels within a plurality of sensor pixels comprises hybridization probes with a DNA sequence complementary to a segment of a constitutively expressed human gene.

In various embodiments, an array comprises a plurality of sensor pixels further comprising two subsets of sensor pixels, a first subset and a second subset of pixels. The first subset of sensor pixels within the plurality comprises hybridization probes having a DNA sequence complementary to a segment of a pathogen genome. The second subset of sensor pixels within the plurality comprises hybridization probes with a DNA sequence complementary to a segment of a constitutively expressed human gene.

In various embodiments, an array comprises a plurality of sensor pixels further comprising three subsets of sensor pixels, a first subset, a second subset and a third subset of pixels. The first subset of sensor pixels within the plurality comprises hybridization probes having a DNA sequence complementary to a segment of a first pathogen genome. The second subset of sensor pixels within the plurality comprises hybridization probes having a DNA sequence complementary to a segment of a second pathogen genome. The third subset of sensor pixels within the plurality comprises hybridization probes with a DNA sequence complementary to a segment of a constitutively expressed human gene.

In various embodiments, an array comprises a plurality of sensor pixels further comprising four subsets of sensor pixels, a first subset, a second subset, a third subset and a fourth subset of pixels. The first subset of sensor pixels within the plurality comprises hybridization probes having a DNA sequence complementary to a segment of a first pathogen genome. The second subset of sensor pixels within the plurality comprises hybridization probes having a DNA sequence complementary to a segment of a second pathogen genome. The third subset of sensor pixels within the plurality comprises hybridization probes having a DNA sequence complementary to a segment of a third pathogen genome. The fourth subset of sensor pixels within the plurality comprises hybridization probes with a DNA sequence complementary to a segment of a constitutively expressed human gene.

In various embodiments, an array comprises a plurality of sensor pixels further comprising five subsets of sensor pixels, a first subset, a second subset, a third subset, a fourth subset and a fifth subset of pixels. The first subset of sensor pixels within the plurality comprises hybridization probes having a DNA sequence complementary to a segment of a first pathogen genome. The second subset of sensor pixels within the plurality comprises hybridization probes having a DNA sequence complementary to a segment of a second pathogen genome. The third subset of sensor pixels within the plurality comprises hybridization probes having a DNA sequence complementary to a segment of a third pathogen genome. The fourth subset of sensor pixels within the plurality comprises hybridization probes having a DNA sequence complementary to a segment of a fourth pathogen genome. The fifth subset of sensor pixels within the plurality comprises hybridization probes with a DNA sequence complementary to a segment of a constitutively expressed human gene. In variations of this configuration, the sensor array is capable of detecting and distinguishing between SARS-CoV-2, an Influenza A strain, an Influenza B strain, and Respiratory Syncytial Virus (RSV), and capable of confirming that a bio-sample thus analyzed is of human origin.

In various embodiments, an array comprises a plurality of sensor pixels further comprising six subsets of sensor pixels, a first subset, a second subset, a third subset, a fourth subset, a fifth subset and a sixth subset of pixels. The first subset of sensor pixels within the plurality comprises hybridization probes having a DNA sequence complementary to a segment of a first pathogen genome. The second subset of sensor pixels within the plurality comprises hybridization probes having a DNA sequence complementary to a segment of a second pathogen genome. The third subset of sensor pixels within the plurality comprises hybridization probes having a DNA sequence complementary to a segment of a third pathogen genome. The fourth subset of sensor pixels within the plurality comprises hybridization probes having a DNA sequence complementary to a segment of a fourth pathogen genome. The fifth subset of sensor pixels within the plurality comprises hybridization probes having a DNA sequence complementary to a segment of a fifth pathogen genome. The sixth subset of sensor pixels within the plurality comprises hybridization probes with a DNA sequence complementary to a segment of a constitutively expressed human gene. In variations of this configuration, the sensor array is capable of detecting and distinguishing between SARS-CoV-2, Influenza A1, Influenza A2, Influenza B, and Respiratory Syncytial Virus (RSV), and capable of confirming that a bio-sample thus analyzed is of human origin.

Any number of subsets of sensor pixels in a plurality of sensor pixels is within the scope of the present disclosure, as is any number of subsets of sensor pixels within an array that are dedicated to control functions, such as loading controls or sample origin controls. In various embodiments, any one or more of the subsets of sensor pixels present in a plurality of sensor pixels may have molecular electronics sensors further comprising decoding probes, such as conjugated to each hybridization probe in the subset.

In various embodiments, a subset of sensor pixels in a plurality of sensor pixels comprises hybridization probes having a DNA sequence complementary to a segment of β-actin human gene. In various embodiments, the hybridization probes in the control subset may have a DNA sequence complementary to a segment of any other known housekeeping gene, such as GAPDH or ubiquitin.

In various embodiments, a subset of sensor pixels in a plurality of sensor pixels comprises hybridization probes having a DNA sequence complementary to a segment of human RNase P gene, the gene coding for the RNA subunit of ribonuclease P (RNase P) in humans.

Table 2 sets forth various primers and probe sequences according to the present disclosure.

TABLE 2
Primer and Probe Sequences

| | | | | | |
|---|---|---|---|---|---|
| Assay | Name | Left primer (for) | Right primer (rev) | Probe | Ref* |

Influenza

| | | | | | |
|---|---|---|---|---|---|
| CDC | InfA_1 | CAAGACCAATCYT | GCATTYTGGACAAA | 5′-/5FAM/TGC AGT CCT CGC | 1 |
| | | GTCACCTCTGAC | VCGTCTACG (SEQ | TCA CTG GGC ACG/3IABkFQ/-3′ | |
| | | (SEQ ID NO: 60) | ID NO: 61) | (SEQ ID NO: 62) | |
| CDC | InfA_2 | CAAGACCAATYCT | GCATTTTGGATAAA | 5′-/5FAM/TGC AGT CCT CGC | 1 |
| | | GTCACCTYTGAC | GCGTCTACG (SEQ | TCA CTG GGC ACG/3IABkFQ/-3′ | |
| | | (SEQ ID NO: 63) | ID NO: 64) | (SEQ ID NO: 65) | |
| CDC | InfB | TCCTCAAYTCACTC | CGGTGCTCTTGACC | 5′-/5FAM/CCA ATT CGA GCA | 1 |
| | | TTCGAGCG (SEQ | AAATTGG (SEQ ID | GCT GAA ACT GCG | |
| | | ID NO: 66) | NO: 67) | GTG/3IABkFQ/-3′ (SEQ ID NO: | |
| | | | | 68) | |

Human (Control)

| | | | | | |
|---|---|---|---|---|---|
| CDC | Rnase P | AGATTTGGACCTG | GAGCGGCTGTCTCC | 5′/CY5/TTC TGA CCT GAA | 1 |
| | | CGAGCG (SEQ ID | ACAAGT (SEQ ID | GGC TCT GCG CG/3IAbRQSp/-3′ | |
| | | NO: 69) | NO: 70) | (SEQ ID NO: 71) | |

SARS-CoV-2

| | | | | | |
|---|---|---|---|---|---|
| CDC | N1 | 5′-GAC CCC AAA | 5′-TCT GGT TAC | 5′-/FAM/-ACC CCG CAT TAC | 1 |
| | | ATC AGC GAA AT- | TGC CAG TTG AAT | GTT TGG TGG ACC-BHQ1-3′ | |
| | | 3′ (SEQ ID NO: 72) | CTG-3′ (SEQ ID NO: | (SEQ ID NO: 74) | |
| | | | 73) | | |
| CDC | N2 | 5′-TTA CAA ACA | 5′-GCG CGA CAT | 5′-/FAM/-ACA ATT TGC CCC | 1 |
| | | TTG GCC GCA AA- | TCC GAA GAA-3′ | CAG CGC TTC AG-BHQ1-3′ (SEQ | |
| | | 3′ (SEQ ID NO: 75) | (SEQ ID NO: 76) | ID NO: 77) | |
| WHO | Rdrp_ncov_ | GGTAACTGGTATG | CTGGTCAAGGTTAA | TCATACAAACCACGCCAGG | 2 |
| | ip4 | ATTTCG (SEQ ID | TATAGG (SEQ ID | [5′]Fam[3′]BHQ-1 (SEQ ID NO: | |
| | | NO: 78) | NO: 79) | 80) | |
| WHO | E_Sarbeco | ACAGGTACGTTAA | ATATTGCAGCAGTA | ACACTAGCCATCCTTACTGCGCT | 2 |
| | | TAGTTAATAGCGT | CGCACACA (SEQ ID | TCG[5′]Fam[3′]BHQ-1 (SEQ ID | |
| | | (SEQ ID NO: 81) | NO: 82) | NO: 83) | |

Respiratory Syncytial Virus A (RSV)

| | | | | | |
|---|---|---|---|---|---|
| MCW | RSVA1B988 | 5′-ACAATCTAAAAC | 5′-GTGTATTTGCTG | 5'-/5FAM/ATGCATAACTATACTC | 3 |
| | | AACAACTCTATGC | GATGACAG (SEQ ID | CATAGTCCAGATGGAGCCTGAA/ | |
| | | (SEQ ID NO: 84) | NO: 85) | 3IABkFQ/-3′ (SEQ ID NO: 86) | |

| | | |
|---|---|---|
| *Ref | 1 | https://www.cdc.gov/coronavirus/2019-ncov/lab/multiplex-primer-probes.html |
| | 2 | https://www.eurosurveillance.org/content/10.2807/1560- |
| | | 7917.ES2020.25.3.2000045;jsessionid=APVF7tuYDXOKu E4r9MabVsc.i- |
| | | 0b3d9850f4681504f-ecdclive |
| | 3 | U.S. Pat. No. 6,015,664 (Medical College of Wisconsin Research Foundation) |

In Table 2, the references N1 and N2 for sequences relevant to the SARS-CoV-2 genome indicate two target regions with the N gene of the virus genome, known as N1 and N2. The reference Rdrp_ncov_ip4 for sequences relevant to SARS-CoV-2 genome indicates a target region within the RdRp gene of the virus genome. The reference E Sarbeco for sequences relevant to SARS-CoV-2 genome indicates a target region within the E gene of the virus genome.

In various embodiments, primer sequences may be used to amplify genetic material present in a bio-sample prior to presenting the bio-sample to a sensor array chip. In various embodiments, amplification provides sufficient amounts of a target oligonucleotide and/or ensures that a particular target oligonucleotide is not present.

In various embodiments, primers amplify the target sequence that is complementary to the probe sequence. For example, forward and reverse primers may produce an amplification product from which a probe sequence can be generated. In specific embodiments, multiple pairs of primers are used to amplify a segment of the SARS-CoV-2 genome, such as a segment within the Rdrp region.

In various embodiments, a multiplexed sensor array comprises a plurality of sensor pixels, wherein the plurality of sensor pixels further comprise at least two, at least three, at least four, at least five, or more subsets of sensor pixels. The totality of the subsets of pixels necessarily equals the plurality. As an example, the chip architecture in FIG. 7 (left side of figure) is seen to comprise 256 sensor pixels that make up the plurality of pixels, arranged in a 16×16 square array on a semiconductor chip, and this plurality may comprise four distinguishable subsets of sensor pixels. The top left quadrant of 64 pixels may comprise the first subset of sensor pixels, the top right quadrant of 64 sensor pixels may comprise the second subset of sensor pixels, the bottom left quadrant of 64 sensor pixels may comprise the third subset of sensor pixels, and lastly, the bottom right quadrant of 64 sensor pixels may comprise the fourth subset of sensor pixels. Thus, in this hypothetical example, the plurality of 256 sensor pixels in the chip architecture of FIG. 7 comprises a first subset of 64 sensor pixels, a second subset of 64 sensor pixels, a third subset of 64 sensor pixels and a fourth subset of 64 sensor pixels. In this example, the four subsets are grouped and physically distinguishable simply by location of each subset, but a sensor array need not be limited as such, as subsets of sensor pixels may be interspersed, even randomly. However, the identity of the individual sensor pixels in the array will be decoded to yield a sensor array map and therefore the location of each of the sensors that make up a particular subset of sensor pixels will be known. In a regionally organized example, such as if the chip in FIG. 7 comprises four regionally distinct subsets of sensor pixels, the molecular electronics sensors in each subset can be exposed to a solution with a particular hybridization probe that conjugates to the sensors in that subset, with each subset exposed separately. In this hypothetical example, the first subset of sensor pixels may comprise molecular electronics sensors configured to detect a first pathogen, the second subset of sensor pixels may comprise molecular electronics sensors configured to detect a second pathogen, the third subset of sensor pixels may comprise molecular electronics sensors configured to detect a third pathogen, and the forth subset of sensor pixels may comprise molecular electronics sensors configured to detect a human gene and to thus confirm the bio-sample provided to the chip was indeed of human origin. In various embodiments, a sufficient number of subsets of sensor pixels are provided such that the identity of a pathogen can be established for a patient exhibiting respiratory symptoms that could be caused by any number of respiratory diseases (COVID-19, Influence A, Influenza B. RSV, etc.). At least one additional subset of sensor pixels is dedicated for a control function, such as to ensure the bio-sample is of human origin.

Table 3 sets forth additional probes that may be suitably functionalized for conjugation to a bridge molecule in a molecular electronics sensor. As discussed, conjugation may be at the 5′-end, at the 3′-end, or at a site between the 5′ and 3′ ends. The end or ends of the probe not conjugated to the bridge molecule may be functionalized with dye and/or quencher moieties as desired. In various embodiments, an azide moiety is provided on an end of a hybridization probe oligonucleotide for use in click-chemistry conjugation.

TABLE 3
Probes

| Reference | Probe | Identifier |
|---|---|---|
| N1Br31 | /5AzideN//iCy3/gcaccccgcattacgtttggtggaccctcag | SEQ ID NO: 87 |
| N1Br24 | /5AzideN//iCy3/accccgcattacgtttggtggacc | SEQ ID NO: 88 |
| N1Br21 | /5AzideN//iCy3/ccgcattacgtttggtggacc | SEQ ID NO: 89 |
| N1Br17 | /5AzideN//iCy3/gcattacgtttggtgga | SEQ ID NO: 90 |
| N1Br14 | /5AzideN//iCy3/gcattacgtttggt | SEQ ID NO: 91 |

In various embodiments, a multiplexed sensor for SNP genotyping comprises a plurality of sensor pixels, the plurality of sensor pixels further comprising at least two subsets of sensor pixels wherein the first subset of sensor pixels have sensors configured to detect a particular target sequence and the second subset of sensor pixels have sensors configured to detect a variant of the particular target sequence. Sensor arrays configured as such can be used to distinguish between variants of a pathogen, such as distinguishing between variants of the SARS-CoV-2 virus.

In various embodiments, a multiplexed sensor array comprises a plurality of sensor pixels, the plurality of sensor pixels further comprising at least two subsets of sensor pixels, including at least a first subset of sensor pixels and a second subset of sensor pixels. Each sensor pixel comprises a molecular electronics sensor comprising a hybridization probe bonded thereto. In various embodiments, the first subset of sensor pixels comprises sensors having a hybridization probe with at least 80%, at least 85%, at least 90%, at least 95%, or at least 98% sequence identity to SEQ ID NO: 62. In various embodiments, the second subset of sensor pixels comprises sensors having a hybridization probe with at least 80%, at least 85%, at least 90%, at least 95%, or at least 98% sequence identity to SEQ ID NO: 71. In this configuration, the sensor system can detect Influenza A1 and confirm that the bio-sample is of human origin. In various embodiments, the multiplexed sensor array further comprises a third subset of sensor pixels, each sensor pixel comprising a molecular electronics sensor comprising a hybridization probe bonded thereto, wherein the hybridization probe has at least 80%, at least 85%, at least 90%, at least 95%, or at least 98% sequence identity to any one of SEQ ID NOs: 65, 68, 74, 77, 80, 83 or 86. With the third subset of pixels, or even additional subsets of pixels (fourth, fifth, etc.), the sensor array is capable of distinguishing between pathogens and ensuring that the bio-sample is of human origin.

In various embodiments of the multiplexed sensor array above, a first subset of sensor pixels comprises sensors having hybridization probes with at least 80%, at least 85%, at least 90%, at least 95%, or at least 98% sequence identity to SEQ ID NO: 65. In various embodiments, a second subset of sensor pixels comprises sensors having hybridization probes with at least 80%, at least 85%, at least 90%, at least 95%, or at least 98% sequence identity to SEQ ID NO: 71. In this configuration, the sensor system can detect Influenza A2 and confirm that the bio-sample is of human origin. In various embodiments, the multiplexed sensor array further comprises a third subset of sensor pixels, each sensor pixel comprising a molecular electronics sensor comprising a hybridization probe bonded thereto, wherein the hybridization probe has at least 80%, at least 85%, at least 90%, at least 95%, or at least 98% sequence identity to any one of SEQ ID NOs: 62, 68, 74, 77, 80, 83 or 86. With the third subset of pixels, or even additional subsets of pixels (fourth, fifth, etc.), the sensor array is capable of distinguishing between pathogens and ensuring that the bio-sample is of human origin.

In various embodiments of the multiplexed sensor array above, a first subset of sensor pixels comprises sensors having hybridization probes with at least 80%, at least 85%, at least 90%, at least 95%, or at least 98% sequence identity to SEQ ID NO: 68. In various embodiments, a second subset of sensor pixels comprises sensors having hybridization probes with at least 80%, at least 85%, at least 90%, at least 95%, or at least 98% sequence identity to SEQ ID NO: 71. In this configuration, the sensor system can detect Influenza B and confirm that the bio-sample is of human origin. In various embodiments, the multiplexed sensor array further comprises a third subset of sensor pixels, each sensor pixel comprising a molecular electronics sensor comprising a hybridization probe bonded thereto, wherein the hybridization probe has at least 80%, at least 85%, at least 90%, at least 95%, or at least 98% sequence identity to any one of SEQ ID NOs: 62, 65, 74, 77, 80, 83 or 86. With the third subset of pixels, or even additional subsets of pixels (fourth, fifth, etc.), the sensor array is capable of distinguishing between pathogens and ensuring that the bio-sample is of human origin.

In various embodiments of the multiplexed sensor array above, a first subset of sensor pixels comprises sensors having hybridization probes with at least 80%, at least 85%, at least 90%, at least 95%, or at least 98% sequence identity to SEQ ID NO: 74. In various embodiments, a second subset of sensor pixels comprises sensors having hybridization probes with at least 80%, at least 85%, at least 90%, at least 95%, or at least 98% sequence identity to SEQ ID NO: 71. In this configuration, the sensor system can detect SARS-CoV-2 and confirm that the bio-sample is of human origin. In various embodiments, the multiplexed sensor array further comprises a third subset of sensor pixels, each sensor pixel comprising a molecular electronics sensor comprising a hybridization probe bonded thereto, wherein the hybridization probe has at least 80%, at least 85%, at least 90%, at least 95%, or at least 98% sequence identity to any one of SEQ ID NOs: 62, 65, 68, 77, 80, 83 or 86. With the third subset of pixels, or even additional subsets of pixels (fourth, fifth, etc.), the sensor array is capable of distinguishing between pathogens and ensuring that the bio-sample is of human origin.

In various embodiments of the multiplexed sensor array above, a first subset of sensor pixels comprises sensors having hybridization probes with at least 80%, at least 85%, at least 90%, at least 95%, or at least 98% sequence identity to SEQ ID NO: 77. In various embodiments, a second subset of sensor pixels comprises sensors having hybridization probes with at least 80%, at least 85%, at least 90%, at least 95%, or at least 98% sequence identity to SEQ ID NO: 71. In this configuration, the sensor system can detect SARS-CoV-2 and confirm that the bio-sample is of human origin. In various embodiments, the multiplexed sensor array further comprises a third subset of sensor pixels, each sensor pixel comprising a molecular electronics sensor comprising a hybridization probe bonded thereto, wherein the hybridization probe has at least 80%, at least 85%, at least 90%, at least 95%, or at least 98% sequence identity to any one of SEQ ID NOs: 62, 65, 68, 74, 80, 83 or 86. With the third subset of pixels, or even additional subsets of pixels (fourth, fifth, etc.), the sensor array is capable of distinguishing between pathogens and ensuring that the bio-sample is of human origin.

In various embodiments of the multiplexed sensor array above, a first subset of sensor pixels comprises sensors having hybridization probes with at least 80%, at least 85%, at least 90%, at least 95%, or at least 98% sequence identity to SEQ ID NO: 80. In various embodiments, a second subset of sensor pixels comprises sensors having hybridization probes with at least 80%, at least 85%, at least 90%, at least 95%, or at least 98% sequence identity to SEQ ID NO: 71. In this configuration, the sensor system can detect SARS-CoV-2 and confirm that the bio-sample is of human origin. In various embodiments, the multiplexed sensor array further comprises a third subset of sensor pixels, each sensor pixel comprising a molecular electronics sensor comprising a hybridization probe bonded thereto, wherein the hybridization probe has at least 80%, at least 85%, at least 90%, at least 95%, or at least 98% sequence identity to any one of SEQ ID NOs: 62, 65, 68, 74, 77, 83 or 86. With the third subset of pixels, or even additional subsets of pixels (fourth, fifth, etc.), the sensor array is capable of distinguishing between pathogens and ensuring that the bio-sample is of human origin.

In various embodiments of the multiplexed sensor array above, a first subset of sensor pixels comprises sensors having hybridization probes with at least 80%, at least 85%, at least 90%, at least 95%, or at least 98% sequence identity to SEQ ID NO: 83. In various embodiments, a second subset of sensor pixels comprises sensors having hybridization probes with at least 80%, at least 85%, at least 90%, at least 95%, or at least 98% sequence identity to SEQ ID NO: 71. In this configuration, the sensor system can detect SARS-CoV-2 and confirm that the bio-sample is of human origin. In various embodiments, the multiplexed sensor array further comprises a third subset of sensor pixels, each sensor pixel comprising a molecular electronics sensor comprising a hybridization probe bonded thereto, wherein the hybridization probe has at least 80%, at least 85%, at least 90%, at least 95%, or at least 98% sequence identity to any one of SEQ ID NOs: 62, 65, 68, 74, 77, 80, or 86. With the third subset of pixels, or even additional subsets of pixels (fourth, fifth, etc.), the sensor array is capable of distinguishing between pathogens and ensuring that the bio-sample is of human origin.

In various embodiments of the multiplexed sensor array above, a first subset of sensor pixels comprises sensors having hybridization probes with at least 80%, at least 85%, at least 90%, at least 95%, or at least 98% sequence identity to SEQ ID NO: 86. In various embodiments, a second subset of sensor pixels comprises sensors having hybridization probes with at least 80%, at least 85%, at least 90%, at least 95%, or at least 98% sequence identity to SEQ ID NO: 71. In this configuration, the sensor system can detect RSV and confirm that the bio-sample is of human origin. In various embodiments, the multiplexed sensor array further comprises a third subset of sensor pixels, each sensor pixel comprising a molecular electronics sensor comprising a hybridization probe bonded thereto, wherein the hybridization probe has at least 80%, at least 8500, at least 90%, at least 95%, or at least 98% sequence identity to any one of SEQ ID NOs: 62, 65, 68, 74, 77, 80, or 83. With the third subset of pixels, or even additional subsets of pixels (fourth, fifth, etc.), the sensor array is capable of distinguishing between pathogens and ensuring that the bio-sample is of human origin.

Primer Selection Considerations

TABLE 4 sets forth various primers.

TABLE 4
Primers

| Primer | Sequence Identifier |
|---|---|
| AAAGACAAGACCAATCTTGTCACCTCTGACTAAGG | SEQ ID NO: 92 |
| AAAGAGTTGATTTTTGTGGAAAG | SEQ ID NO: 93 |
| AAAGATGGCCATCGGATCCTCAACTCACTCTTCG | SEQ ID NO: 94 |
| AACCATTACAGATGCTGTAGACTGTGCACTTGACC | SEQ ID NO: 95 |
| AATTGGGAGCTTCGCAGC | SEQ ID NO: 96 |
| ACAACAGGATGGGAACAGTG | SEQ ID NO: 97 |
| ACACCGTTTCTATAGATTAGCTAATGAGTGTGCTC | SEQ ID NO: 98 |
| ACACTAGCCATCCTTACTGCGCTTCG | SEQ ID NO: 99 |
| ACAGGTACGTTAATAGTTAATAGCG | SEQ ID NO: 100 |
| ACAGGTACGTTAATAGTTAATAGCGT | SEQ ID NO: 101 |
| ACGTTGAAATCCTTCACTGT | SEQ ID NO: 102 |
| ACTCCTGGTGATTCTTCTTCAGGTTGGACAGCTGG | SEQ ID NO: 103 |
| ACTTGTGGAGACAGCCGCTC | SEQ ID NO: 104 |
| ACTTGTGGAGACAGCCGCTCACC | SEQ ID NO: 105 |
| ACTTGTTGCTACTGATGATCT | SEQ ID NO: 106 |
| ACTTGTTGCTACTGATGATCTTACAGTGGAGGATG | SEQ ID NO: 107 |
| AGAAAGTGAACTCGTAATCGGAGCTGTGATCCTTC | SEQ ID NO: 108 |
| AGAACTTCACAACTGCTCCTGCCA | SEQ ID NO: 109 |
| AGAAGTTATTTGACTCCTGGTGATT | SEQ ID NO: 110 |
| AGAATGGAGAACGCAGTGG | SEQ ID NO: 111 |
| AGAGGGAGACAATTAGACTGGC | SEQ ID NO: 112 |
| AGATTTGGACCTGCGAGCG | SEQ ID NO: 113 |
| AGCAGAAATGGAACAAGTTG | SEQ ID NO: 114 |
| AGGTGTCACTAAGCTATTCAACTG | SEQ ID NO: 115 |
| AGGTGTCACTAAGCTATTCAACTGG | SEQ ID NO: 116 |
| AGTGCTATTGGCAAAATTCAAGACTCACTTTCTTC | SEQ ID NO: 117 |
| AGTGGAGGATGAAAAAGATGG | SEQ ID NO: 118 |
| ATAACTACGATACGGGAGGGC | SEQ ID NO: 119 |
| ATCTGAATTCTTCTAGAGTTCCT | SEQ ID NO: 120 |
| ATCTTTGACTCAATTTCCTCACT | SEQ ID NO: 121 |
| ATGAACCACAAATCATTACTACA | SEQ ID NO: 122 |
| ATGGCGGTGTTTGCAGATTTGGACCTGCGAGCG | SEQ ID NO: 123 |
| ATGTACTCATTCGTTTCGGAAGAGACAGGTACGTT | SEQ ID NO: 124 |
| ATGTCTGATAATGGACCCCAAAATCAGCGAAATGC | SEQ ID NO: 125 |
| ATTGGCAAAATTCAAGACTCACTTTCTTCCACAGC | SEQ ID NO: 126 |
| ATTGGGAGCTTCGCAGCGT | SEQ ID NO: 127 |
| ATTGTGTGCGTACTGCTGCAATATTGTTAACGTGA | SEQ ID NO: 128 |
| CAAATTGGGAGCTTCGCAGCGTGTAGCAGGTGAC | SEQ ID NO: 129 |
| CAACGTGTTGTAGCTTGTC | SEQ ID NO: 130 |
| CAACGTGTTGTAGCTTGTCACACCGTTTCTATAGA | SEQ ID NO: 131 |
| CAAGACCAATCTTGTCACCTCTGAC | SEQ ID NO: 132 |
| CAAGCTGTCACGGCCAATGTTAATGCACTTTTATC | SEQ ID NO: 133 |
| CAAGGACCTGCCTAAAGAAATCAC | SEQ ID NO: 134 |
| CAATTTGGTCAAGAGCACCGATTATCACCAGAAGA | SEQ ID NO: 135 |
| CACAAAGGAATTTTTATGAACCACA | SEQ ID NO: 136 |
| CACAAAGGAATTTTTATGAACCACAAATCATTACT | SEQ ID NO: 137 |
| CACCTATCTCAGCGATCTGTCTA | SEQ ID NO: 138 |
| CACTGGTTTGTAACACAAAGGAA | SEQ ID NO: 139 |
| CAGATTCAACTGGCAGTAACC | SEQ ID NO: 140 |
| CAGATTCAACTGGCAGTAACCAGA | SEQ ID NO: 141 |
| CAGATTCAACTGGCAGTAACCAGAATGGAG | SEQ ID NO: 142 |
| CAGATTCAACTGGCAGTAACCAGAATGGAGAACGC | SEQ ID NO: 143 |
| CAGGTGGAACCTCATCAGGAGATGC | SEQ ID NO: 144 |
| CATCCATAGTTGCCTGACTCC | SEQ ID NO: 145 |
| CATGGAATGGCTAAAGACAAGACCAATCTTGTCAC | SEQ ID NO: 146 |
| CATTACAGATGCTGTAGACTGTGCACTTGACCCTC | SEQ ID NO: 147 |
| CATTATGTTAGGACACGCTAGTGTACAAGCAGAAA | SEQ ID NO: 148 |
| CATTTGTCAAGCTGTCACGGCCAATGTTAATGCA | SEQ ID NO: 149 |
| CCAAGGTTTACCCAATAATACT | SEQ ID NO: 150 |
| CCAATGTTAATGCACTTTTATCTACTGATGGTAAC | SEQ ID NO: 151 |
| CCAATTCGAGCAGCTGAAACTGCGGTG | SEQ ID NO: 152 |
| CCAATTTGGTCAAGAGCACCG | SEQ ID NO: 153 |
| CCACAACTGCTTATGCTAATAGTGT | SEQ ID NO: 154 |
| CCACGGAAGAACTTTATCTCT | SEQ ID NO: 155 |
| CCATATATTGAACAACCCAAAAGCATCA | SEQ ID NO: 156 |
| CCATCGGATCCTCAACTCA | SEQ ID NO: 157 |
| CCATCGGATCCTCAACTCAC | SEQ ID NO: 158 |
| CCGAACAACATGGATAGAGCAGTTAAACTATACAA | SEQ ID NO: 159 |
| CCGCATTACGTTTGGTGGACC | SEQ ID NO: 160 |
| CCTCTCTCAGAAACAAAGTGT | SEQ ID NO: 161 |
| CCTTCTTTTTACGTTTACTCTCG | SEQ ID NO: 162 |
| CGAGGACTGCAGCGTAGA | SEQ ID NO: 163 |
| CGCGATCAAAACAACGTCG | SEQ ID NO: 164 |
| CGTAGACGCTTTGTCCAAAATGC | SEQ ID NO: 165 |
| CGTGCCCAGTGAGCGAGGACTGCA | SEQ ID NO: 166 |
| CGTTCATCCATAGTTGCCTGACTCCC | SEQ ID NO: 167 |
| CTAAACGAACAAACTAAAATGTCTG | SEQ ID NO: 168 |
| CTAAATGGGAATGGGGACCCGAACAACATGGATAG | SEQ ID NO: 169 |
| CTAGGCATAATGGGAGAATACAGAGGTACACCAAG | SEQ ID NO: 170 |
| CTAGGTTTCAAACTTTACTTGC | SEQ ID NO: 171 |
| CTAGTGTACAAGCAGAAATGGAACAAGTTGTGGAG | SEQ ID NO: 172 |
| CTCACTTCTCTAGTGTAGTATTGGGCAATGCTGCT | SEQ ID NO: 173 |
| CTCAGATTCAACTGGCAGTAACCAGAATGG | SEQ ID NO: 174 |
| CTCAGATTCAACTGGCAGTAACCAGAATGGAGAAC | SEQ ID NO: 175 |
| CTCTAGTGTAGTATTGGGCAATGCT | SEQ ID NO: 176 |
| CTGTCACGGCCAATGTTA | SEQ ID NO: 177 |
| CTTCTCAACGTGCCACTCC | SEQ ID NO: 178 |
| GAAGAGGGAGACAATTAGACTGGCCACGGAAGAA | SEQ ID NO: 179 |
| GACCCCAAAATCAGCGAAAT | SEQ ID NO: 180 |
| GACGCTGTGACATCAAGGACCTGCCTAAAGAAATC | SEQ ID NO: 181 |
| GACTCAATTTCCTCACTTCTCTAGTGTAGTATTGG | SEQ ID NO: 182 |
| GACTTGTGGAGACAGCCGCTCACC | SEQ ID NO: 183 |
| GAGCTTCGCAGCGTGTAGCAGGTGACTCAGGTTTT | SEQ ID NO: 184 |
| GATTATCACCAGAAGAGGGAGACAATTAGACTGGC | SEQ ID NO: 185 |
| GCACTTGCCAGTTGCATG | SEQ ID NO: 186 |
| GCGGTGTTTGCAGATTTGGACCTGCGAGCG | SEQ ID NO: 187 |
| GCTGCAATATTGTTAACGTGAGTC | SEQ ID NO: 188 |
| GCTGCAATATTGTTAACGTGAGTCT | SEQ ID NO: 189 |
| GCTGGCCTAGGCATAATGG | SEQ ID NO: 190 |
| GCTGGTGCTGCAGCTTATTA | SEQ ID NO: 191 |
| GCTTTGTCCAAAATGCCCT | SEQ ID NO: 192 |
| GGAACCATTACAGATGCTGTAGACTGTGCACTTGA | SEQ ID NO: 193 |
| GGAAGAGACAGGTACGTTAATAGTTAATAGCGTAC | SEQ ID NO: 194 |
| GGACCCCAAAATCAGCGA | SEQ ID NO: 195 |
| GGACCCGAACAACATGGATAGAGCAGTTAAACTAT | SEQ ID NO: 196 |
| GGAGAATACAGAGGTACACC | SEQ ID NO: 197 |
| GGCACTATTCTGACCAGACCG | SEQ ID NO: 198 |
| GGCAGTAACCAGAATGGAG | SEQ ID NO: 199 |
| GGCGGTGTTTGCAGATTTGGACCTGCGAGCGGGTT | SEQ ID NO: 200 |
| GTACTGCTGCAATATTGTTAACGTGAGTCTTGTAA | SEQ ID NO: 201 |
| GTGAAATGGTCATGTGTGGCGG | SEQ ID NO: 202 |
| GTGACATCAAGGACCTGCCTA | SEQ ID NO: 203 |
| GTGACATCAAGGACCTGCCTAA | SEQ ID NO: 204 |
| GTGGAGACAGCCGCTCACC | SEQ ID NO: 205 |
| GTGGGGCGCGATCAAAACA | SEQ ID NO: 206 |
| GTGTGCGTACTGCTGCAATATTGTTAACGTGAGTC | SEQ ID NO: 207 |
| GTGTTCACGCTCACCGTG | SEQ ID NO: 208 |
| GTTAGGACACGCTAGTGTACAAGCAGAAATGGAAC | SEQ ID NO: 209 |
| GTTCACCGCTCTCACTCA | SEQ ID NO: 210 |
| GTTGCTAAACTTGTTGCTACTGATGATCTTACAGTG | SEQ ID NO: 211 |
| GTTTCGGAAGAGACAGGTACGTTAATAGTTAATAG | SEQ ID NO: 212 |
| TAATGGACCCCAAAATCAGCGAAATGCACC | SEQ ID NO: 213 |
| TAGACTGTGCACTTGACCCT | SEQ ID NO: 214 |
| TAGTGTACAAGCAGAAATGG | SEQ ID NO: 215 |
| TATAGTGCTATTGGCAAAATTCAAGACTCACTTTC | SEQ ID NO: 216 |
| TATGCTAATAGTGTTTTTAACATTTG | SEQ ID NO: 217 |
| TCAGCACCTCATGGTGTAG | SEQ ID NO: 218 |
| TCATCTTATGTCCTTCCCTC | SEQ ID NO: 219 |
| TCATGACGTTCGTGTTGT | SEQ ID NO: 220 |
| TCATTCGTTTCGGAAGAGA | SEQ ID NO: 221 |
| TCCTCAACTCACTCTTCGAGCG | SEQ ID NO: 222 |
| TCCTCGTGAAGGTGTCTTTG | SEQ ID NO: 223 |
| TCCTCGTGAAGGTGTCTTTGTTTC | SEQ ID NO: 224 |
| TCCTGGTGATTCTTCTTCAGGTTGGACAGCTGGTG | SEQ ID NO: 225 |
| TCGTGGACATCTTCGTATTGCTGGACACCATCTAG | SEQ ID NO: 226 |
| TCTAGTGTAGTATTGGGCAATGCT | SEQ ID NO: 227 |
| TCTGATAATGGACCCCAAAATCAGCGAAAT | SEQ ID NO: 228 |
| TCTGATAATGGACCCCAAAATCAGCGAAATGCACC | SEQ ID NO: 229 |
| TGACTCCTGGTGATTCTTCTTCAGGTTGGACAGCT | SEQ ID NO: 230 |
| TGCACTTTTATCTACTGATGGT | SEQ ID NO: 231 |
| TGGCTAAAGACAAGACCAATCTTGTCACCTCTGAC | SEQ ID NO: 232 |
| TGTGCTCAAGTATTGAGTGAAATGGTCATGTGTGG | SEQ ID NO: 233 |
| TGTGGGTTATCTTCAACCTAGGACT | SEQ ID NO: 234 |
| TGTGTGCGTACTGCTGCAATAT | SEQ ID NO: 235 |
| TGTTGCTACATCACGAACGC | SEQ ID NO: 236 |
| TGTTGCTACATCACGAACGCTTTC | SEQ ID NO: 237 |
| TGTTTGCAGATTTGGACCTGCGAGCGGGTT | SEQ ID NO: 238 |
| TTAGGACACGCTAGTGTAC | SEQ ID NO: 239 |
| TTATGAACCACAAATCATTACTACACACAACACA | SEQ ID NO: 240 |
| TTATTACAAATTGGGAGCTTCGCAGCGTGTAGCAG | SEQ ID NO: 241 |
| TTCACCGCTCTCACTCAA | SEQ ID NO: 242 |
| TTCTATAGATTAGCTAATGAGTGTG | SEQ ID NO: 243 |
| TTGGCAAAATTCAAGACTCACTTT | SEQ ID NO: 244 |
| TTTATGAACCACAAATCATTACTACACACAACACA | SEQ ID NO: 245 |
| TTTGGTGGACCCTCAGATTCAACTGGCAGTAAC | SEQ ID NO: 246 |

In various embodiments, primer pairs are restricted so as to:

(1) provide for effective priming under specified assay conditions, (e.g., buffer chemistry, temperature, etc.);

(2) not be impacted by known sequence variants in the target genome;

(3) reduce potential for “mispriming” against off-target genomes that might be present in the sample; and

(4) provide for a suitable hybridization probe target in the resulting amplicon.

In various embodiments, further primer selection considerations include:

(1) A selection of primers to target specified Tₘ relative to a specified buffer composition and specified assay temperature. For example, in the case of thermo cycling PCR, this may be a Tₘ=55° C. in a standard PCR buffer (Tris-HCL, KCL and MgCl₂).

(2) Primers should not overlap with variants that are likely to be present in the target viral genomes, specifically a given list of variants that are known or likely or suspected to be present in the infected population under test. In various embodiments, this includes a given list of variants to the reference viral genome that are present in commonly observed clades or strains, such as clades or strains that have been observed in more than 0.1% sequenced viral genomes for the virus of interest. In various embodiments, either primer do not overlap such variants at all, or where there is overlap, the variant does not intersect the last 10 bases of the 3′ end of a primer, and does not reduce the primer Tₘ by more than about 5° C.;

(3) Primers should be free from secondary structure with themselves or with other primers in the same reaction. In various embodiments, the primers do not have self-complementary segments longer than 3 bases at 3′ ends, or longer than 4 bases in arbitrary segments;

(4) In various embodiments, relevant genomes for off-target priming assessment are the human genome and (i) bacterial genomes that may contaminate human samples, such bacteria that reside in the human oral or nasal cavity, (ii) viral genomes that may contaminate human samples, and (iii) human respiratory viruses that may comprise an active infection, including off-target portions of the targeted viral genome that is the subject of the assay, including SARS-CoV-2 and known variants, Influenza A and B and known variants, and RSV and known variants. For all such off-target genomes to be assessed, individual primers will not have near matches against such genomes, and primer pairs will not have near matches within 2 kb of each other, such that an amplicon of that size would be produced. In various embodiments, “no near matches” may mean that the Tₘ of any such primer match is at least 10° C. below the assay temperature in the assay buffer; and

(5) The resulting target amplicons should have the segment between primer 3′ ends being at least 15 bases, and less than 100 bases, and such a segment must provide for a complementary hybridization probe preferably in the length range of 15 bases to 50 bases, and having a Tₘ within 5° C. of the assay temperature in the assay buffer, and which is free from secondary structure and free from any off target near matches in the amplicons that may be produced by the assay, the primers used in the assay, or in any off target genomes that may be present. In preferred embodiments, this may mean that any such secondary structure or off target matches for the hybridization probe have a Tₘ that is at least 10° C. below the assay temperature, in the assay buffer.

Additional Considerations

In various embodiments, a circuit comprises: a first electrode; a second electrode spaced apart from the first electrode; a molecular bridge attached to each of the first and second electrodes; and at least one nucleic acid hybridization probe conjugated to the molecular bridge.

In various embodiments, the molecular bridge comprises a double-stranded oligonucleotide, a protein alpha-helix, a peptide, a carbon nanotube, a graphene nanoribbon, a bacterial nanowire, or a conducting polymer.

In various embodiments, the at least one nucleic acid hybridization probe is conjugated to the molecular bridge at the 5′ end of the at least one nucleic acid hybridization probe, the 3′ end of the at least one nucleic acid hybridization probe, or at a nucleic acid base internal to the at least one nucleic acid hybridization probe.

In various embodiments, the at least one nucleic acid hybridization probe comprises from about 8 to about 25 nucleic acid bases.

In various embodiments, the circuit comprises a plurality of nucleic acid hybridization probes.

In various embodiments, each of the plurality of nucleic acid hybridization probes has a unique nucleic acid sequence.

In various embodiments, the at least one nucleic acid hybridization probe comprises a single-stranded DNA.

In various embodiments, the circuit further comprises a gate electrode.

In various embodiments, the circuit resides on a CMOS chip.

In various embodiments, a molecular sensor a circuit further comprising a first electrode; a second electrode spaced apart from the first electrode; a molecular bridge attached to each of the first and second electrodes; and at least one nucleic acid hybridization probe conjugated to the molecular bridge, wherein the circuit resides on a CMOS chip, and wherein the molecular sensor is housed in a cartridge optimized for sampling at least one source of a target nucleic acid molecule.

In various embodiments, a method of detecting a foreign nucleic acid molecule in solution comprises: (1) providing a circuit comprising a first electrode; a second electrode spaced apart from the first electrode; a molecular bridge attached to each of the first and second electrodes; and at least one nucleic acid hybridization probe conjugated to the molecular bridge, wherein the at least one nucleic acid hybridization probe is capable of hybridizing to the foreign nucleic acid molecule; (2) initiating at least one of a voltage or a current through the circuit; (3) exposing the circuit to the solution for a defined period of time; and (4) measuring electrical signals through the circuit as the at least one nucleic hybridization probe hybridizes with the foreign nucleic acid molecule, wherein the electrical signals are processed to provide information on the underlying sequence of the foreign nucleic acid molecule.

In various embodiments, the solution to be exposed to the circuit is first prepared by PCR methods of amplification to amplify the foreign nucleic acid present in the solution.

In various embodiments, the method further comprises transferring the provided information to a data processor.

In various embodiments, the electrical signals comprise a distinguishable signal event that correlates with the at least one nucleic acid hybridization probe being a reverse complement of the foreign nucleic acid molecule.

In various embodiments, the foreign nucleic acid molecule is associated with a viral genome.

In various embodiments, the viral genome comprises a SARS-CoV-2 genome, or a mutant thereof.

In various embodiments, a method of detecting a viral infection in an individual comprises: obtaining a biological sample from the individual; providing a circuit comprising a first electrode; a second electrode spaced apart from the first electrode; a molecular bridge attached to each of the first and second electrodes; and at least one nucleic acid hybridization probe conjugated to the molecular bridge, wherein the at least one nucleic acid hybridization probe is capable of hybridizing to a viral nucleic acid molecule contained in the biological sample; initiating at least one of a voltage or a current through the circuit; exposing the circuit to the biological sample for a defined period of time; and measuring electrical signals through the circuit as the at least one nucleic hybridization probe hybridizes with the viral nucleic acid molecule contained in the biological sample, wherein the electrical signals are processed to provide information on the presence or absence of the viral nucleic acid molecule.

In various embodiments, the viral nucleic acid molecule is associated with a SARS-CoV-2 genome, or a mutant thereof.

In various embodiments, the method further comprises treating the individual with an effective amount of an anti-viral therapy or anti-viral regimen.

In various embodiments, a sensor device comprises: a first molecular electronics sensor configured in a first sensor pixel, the first sensor comprising a first electrode; a second electrode spaced-apart from the first electrode by a first nanogap; a first bridge molecule bridging the first nanogap and electrically connecting the first electrode and the second electrode; and a first hybridization probe comprising a first oligonucleotide sequence conjugated to the first bridge molecule; a second molecular electronics sensor configured in a second sensor pixel, the second sensor comprising a third electrode; a fourth electrode spaced-apart from the third electrode by a second nanogap; a second bridge molecule bridging the second nanogap and electrically connecting the third electrode and the fourth electrode; and a second hybridization probe comprising a second oligonucleotide sequence conjugated to the second bridge molecule.

In various embodiments, the sensor device further comprises a third molecular electronics sensor configured in a third sensor pixel, the third sensor comprising a fifth electrode; a sixth electrode spaced-apart from the fifth electrode by a third nanogap; a third bridge molecule bridging the third nanogap and electrically connecting the fifth electrode and the sixth electrode; and a third hybridization probe comprising a third oligonucleotide sequence conjugated to the third bridge molecule.

In various embodiments, the first oligonucleotide sequence comprises a DNA sequence complementary to a segment of a first pathogen genome.

In various embodiments, the second oligonucleotide sequence comprises a DNA sequence complementary to a segment of a second pathogen genome.

In various embodiments, the third oligonucleotide sequence comprises a DNA sequence complementary to a segment of a constitutively expressed human gene.

In various embodiments, at least one of the first bridge molecule, the second bridge molecule and the third bridge molecule comprise a peptide having at least 80%, at least 85%, at least 90%, at least 95%, or at least 98% sequence identity to SEQ ID NO: 3.

In various embodiments, the first, second, third, fourth, fifth and sixth electrodes comprise Ag, Al, Au, Cr, Cu, Ni, Ga, Ti, Pt, Pd, Rb, Rh, or Ru.

In various embodiments, the first sensor further comprises a first decoding probe comprising a fourth oligonucleotide sequence conjugated to either the first hybridization probe or to the first bridge molecule.

In various embodiments, the second sensor further comprises a second decoding probe comprising a fifth oligonucleotide sequence conjugated to either the second hybridization probe or to the second bridge molecule.

In various embodiments, the third sensor further comprises a third decoding probe comprising a sixth oligonucleotide sequence conjugated to either the third hybridization probe or to the third bridge molecule.

In various embodiments, a plurality of first sensor pixels and a plurality of second sensor pixels are configured as a sensor pixel array deployed on a CMOS chip.

In various embodiments, a plurality of first sensor pixels, a plurality of second sensor pixels, and a plurality of third sensor pixels are configured as a sensor pixel array deployed on a CMOS chip.

In various embodiments, the first pathogen is SARS-CoV-2.

In various embodiments, the second pathogen is an Influenza A or Influenza B virus.

In various embodiments, the human gene comprises β-actin.

In various embodiments, a method of detecting a target oligonucleotide sequence in a bio-sample comprises:

providing a sensor device comprising a plurality of sensor pixels configured in an array on a semiconductor chip, the plurality of sensor pixels comprising at least a first subset of sensor pixels; wherein each sensor pixel in the plurality of sensor pixels comprises a molecular electronics sensor further comprising: a first electrode; a second electrode spaced-apart from the first electrode by a nanogap; a bridge molecule having a first end and a second end, the first end coupled to the first electrode and the second end coupled to the second electrode; and a hybridization probe having an oligonucleotide sequence conjugated to the bridge molecule; wherein each molecular electronics sensor in the first subset of sensor pixels includes a first hybridization probe comprising a first oligonucleotide sequence capable of hybridizing to the target oligonucleotide sequence;

initiating at least one of a voltage or a current through each sensor pixel in the plurality of sensor pixels;

exposing the plurality of sensor pixels to the bio-sample; and

measuring electrical signals from the first subset of sensor pixels as the target oligonucleotide sequence and the first hybridization probe engage in hybridization, wherein the electrical signals provide a signature indicating the target oligonucleotide sequence is present in the bio-sample.

In various embodiments of the method, the bio-sample is first amplified by methods designed to amplify at least the target oligonucleotide sequence if it is present in the bio-sample, prior to exposure of the plurality of sensor pixels to the bio-sample. In various embodiments, the PCR method utilizes at least one of the primers set forth herein.

In various embodiments of the method, the target oligonucleotide sequence is a segment of a genome of a first pathogen. In various embodiments, the first oligonucleotide sequence is complementary to a segment of a genome from a first pathogen. In various embodiments, the first pathogen is SARS-CoV-2, an Influenza A strain, an Influenza B strain, or RSV.

In various embodiments of the method, the measured electrical signals comprise perturbations in a voltage or current over time from the first set of sensor pixels in the plurality of sensor pixels, wherein the perturbations collectively provide a signature indicative the target oligonucleotide sequence is either present or not present in the bio-sample.

In various embodiments of the method, the method further comprises confirming that the bio-sample is of human origin, wherein the plurality of sensor pixels further comprises a second subset of sensor pixels and each molecular electronics sensor in the second subset of sensor pixels includes a second hybridization probe configured to detect a segment of an expressed human gene. In various embodiments, the second oligonucleotide sequence is complementary to a segment of a constitutively expressed human gene. In various embodiments, the human gene is human RNase P gene.

In various embodiments of the method, additional subsets of sensor pixels are provided in the sensor device so that the target oligonucleotide sequence can not only be detected but can also be attributed to a particular pathogen such as SARS-CoV-2, Influenza A, Influenza B or RSV. In various embodiments, electrical signals from each subset of sensor pixels are measured, and the electrical signals interpreted as to which subset of sensor pixels engaged in hybridization events with the target oligonucleotide sequence, thus identifying the pathogen source of the target hybridization sequence.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1-27. (canceled)

28. A sensor device comprising:
a plurality of sensor pixels configured in an array on a semiconductor chip, the plurality of sensor pixels comprising at least a first subset of sensor pixels and a second subset of sensor pixels;
wherein each sensor pixel in the plurality of sensor pixels comprises a molecular electronics sensor further comprising: a first electrode; a second electrode spaced-apart from the first electrode by a nanogap; a bridge molecule having a first end and a second end, the first end coupled to the first electrode and the second end coupled to the second electrode; and a hybridization probe having an oligonucleotide sequence conjugated to the bridge molecule;
wherein each molecular electronics sensor in the first subset of sensor pixels includes a first hybridization probe comprising a first oligonucleotide sequence complementary to a segment of a genome of a first pathogen;
wherein each molecular electronics sensor in the second subset of sensor pixels includes a second hybridization probe comprising a second oligonucleotide sequence complementary to a segment of a genome of a second pathogen or complementary to a segment of an expressed human gene;
wherein the first pathogen is SARS-CoV-2;
wherein the second pathogen is an Influenza A virus strain, an Influenza B virus strain, or Respiratory Syncytial Virus (RSV); and
wherein each bridge molecule in the plurality of sensor pixels comprises a polypeptide having at least 95% sequence identity to SEQ ID NO: 3.

29. The sensor device of claim 28, wherein each bridge molecule in the plurality of sensor pixels comprises a polypeptide having the sequence of SEQ ID NO: 3.

30. The sensor device of claim 28, wherein the expressed human gene is a constitutively expressed human gene.

31. The sensor device of claim 28, wherein the expressed human gene is human RNase P gene or β-actin gene.

32. The sensor device of claim 28, wherein the first oligonucleotide sequence has at least 95% sequence identity to any one of SEQ ID NOs: 74, 77, 80, 83, or 87-246.

33. The sensor device of claim 32, wherein the first oligonucleotide sequence comprises any one of SEQ ID NOs: 74, 77, 80, 83, or 87-246.

34. The sensor device of claim 28, wherein the second oligonucleotide sequence has at least 95% sequence identity to any one of SEQ ID NOs: 62, 65, 68, 71, or 86.

35. The sensor device of claim 34, wherein the second oligonucleotide sequence comprises any one of SEQ ID NOs: 62, 65, 68, 71, or 86.

36. The sensor device of claim 28, wherein each molecular electronics sensor in the first subset of sensor pixels further comprises a first decoding probe bonded to either the bridge molecule or the first hybridization probe in the molecular electronics sensor, and wherein each molecular electronics sensor in the second subset of sensor pixels further comprises a second decoding probe bonded to either the bridge molecule or the second hybridization probe in the molecular electronics sensor.

37. The sensor device of claim 28, wherein the plurality of sensor pixels further comprises a third subset of sensor pixels, and wherein each molecular electronics sensor in the third subset of sensor pixels includes a third hybridization probe comprising a third oligonucleotide sequence complementary to a segment of a genome of a third pathogen.

38. The sensor device of claim 37, wherein the first pathogen is SARS-CoV-2, the second oligonucleotide sequence is complementary to a segment of a genome of a constitutively expressed human gene, and the third pathogen is an Influenza A virus strain, an Influenza B virus strain, or Respiratory Syncytial Virus (RSV).

39. The sensor device of claim 37, wherein the first oligonucleotide sequence has at least 95% sequence identity to any one of SEQ ID NOs: 74, 77, 80, 83, or 87-246, wherein the second oligonucleotide sequence has at least 95% sequence identity to SEQ ID NO: 71, and wherein the third oligonucleotide sequence has at least 95% sequence identity to any one of SEQ ID NOs: 62, 65, 68, or 86.

40. The sensor device of claim 39, wherein the first oligonucleotide sequence comprises any one of SEQ ID NOs: 74, 77, 80, 83, or 87-246, wherein the second oligonucleotide sequence comprises SEQ ID NO: 71, and wherein the third oligonucleotide sequence comprises any one of SEQ ID NOs: 62, 65, 68, or 86.
